SEQUENCE LISTING (218 sequences)

SEQ ID NO: 1 - protein, 839 aa, Human [sequence omitted]

SEQ ID NO: 2 - protein, 16 aa, Artificial Sequence
  Amino acid sequence of the isolated peptide OTV16, not including additional modifications present for the purposes of antibody generation
YRPVDGLPPFKMEKTG

SEQ ID NO: 3 - protein, 18 aa, Artificial Sequence
  Amino acid sequence of the isolated peptide OTV16, including additional modifications present for the purposes of antibody generation
  MISC_FEATURE (1)..(18): The peptide is amidated at the C-terminus and has a Propargyl group (Pra-) at the N-terminus
  MISC_FEATURE (1)..(18): This peptide is cyclic, with the peptide being connected (cyclized) via the terminal cysteine residues
CYRPVDGLPPFKMEKTGC

SEQ ID NO: 4 - protein, 16 aa, Artificial Sequence
  Amino acid sequence of the isolated peptide NHP-OTV16, not including additional modifications
YRPVDGLPPFKMEKTE

SEQ ID NO: 5 - protein, 18 aa, Artificial Sequence
  Amino acid sequence of the isolated peptide NHP-OTV16, including additional modifications
  MISC_FEATURE (1)..(18): The peptide is amidated at the C-terminus and has a Propargyl group (Pra-) at the N-terminus
  MISC_FEATURE (1)..(18): This peptide is cyclic, with the peptide being connected (cyclized) via the terminal cysteine residues
CYRPVDGLPPFKMEKTEC

SEQ ID NO: 6 - protein, 16 aa, Artificial Sequence
  Amino acid sequence of the isolated peptide rOTV16, not including additional modifications
YRPVEGLPPYKLKNTG

SEQ ID NO: 7 - protein, 18 aa, Artificial Sequence
  Amino acid sequence of the isolated peptide rOTV16, including additional modifications
  MISC_FEATURE (1)..(18): The peptide is amidated at the C-terminus and has a Propargyl group (Pra-) at the N-terminus
  MISC_FEATURE (1)..(18): This peptide is cyclic, with the peptide being connected (cyclized) via the terminal cysteine residues
CYRPVEGLPPYKLKNTGC

SEQ ID NO: 8 - protein, 17 aa, Artificial Sequence
  Amino acid sequence of the isolated peptide mOTV16, not including additional modifications
YRPVEGLPPYKLNNTVG

SEQ ID NO: 9 - protein, 19 aa, Artificial Sequence
  Amino acid sequence of the isolated peptide mOTV16, including additional modifications
  MISC_FEATURE (1)..(19): The peptide is amidated at the C-terminus and has a Propargyl group (Pra-) at the N-terminus
  MISC_FEATURE (1)..(19): This peptide is cyclic, with the peptide being connected (cyclized) via the terminal cysteine residues
CYRPVEGLPPYKLNNTVGC

SEQ ID NO: 10 - protein, 839 aa, Macaca mulatta
MKKWSSTDSGAAADPLQQDACPDPPDGDPNSRPPPAKPQLSTAKSRTRLFGKGDSEEAFLVDCPHEEGELASCPTITVSPVITIQRPGDGPTGARLPSQDSVATSTERTLRLYDRRSIFEAVAQNNCQDLESLLLFLQKSEKHLTDNEFKDPETGKTCLLKAMLNLHDGQNDTIPLLLEIARQTGSLKEFVNASYTDSYYKGQTALHIAIERRNMGLVTLLVENGADVQAAANGDFFKKTKGRPGFYFGELPLSLAACTNQLGIVKFLLQNSWQPADISARDSVGNTVLHALVEVADNTADNTKFVTSMYNEILILGAKLHPTLKLEELTNKKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRTFYFNFLVYCLYMIIFTMAAYYRPVDGLPPFKMEKTEDYFRVTGEILSVLGGVYFFFRGIQYFLQRRPSMKTLFVDSYSEMLFFLQSLFMLATVVLYFSHLKEYVASMVFSLALGWTNMLYYTRGFQQMGIYAVMIEKMILRDLCRFMFVYVVFLFGFSTAVVTLIEDGKNNSLPSESTSHRWRGPGCRPPDSSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGYTPDGKDDYRWCFRVDEVNWTTWNTNVGIINEDPGNCEGVKRTLSFSLRSSRVSARHWKNFALVPLLREASARDRPSAQPEEVHLRQFSGSLRPEDAEVFKSPPASGEK

SEQ ID NO: 11 - protein, 839 aa, Mouse
MEKWASLDSDESEPPAQENSCPDPPDRDPNSKPPPAKPHIFATRSRTRLFGKGDSEEASPMDCPYEEGGLASCPIITVSSVVTLQRSVDGPTCLRQTSQDSVSTGVETPPRLYDRRSIFDAVAQSNCQELESLLSFLQKSKKRLTDSEFKDPETGKTCLLKAMLNLHNGQNDTIALLLDIARKTDSLKQFVNASYTDSYYKGQTALHIAIERRNMALVTLLVENGADVQAAANGDFFKKTKGRPGFYFGELPLSLAACTNQLAIVKFLLQNSWQPADISARDSVGNTVLHALVEVADNTADNTKFVTNMYNEILILGAKLHPTLKLEELTNKKGLTPLALAASSGKIGVLAYILQREIHEPECRHLSRKFTEWAYGPVHSSLYDLSCIDTCEKNSVLEVIAYSSSETPNRHDMLLVEPLNRLLQDKWDRFVKRIFYFNFFVYCLYMIIFTTAAYYRPVEGLPPYKLNNTVGDYFRVTGEILSVSGGVYFFFRGIQYFLQRRPSLKSLFVDSYSEILFFVQSLFMLVSVVLYFSHRKEYVASMVFSLAMGWTNMLYYTRGFQQMGIYAVMIEKMILRDLCRFMFVYLVFLFGFSTAVVTLIEDGKNNSLPVESPPHKCRGSACRPGNSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGFTPDGKDDFRWCFRVDEVNWTTWNTNVGIINEDPGNCEGVKRTLSFSLRSGRVSGRNWKNFALVPLLRDASTRDRHSTQPEEVQLKHYTGSLKPEDAEVFKDSMAPGEK

SEQ ID NO: 12 - protein, 838 aa, Rat
MEQRASLDSEESESPPQENSCLDPPDRDPNCKPPPVKPHIFTTRSRTRLFGKGDSEEASPLDCPYEEGGLASCPIITVSSVLTIQRPGDGPASVRPSSQDSVSAGEKPPRLYDRRSIFDAVAQSNCQELESLLPFLQRSKKRLTDSEFKDPETGKTCLLKAMLNLHNGQNDTIALLLDVARKTDSLKQFVNASYTDSYYKGQTALHIAIERRNMTLVTLLVENGADVQAAANGDFFKKTKGRPGFYFGELPLSLAACTNQLAIVKFLLQNSWQPADISARDSVGNTVLHALVEVADNTVDNTKFVTSMYNEILILGAKLHPTLKLEEITNRKGLTPLALAASSGKIGVLAYILQREIHEPECRHLSRKFTEWAYGPVHSSLYDLSCIDTCEKNSVLEVIAYSSSETPNRHDMLLVEPLNRLLQDKWDRFVKRIFYFNFFVYCLYMIIFTAAAYYRPVEGLPPYKLKNTVGDYFRVTGEILSVSGGVYFFFRGIQYFLQRRPSLKSLFVDSYSEILFFVQSLFMLVSVVLYFSQRKEYVASMVFSLAMGWTNMLYYTRGFQQMGIYAVMIEKMILRDLCRFMFVYLVFLFGFSTAVVTLIEDGKNNSLPMESTPHKCRGSACKPGNSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGFTPDGKDDYRWCFRVDEVNWTTWNTNVGIINEDPGNCEGVKRTLSFSLRSGRVSGRNWKNFALVPLLRDASTRDRHATQQEEVQLKHYTGSLKPEDAEVFKDSMVPGEK

SEQ ID NO: 13 - DNA, 327 bp, Rabbit
cagtcggtggaggagtccgggggtcgcccggtcacgcctgggacacccctgacactcacctgcacagtctctggtatcgacctcagtaggtatgccgtggactgggtccgccaggctccagggaaggggctggaatggatcggaatcattcttactagtggtaagatatcatacgcgagctgggtgaaaggccgattcaccatctccaaaacctcgaccacggtggatctgaaaatgaccagtctgacaaccgaggacacggccacttatttctgtgccagacattttgatttgtggggccaaggcaccctggtcaccgtctcctca

SEQ ID NO: 14 - DNA, 327 bp, Rabbit
caagtgctgacccagactgcatcgcccgtgtctgcggctgtgggaggcacagtcaccatcaactgccagtccagtcagagtgtttataataacaacaacttagcctggtatcagcagaaaccagggcagcctcccaagctcctgatctatgattcatccaaactggaatcaggggtcccatcgcggtttaaaggcagtggatctgggacacagttcactctcatcatcagcgacctggagtgtgacgatgctgccacttattactgtgcaggcggttatagtggtgttattgctagtttcggcggagggaccgaggtggtggtcaaa

SEQ ID NO: 15 - protein, 109 aa, Rabbit
QSVEESGGRPVTPGTPLTLTCTVSGIDLSRYAVDWVRQAPGKGLEWIGIILTSGKISYASWVKGRFTISKTSTTVDLKMTSLTTEDTATYFCARHFDLWGQGTLVTVSS

SEQ ID NO: 16 - protein, 109 aa, Rabbit
QVLTQTASPVSAAVGGTVTINCQSSQSVYNNNNLAWYQQKPGQPPKLLIYDSSKLESGVPSRFKGSGSGTQFTLIISDLECDDAATYYCAGGYSGVIASFGGGTEVVVK

SEQ ID NO: 17 - protein, 5 aa, Rabbit
RYAVD

SEQ ID NO: 18 - protein, 16 aa, Rabbit
IILTSGKISYASWVKG

SEQ ID NO: 19 - protein, 4 aa, Rabbit
HFDL

SEQ ID NO: 20 - protein, 13 aa, Rabbit
QSSQSVYNNNNLA

SEQ ID NO: 21 - protein, 7 aa, Rabbit
DSSKLES

SEQ ID NO: 22 - protein, 10 aa, Rabbit
AGGYSGVIAS

SEQ ID NO: 23 - protein, 29 aa, Rabbit
QSVEESGGRPVTPGTPLTLTCTVSGIDLS

SEQ ID NO: 24 - protein, 14 aa, Rabbit
WVRQAPGKGLEWIG

SEQ ID NO: 25 - protein, 30 aa, Rabbit
RFTISKTSTTVDLKMTSLTTEDTATYFCAR

SEQ ID NO: 26 - protein, 11 aa, Rabbit
WGQGTLVTVSS

SEQ ID NO: 27 - protein, 22 aa, Rabbit
QVLTQTASPVSAAVGGTVTINC

SEQ ID NO: 28 - protein, 15 aa, Rabbit
WYQQKPGQPPKLLIY

SEQ ID NO: 29 - protein, 32 aa, Rabbit
GVPSRFKGSGSGTQFTLIISDLECDDAATYYC

SEQ ID NO: 30 - protein, 10 aa, Rabbit
FGGGTEVVVK

SEQ ID NO: 31 - DNA, 339 bp, Rabbit
cagtcggtggaggagtccgggggtcgcctggtaacgcctgggacaccactgacactcacctgcacagtctctggattctctcttagtacctatgcgatggcctgggtccgccaggctccggggaagggtctggagtggatcggaatcattggtcagagttctaaaacatactacgcgatttgggcgaaaggccgattcaccatctccagaacctcgaccacggtggatctgaagatccccagtccgacaaccgaggacacggccacctatttctgtgccagagggagtacttatgttttggacatgtggggcccgggcaccctggtcaccgtctcctta

SEQ ID NO: 32 - DNA, 327 bp, Rabbit
gccgtcgtgatgacccagactgcatcccccgtgtctgcagctgtgggaggcacagtcaccatcaagtgccaggccagtcagagcattagtagctacttagcctggtatcaacagaaaccagggcagcctcccaagctcctgatcgctgatgcatcgaagctggtatctggggtcccatcgcgtttcaaaggcagtggatctgggacagaattcactctcaccatcagcgacctggagtgtgccgatgctgccacttactactgtcagaactattatggttcttctgattttggtactttcggcggagggaccgaggtggtggtcgag

SEQ ID NO: 33 - protein, 113 aa, Rabbit
QSVEESGGRLVTPGTPLTLTCTVSGFSLSTYAMAWVRQAPGKGLEWIGIIGQSSKTYYAIWAKGRFTISRTSTTVDLKIPSPTTEDTATYFCARGSTYVLDMWGPGTLVTVSL

SEQ ID NO: 34 - protein, 109 aa, Rabbit
AVVMTQTASPVSAAVGGTVTIKCQASQSISSYLAWYQQKPGQPPKLLIADASKLVSGVPSRFKGSGSGTEFTLTISDLECADAATYYCQNYYGSSDFGTFGGGTEVVVE

SEQ ID NO: 35 - protein, 5 aa, Rabbit
TYAMA

SEQ ID NO: 36 - protein, 16 aa, Rabbit
IIGQSSKTYYAIWAKG

SEQ ID NO: 37 - protein, 8 aa, Rabbit
GSTYVLDM

SEQ ID NO: 38 - protein, 11 aa, Rabbit
QASQSISSYLA

SEQ ID NO: 39 - protein, 7 aa, Rabbit
DASKLVS

SEQ ID NO: 40 - protein, 11 aa, Rabbit
QNYYGSSDFGT

SEQ ID NO: 41 - protein, 29 aa, Rabbit
QSVEESGGRLVTPGTPLTLTCTVSGFSLS

SEQ ID NO: 42 - protein, 14 aa, Rabbit
WVRQAPGKGLEWIG

SEQ ID NO: 43 - protein, 30 aa, Rabbit
RFTISRTSTTVDLKIPSPTTEDTATYFCAR

SEQ ID NO: 44 - protein, 11 aa, Rabbit
WGPGTLVTVSL

SEQ ID NO: 45 - protein, 23 aa, Rabbit
AVVMTQTASPVSAAVGGTVTIKC

SEQ ID NO: 46 - protein, 15 aa, Rabbit
WYQQKPGQPPKLLIA

SEQ ID NO: 47 - protein, 32 aa, Rabbit
GVPSRFKGSGSGTEFTLTISDLECADAATYYC

SEQ ID NO: 48 - protein, 10 aa, Rabbit
FGGGTEVVVE

SEQ ID NO: 49 - DNA, 354 bp, Rabbit
cagtcgctggaggaggctgggggtcgcctggtaacgcctggaggatccctgacactcacctgcacagcctctggattgtccctcagcagctacgacatgaactgggtccgccaggctccagggaaggggctggaattcatcggattcattaatactcgtggtaccgcatactacgcgagctgggcaaaaggccgattcaccatctccagaacctcgaccacggtggatctgacaatgaccagtccgacaaccgaggacacggccacctatttctgtgccagagatattcctgattggaatattggtcatgtttttgatctctggggcccaggcaccctggtcaccgtctcctca

SEQ ID NO: 50 - DNA, 330 bp, Rabbit
gcctatgatatgacccagactccagcctctgtggaggtaactatgggaggcgcagtcaccatcaattgccaggccagtcagagcattaccaatgaattagcctggtatcagcagaaaccagggcagcgtcccaggctcctgatttaccaggcttctgctctggcatctggggtcccatcgcggttcaaaggcagtggatatgggacacagttcactctcaccatcagcgacctggagtgtgccgatgctgccacttactactgtcaacagggttatactagtaataatattgtcaatgctttcggcggagggaccgaggtggtggtcaaa

SEQ ID NO: 51 - protein, 118 aa, Rabbit
QSLEEAGGRLVTPGGSLTLTCTASGLSLSSYDMNWVRQAPGKGLEFIGFINTRGTAYYASWAKGRFTISRTSTTVDLTMTSPTTEDTATYFCARDIPDWNIGHVFDLWGPGTLVTVSS

SEQ ID NO: 52 - protein, 110 aa, Rabbit
AYDMTQTPASVEVTMGGAVTINCQASQSITNELAWYQQKPGQRPRLLIYQASALASGVPSRFKGSGYGTQFTLTISDLECADAATYYCQQGYTSNNIVNAFGGGTEVVVK

SEQ ID NO: 53 - protein, 5 aa, Rabbit
SYDMN

SEQ ID NO: 54 - protein, 16 aa, Rabbit
FINTRGTAYYASWAKG

SEQ ID NO: 55 - protein, 13 aa, Rabbit
DIPDWNIGHVFDL

SEQ ID NO: 56 - protein, 11 aa, Rabbit
QASQSITNELA

SEQ ID NO: 57 - protein, 7 aa, Rabbit
QASALAS

SEQ ID NO: 58 - protein, 12 aa, Rabbit
QQGYTSNNIVNA

SEQ ID NO: 59 - protein, 29 aa, Rabbit
QSLEEAGGRLVTPGGSLTLTCTASGLSLS

SEQ ID NO: 60 - protein, 14 aa, Rabbit
WVRQAPGKGLEFIG

SEQ ID NO: 61 - protein, 30 aa, Rabbit
RFTISRTSTTVDLTMTSPTTEDTATYFCAR

SEQ ID NO: 62 - protein, 11 aa, Rabbit
WGPGTLVTVSS

SEQ ID NO: 63 - protein, 23 aa, Rabbit
AYDMTQTPASVEVTMGGAVTINC

SEQ ID NO: 64 - protein, 15 aa, Rabbit
WYQQKPGQRPRLLIY

SEQ ID NO: 65 - protein, 32 aa, Rabbit
GVPSRFKGSGYGTQFTLTISDLECADAATYYC

SEQ ID NO: 66 - protein, 10 aa, Rabbit
FGGGTEVVVK

SEQ ID NO: 67 - DNA, 357 bp, Rabbit
cagtcgctggaggagtccgggggtcgcctggtcacgcctgggacacccctgacactcacctgcacagtctctggaatcgacctcagtagcgtgacaatgggctgggtccgccaggctccagggaaggggctggaatacatcggagtcataactactattcgtaccacatactacgcgagctgggtgaatggtcgattcaccatctcccaaacctcgaccacggtggatctgaaaatgaccagtctgacaaccgaggacacggccacctatttctgtgccagagatggtgatactactaatacttatggctactactttgacatctggggcccaggcaccctggtcaccgtctcctta

SEQ ID NO: 68 - DNA, 330 bp, Rabbit
gccgtcgtgatgacccagactccagcctctgtggaggtagctgtgggaggcacagtcaccattaagtgccaggccagtcagagcattagtagctacttagcctggtatcagcagaaaccagggcagcctcccaagctcctgatctacaaggcttccactctggcatctggggtcccatcgcggttcaaaggcagtggatctgggacacagttcactctcaccatcagcggcgtgcagtgtgacgatgctgccacttactactgtcaacagggttatactggtaataatgttgataatattttcggcggagggaccgaggtggtggtcaaa

SEQ ID NO: 69 - protein, 119 aa, Rabbit
QSLEESGGRLVTPGTPLTLTCTVSGIDLSSVTMGWVRQAPGKGLEYIGVITTIRTTYYASWVNGRFTISQTSTTVDLKMTSLTTEDTATYFCARDGDTTNTYGYYFDIWGPGTLVTVSL

SEQ ID NO: 70 - protein, 110 aa, Rabbit
AVVMTQTPASVEVAVGGTVTIKCQASQSISSYLAWYQQKPGQPPKLLIYKASTLASGVPSRFKGSGSGTQFTLTISGVQCDDAATYYCQQGYTGNNVDNIFGGGTEVVVK

SEQ ID NO: 71 - protein, 5 aa, Rabbit
SVTMG

SEQ ID NO: 72 - protein, 16 aa, Rabbit
VITTIRTTYYASWVNG

SEQ ID NO: 73 - protein, 14 aa, Rabbit
DGDTTNTYGYYFDI

SEQ ID NO: 74 - protein, 11 aa, Rabbit
QASQSISSYLA

SEQ ID NO: 75 - protein, 7 aa, Rabbit
KASTLAS

SEQ ID NO: 76 - protein, 12 aa, Rabbit
QQGYTGNNVDNI

SEQ ID NO: 77 - protein, 29 aa, Rabbit
QSLEESGGRLVTPGTPLTLTCTVSGIDLS

SEQ ID NO: 78 - protein, 14 aa, Rabbit
WVRQAPGKGLEYIG

SEQ ID NO: 79 - protein, 30 aa, Rabbit
RFTISQTSTTVDLKMTSLTTEDTATYFCAR

SEQ ID NO: 80 - protein, 11 aa, Rabbit
WGPGTLVTVSL

SEQ ID NO: 81 - protein, 23 aa, Rabbit
AVVMTQTPASVEVAVGGTVTIKC

SEQ ID NO: 82 - protein, 15 aa, Rabbit
WYQQKPGQPPKLLIY

SEQ ID NO: 83 - protein, 32 aa, Rabbit
GVPSRFKGSGSGTQFTLTISGVQCDDAATYYC

SEQ ID NO: 84 - protein, 10 aa, Rabbit
FGGGTEVVVK

SEQ ID NO: 85 - DNA, 330 bp, Rabbit
cagtcggtgggggagtccgggggtcgcctggtcacgcctgggacacccctgacactcacctgcacagtctctggaatcgacctcagtatctatccagtagactgggtccgccaggctccagggaaggggctggagtggatcggaatcattaataccaatggtaacacatactacgcgaactgggcgaaaggccgattcaccatctccaaaacctcgtcgaccacggtggatctgcaagtgaccagtctgacaaccgaggacacggccacctatttctgtgccagacactttgacttgtggggccaaggcaccctggtcaccgtctcctca

SEQ ID NO: 86 - DNA, 327 bp, Rabbit
caagtgctgacccagactgcatcccccgtgtctgcggctgttggaggcacagtcaccatcaattgccagtccagtcagactgtttataataacaacaacttagcctggtatcagcagaaatcagggcagcctcccaagctcctgatctatgattcatccaaactggaatctggggtcccatcgcggttcagcggcagtggatctgggacacagttcactctcaccatcagcgacctggagtgtgacgatgctgccacttactattgtgcaggcggttatggtggttggattgctgctttcggcggagggaccgaggtggtggtcaaa

SEQ ID NO: 87 - protein, 110 aa, Rabbit
QSVGESGGRLVTPGTPLTLTCTVSGIDLSIYPVDWVRQAPGKGLEWIGIINTNGNTYYANWAKGRFTISKTSSTTVDLQVTSLTTEDTATYFCARHFDLWGQGTLVTVSS

SEQ ID NO: 88 - protein, 109 aa, Rabbit
QVLTQTASPVSAAVGGTVTINCQSSQTVYNNNNLAWYQQKSGQPPKLLIYDSSKLESGVPSRFSGSGSGTQFTLTISDLECDDAATYYCAGGYGGWIAAFGGGTEVVVK

SEQ ID NO: 89 - protein, 5 aa, Rabbit
IYPVD

SEQ ID NO: 90 - protein, 16 aa, Rabbit
IINTNGNTYYANWAKG

SEQ ID NO: 91 - protein, 4 aa, Rabbit
HFDL

SEQ ID NO: 92 - protein, 13 aa, Rabbit
QSSQTVYNNNNLA

SEQ ID NO: 93 - protein, 7 aa, Rabbit
DSSKLES

SEQ ID NO: 94 - protein, 10 aa, Rabbit
AGGYGGWIAA

SEQ ID NO: 95 - protein, 29 aa, Rabbit
QSVGESGGRLVTPGTPLTLTCTVSGIDLS

SEQ ID NO: 96 - protein, 14 aa, Rabbit
WVRQAPGKGLEWIG

SEQ ID NO: 97 - protein, 31 aa, Rabbit
RFTISKTSSTTVDLQVTSLTTEDTATYFCAR

SEQ ID NO: 98 - protein, 11 aa, Rabbit
WGQGTLVTVSS

SEQ ID NO: 99 - protein, 22 aa, Rabbit
QVLTQTASPVSAAVGGTVTINC

SEQ ID NO: 100 - protein, 15 aa, Rabbit
WYQQKSGQPPKLLIY

SEQ ID NO: 101 - protein, 32 aa, Rabbit
GVPSRFSGSGSGTQFTLTISDLECDDAATYYC

SEQ ID NO: 102 - protein, 10 aa, Rabbit
FGGGTEVVVK

SEQ ID NO: 103 - DNA, 354 bp, Rabbit
cagtcgctggaggagtccgggggtcgcctggtcacgcctgggacacccctgacactcacctgcacagcctctggattgtccctcagcagctacgacatgaactgggtccgccaggctccagggaaggggctggaatacatcggattcattaatactcgtggtagcgcatactacgcgagctgggctacaggccgattcaccatctccagaacctcgaccacggtggatctgacaatgaccagtccgacaaccgaggacacggccacctatttctgtgccagagatattcctgattgggatattggtcatgtttttgatctctggggcccaggcaccctggtcaccgtctcctca

SEQ ID NO: 104 - DNA, 330 bp, Rabbit
gcctatgatatgacccagactccagcctctgtggaggtaactatgggaggcgcagtcaccatcaagtgccaggccagtcagagcattagcaatgaattatcctggtatcaacagaaaccagggcagcgtcccaggctcctgatttaccaggcttctgctctggcatctggggtcccatcgcggttcaaaggcagtggatatgggacacagttcactctcaccatcagcgacctggagtgtgccgatgctgccacttactactgtcaacagggttatactagtaataatattgtcaatgctttcggcggagggaccgaggtggtggtcaaa

SEQ ID NO: 105 - protein, 118 aa, Rabbit
QSLEESGGRLVTPGTPLTLTCTASGLSLSSYDMNWVRQAPGKGLEYIGFINTRGSAYYASWATGRFTISRTSTTVDLTMTSPTTEDTATYFCARDIPDWDIGHVFDLWGPGTLVTVSS

SEQ ID NO: 106 - protein, 110 aa, Rabbit
AYDMTQTPASVEVTMGGAVTIKCQASQSISNELSWYQQKPGQRPRLLIYQASALASGVPSRFKGSGYGTQFTLTISDLECADAATYYCQQGYTSNNIVNAFGGGTEVVVK

SEQ ID NO: 107 - protein, 5 aa, Rabbit
SYDMN

SEQ ID NO: 108 - protein, 16 aa, Rabbit
FINTRGSAYYASWATG

SEQ ID NO: 109 - protein, 13 aa, Rabbit
DIPDWDIGHVFDL

SEQ ID NO: 110 - protein, 11 aa, Rabbit
QASQSISNELS

SEQ ID NO: 111 - protein, 7 aa, Rabbit
QASALAS

SEQ ID NO: 112 - protein, 12 aa, Rabbit
QQGYTSNNIVNA

SEQ ID NO: 113 - protein, 29 aa, Rabbit
QSLEESGGRLVTPGTPLTLTCTASGLSLS

SEQ ID NO: 114 - protein, 14 aa, Rabbit
WVRQAPGKGLEYIG

SEQ ID NO: 115 - protein, 30 aa, Rabbit; identical to SEQ ID NO: 61

SEQ ID NO: 116 - protein, 11 aa, Rabbit
WGPGTLVTVSS

SEQ ID NO: 117 - protein, 23 aa, Rabbit
AYDMTQTPASVEVTMGGAVTIKC

SEQ ID NO: 118 - protein, 15 aa, Rabbit
WYQQKPGQRPRLLIY

SEQ ID NO: 119 - protein, 32 aa, Rabbit; identical to SEQ ID NO: 65

SEQ ID NO: 120 - protein, 10 aa, Rabbit
FGGGTEVVVK

SEQ ID NO: 121 - DNA, 369 bp, Rabbit
caggagcaagtggtggagtccgggggaggcctggtccagcctgagggatccctgacactcacctgcacagcctctggattcgacttcagtagcaatgcaatgtgctgggtccgccaggctccagggaaggggctggcgtggatcggatgcattggtactggtagtagtggtagcacttactacgcgagctgggcgagaggccgattcaccatctccaaaacctcgtcgaccacggtgactctgcaaatgaccagtctgacagccgcggacacggccacctatttctgtccgagagacattaatgcttatggtaatgatggttggtcttttaacttgtggggcccaggcaccctggtcaccgtctcgtca

SEQ ID NO: 122 - DNA, 330 bp, Rabbit
gcctatgatatgacccagactccagcctctgtggaggtagctgtgggaggcacagtcaccatcaagtgccaggccagtcagagtattgttggttggttatcctggtatcagcagaaacccgggcagcctcccaagctcctgatctacagggcatccactctggcatctggggtcccatcgcggttcaaaggcagtggatctgggacagagttcactctcaccatcagcgacctggagtgtgacgatgctgccacttactactgtcaacagggttatacttttaataatgttgataatcctttcggcggagggaccgaggtgttggtcaaa

SEQ ID NO: 123 - protein, 123 aa, Rabbit
QEQVVESGGGLVQPEGSLTLTCTASGFDFSSNAMCWVRQAPGKGLAWIGCIGTGSSGSTYYASWARGRFTISKTSSTTVTLQMTSLTAADTATYFCPRDINAYGNDGWSFNLWGPGTLVTVSS

SEQ ID NO: 124 - protein, 110 aa, Rabbit
AYDMTQTPASVEVAVGGTVTIKCQASQSIVGWLSWYQQKPGQPPKLLIYRASTLASGVPSRFKGSGSGTEFTLTISDLECDDAATYYCQQGYTFNNVDNPFGGGTEVLVK

SEQ ID NO: 125 - protein, 5 aa, Rabbit
SNAMC

SEQ ID NO: 126 - protein, 18 aa, Rabbit
CIGTGSSGSTYYASWARG

SEQ ID NO: 127 - protein, 14 aa, Rabbit
DINAYGNDGWSFNL

SEQ ID NO: 128 - protein, 11 aa, Rabbit
QASQSIVGWLS

SEQ ID NO: 129 - protein, 7 aa, Rabbit
RASTLAS

SEQ ID NO: 130 - protein, 12 aa, Rabbit
QQGYTFNNVDNP

SEQ ID NO: 131 - protein, 30 aa, Rabbit
QEQVVESGGGLVQPEGSLTLTCTASGFDFS

SEQ ID NO: 132 - protein, 14 aa, Rabbit
WVRQAPGKGLAWIG

SEQ ID NO: 133 - protein, 31 aa, Rabbit
RFTISKTSSTTVTLQMTSLTAADTATYFCPR

SEQ ID NO: 134 - protein, 11 aa, Rabbit
WGPGTLVTVSS

SEQ ID NO: 135 - protein, 23 aa, Rabbit
AYDMTQTPASVEVAVGGTVTIKC

SEQ ID NO: 136 - protein, 15 aa, Rabbit
WYQQKPGQPPKLLIY

SEQ ID NO: 137 - protein, 32 aa, Rabbit
GVPSRFKGSGSGTEFTLTISDLECDDAATYYC

SEQ ID NO: 138 - protein, 10 aa, Rabbit
FGGGTEVLVK

SEQ ID NO: 139 - DNA, 330 bp, Rabbit
cagtcggtgggggagtccgggggtcgcctggtcacgcctgggacacccctgacactcacctgcacagtctctggaatcgacctcagcgtctatccagtagactgggtccgccaggctccagggaaggggctggagtggatcgggatcattagtaccaatgggaacacatactacgcgaactgggcgaagggccgattcaccatctccaaaacctcgtcgaccacggtggatctgaaaatgaccagtctgacaaccgaggacacggccacctatttctgtgccagacactttgacttgtggggccaaggcactctggtcaccgtctcctca

SEQ ID NO: 140 - DNA, 327 bp, Rabbit
caagtgctgacccagactgcatcccccgtgtctgcggctgtgggaggcacagtcaccatcaattgccagtccagtcagactgtttataataacaataacttagcctggtatcagcagaaatcagggcagcctcccaagctcctgatttatgattcattcaaactggaatctggggtcccatcgcggttcagcggcagtggatctgggacacagttcactctcaccatcagcgacctggagtgtgacgatgctgccacttactattgtgcaggcggttatgctggttggattgctgctttcggcggagggaccgaggtggtggtcaaa

SEQ ID NO: 141 - protein, 110 aa, Rabbit
QSVGESGGRLVTPGTPLTLTCTVSGIDLSVYPVDWVRQAPGKGLEWIGIISTNGNTYYANWAKGRFTISKTSSTTVDLKMTSLTTEDTATYFCARHFDLWGQGTLVTVSS

SEQ ID NO: 142 - protein, 109 aa, Rabbit
QVLTQTASPVSAAVGGTVTINCQSSQTVYNNNNLAWYQQKSGQPPKLLIYDSFKLESGVPSRFSGSGSGTQFTLTISDLECDDAATYYCAGGYAGWIAAFGGGTEVVVK

SEQ ID NO: 143 - protein, 5 aa, Rabbit
VYPVD

SEQ ID NO: 144 - protein, 16 aa, Rabbit
IISTNGNTYYANWAKG

SEQ ID NO: 145 - protein, 4 aa, Rabbit
HFDL

SEQ ID NO: 146 - protein, 13 aa, Rabbit
QSSQTVYNNNNLA

SEQ ID NO: 147 - protein, 7 aa, Rabbit
DSFKLES

SEQ ID NO: 148 - protein, 10 aa, Rabbit
AGGYAGWIAA

SEQ ID NO: 149 - protein, 29 aa, Rabbit
QSVGESGGRLVTPGTPLTLTCTVSGIDLS

SEQ ID NO: 150 - protein, 14 aa, Rabbit
WVRQAPGKGLEWIG

SEQ ID NO: 151 - protein, 31 aa, Rabbit
RFTISKTSSTTVDLKMTSLTTEDTATYFCAR

SEQ ID NO: 152 - protein, 11 aa, Rabbit
WGQGTLVTVSS

SEQ ID NO: 153 - protein, 22 aa, Rabbit
QVLTQTASPVSAAVGGTVTINC

SEQ ID NO: 154 - protein, 15 aa, Rabbit
WYQQKSGQPPKLLIY

SEQ ID NO: 155 - protein, 32 aa, Rabbit; identical to SEQ ID NO: 101

SEQ ID NO: 156 - protein, 10 aa, Rabbit
FGGGTEVVVK

SEQ ID NO: 157 - DNA, 354 bp, Rabbit
cagtcgctggaggagtccgggggtcgcctggtcacgcctgggacacccctgacactcacctgcacagcctctggattgtccctcagcaggtacgacatgaactgggtccgccaggctccagggaaggggctggaatacatcggattcattaatactcgtggtagcacatactacgcgacctggactacaggccgattcaccatctccagaacctcgaccacggtggatctgacaatgaccagtccgacaaccgaggacacggccacctatttctgtgccagagatattcctgattgggatattggtcatgtttttgatctctggggcccaggcaccctggtcaccgtctcctca

SEQ ID NO: 158 - DNA, 330 bp, Rabbit
gcctatgatatgacccagactccagcctctgtgtctggagctgtgggaggcacagtcaccatcaattgccaggccagtcagagcattagcaatgaattatcctggtatcaacagaaaccagggcagcgtcccaggctcctgatttaccaggcttctactctggcatctggggtcccatcgcggttcaaaggcagtggatatgggacacagttcactctcaccatcagcgacctggagtgtgccgatgctgccacttactactgtcaacagggttatactagtaataatattgtcaatgctttcggcggagggaccgaggtggtggtcaaa

SEQ ID NO: 159 - protein, 118 aa, Rabbit
QSLEESGGRLVTPGTPLTLTCTASGLSLSRYDMNWVRQAPGKGLEYIGFINTRGSTYYATWTTGRFTISRTSTTVDLTMTSPTTEDTATYFCARDIPDWDIGHVFDLWGPGTLVTVSS

SEQ ID NO: 160 - protein, 110 aa, Rabbit
AYDMTQTPASVSGAVGGTVTINCQASQSISNELSWYQQKPGQRPRLLIYQASTLASGVPSRFKGSGYGTQFTLTISDLECADAATYYCQQGYTSNNIVNAFGGGTEVVVK

SEQ ID NO: 161 - protein, 5 aa, Rabbit
RYDMN

SEQ ID NO: 162 - protein, 16 aa, Rabbit
FINTRGSTYYATWTTG

SEQ ID NO: 163 - protein, 13 aa, Rabbit
DIPDWDIGHVFDL

SEQ ID NO: 164 - protein, 11 aa, Rabbit
QASQSISNELS

SEQ ID NO: 165 - protein, 7 aa, Rabbit
QASTLAS

SEQ ID NO: 166 - protein, 12 aa, Rabbit
QQGYTSNNIVNA

SEQ ID NO: 167 - protein, 29 aa, Rabbit
QSLEESGGRLVTPGTPLTLTCTASGLSLS

SEQ ID NO: 168 - protein, 14 aa, Rabbit
WVRQAPGKGLEYIG

SEQ ID NO: 169 - protein, 30 aa, Rabbit; identical to SEQ ID NO: 61

SEQ ID NO: 170 - protein, 11 aa, Rabbit
WGPGTLVTVSS

SEQ ID NO: 171 - protein, 23 aa, Rabbit
AYDMTQTPASVSGAVGGTVTINC

SEQ ID NO: 172 - protein, 15 aa, Rabbit
WYQQKPGQRPRLLIY

SEQ ID NO: 173 - protein, 32 aa, Rabbit; identical to SEQ ID NO: 65

SEQ ID NO: 174 - protein, 10 aa, Rabbit
FGGGTEVVVK

SEQ ID NO: 175 - DNA, 369 bp, Rabbit
caggagcaggtggtggagtccgggggaggcctggtccagcctgagggatccctgacactcacctgcacagcttctggattctcctttaataataatgcaatgtgctgggtccgccaggctccagggaaggggctggagtggatcggatgcattggtactgctgctagtggtaatagttactacgcgagctgggcgaaaggcagatttaccatctccagaacctcgtcgaccacggtgacgctacaaatgaccagtctgacagccgcggacacggccacctatttctgtccgagagacattaatgcttatggtaatgatggttggtcttttaagttgtggggcccaggcaccctggtcaccgtctcctca

SEQ ID NO: 176 - DNA, 330 bp, Rabbit
gcattcgaattgacccagactccatcctccgtggaggcagctgtgggaggcacagtcaccatcaagtgccaggccagtcagagcattaatggttggttatcctggtatcagcagaaaccagggcagcctcccaagctcctgatctatagggcatcgcctgtggcatctggggtcccatcgcggttcaaaggcagtggatctgggacagagtacactctcaccatcagcggcgtgcagtgtgacgatgctgccacttactactgtcaacagggttatacttttaataatgttgacgatcctttcggcggagggaccgaggtggtggtcaaa

SEQ ID NO: 177 - protein, 123 aa, Rabbit
QEQVVESGGGLVQPEGSLTLTCTASGFSFNNNAMCWVRQAPGKGLEWIGCIGTAASGNSYYASWAKGRFTISRTSSTTVTLQMTSLTAADTATYFCPRDINAYGNDGWSFKLWGPGTLVTVSS

SEQ ID NO: 178 - protein, 110 aa, Rabbit
AFELTQTPSSVEAAVGGTVTIKCQASQSINGWLSWYQQKPGQPPKLLIYRASPVASGVPSRFKGSGSGTEYTLTISGVQCDDAATYYCQQGYTFNNVDDPFGGGTEVVVK

SEQ ID NO: 179 - protein, 5 aa, Rabbit
NNAMC

SEQ ID NO: 180 - protein, 18 aa, Rabbit
CIGTAASGNSYYASWAKG

SEQ ID NO: 181 - protein, 14 aa, Rabbit
DINAYGNDGWSFKL

SEQ ID NO: 182 - protein, 11 aa, Rabbit
QASQSINGWLS

SEQ ID NO: 183 - protein, 7 aa, Rabbit
RASPVAS

SEQ ID NO: 184 - protein, 12 aa, Rabbit
QQGYTFNNVDDP

SEQ ID NO: 185 - protein, 30 aa, Rabbit
QEQVVESGGGLVQPEGSLTLTCTASGFSFN

SEQ ID NO: 186 - protein, 14 aa, Rabbit
WVRQAPGKGLEWIG

SEQ ID NO: 187 - protein, 31 aa, Rabbit
RFTISRTSSTTVTLQMTSLTAADTATYFCPR

SEQ ID NO: 188 - protein, 11 aa, Rabbit
WGPGTLVTVSS

SEQ ID NO: 189 - protein, 23 aa, Rabbit
AFELTQTPSSVEAAVGGTVTIKC

SEQ ID NO: 190 - protein, 15 aa, Rabbit
WYQQKPGQPPKLLIY

SEQ ID NO: 191 - protein, 32 aa, Rabbit
GVPSRFKGSGSGTEYTLTISGVQCDDAATYYC

SEQ ID NO: 192 - protein, 10 aa, Rabbit
FGGGTEVVVK

SEQ ID NO: 193 - DNA, 357 bp, Rabbit
cagtcgctggaggagtccgggggtcgcctggtcacgcctgggacacccctgacactcacctgcacagtctctggaatcgacctcagtagcgtgacaatgggctgggtccgccaggctccagggaaggggctggaatacatcggagtcattgttcctcgtggtgccacatactacgcgaactgggtgaaaggccgattcaccatctccaaaacctcgaccacggtggatctgaaaatgaccagtctgacaaccgaggacacggccacctatttctgtgccagagatggtgatactggtaatatttatggctattattttgacatctggggcccaggcaccctggtcaccgtctcctta

SEQ ID NO: 194 - DNA, 330 bp, Rabbit
gcctatgatatgacccagactccagcctccgtgtctgaacctgtgggaggcacagtcaccattaagtgccaggccagtcagagcattagcactgcattagcctggtatcagcagaaatcagggcagcgtcccaagctcctgatctacgatgcatggaaactggcatctggggtctcatcgcggttcaaaggcagtggatctgggacacagttcactctcaccatcagtggcgtgcagtgtgacgatgctgccacttactactgtcaacagggttatagtggtaataatgttgataatgttttcggcggagggaccgaggtggtggtcagc

SEQ ID NO: 195 - protein, 119 aa, Rabbit
QSLEESGGRLVTPGTPLTLTCTVSGIDLSSVTMGWVRQAPGKGLEYIGVIVPRGATYYANWVKGRFTISKTSTTVDLKMTSLTTEDTATYFCARDGDTGNIYGYYFDIWGPGTLVTVSL

SEQ ID NO: 196 - protein, 110 aa, Rabbit
AYDMTQTPASVSEPVGGTVTIKCQASQSISTALAWYQQKSGQRPKLLIYDAWKLASGVSSRFKGSGSGTQFTLTISGVQCDDAATYYCQQGYSGNNVDNVFGGGTEVVVS

SEQ ID NO: 197 - protein, 5 aa, Rabbit
SVTMG

SEQ ID NO: 198 - protein, 16 aa, Rabbit
VIVPRGATYYANWVKG

SEQ ID NO: 199 - protein, 14 aa, Rabbit
DGDTGNIYGYYFDI

SEQ ID NO: 200 - protein, 11 aa, Rabbit
QASQSISTALA

SEQ ID NO: 201 - protein, 7 aa, Rabbit
DAWKLAS

SEQ ID NO: 202 - protein, 12 aa, Rabbit
QQGYSGNNVDNV

SEQ ID NO: 203 - protein, 29 aa, Rabbit
QSLEESGGRLVTPGTPLTLTCTVSGIDLS

SEQ ID NO: 204 - protein, 14 aa, Rabbit
WVRQAPGKGLEYIG

SEQ ID NO: 205 - protein, 30 aa, Rabbit; identical to SEQ ID NO: 25

SEQ ID NO: 206 - protein, 11 aa, Rabbit
WGPGTLVTVSL

SEQ ID NO: 207 - protein, 23 aa, Rabbit
AYDMTQTPASVSEPVGGTVTIKC

SEQ ID NO: 208 - protein, 15 aa, Rabbit
WYQQKSGQRPKLLIY

SEQ ID NO: 209 - protein, 32 aa, Rabbit
GVSSRFKGSGSGTQFTLTISGVQCDDAATYYC

SEQ ID NO: 210 - protein, 10 aa, Rabbit
FGGGTEVVVS

SEQ ID NO: 211 - protein, 11 aa, Artificial Sequence
  Light CDR1 Consensus Sequence
  MISC_FEATURE (7)..(7): any amino acid
  MISC_FEATURE (8)..(8): any amino acid
  MISC_FEATURE (9)..(9): any amino acid
  MISC_FEATURE (11)..(11): any amino acid
QASQSIXXXLX

SEQ ID NO: 212 - protein, 11 aa, Artificial Sequence
  Light CDR1 Consensus Sequence
  MISC_FEATURE (7)..(7): Val or Ser or Thr or Asn
  MISC_FEATURE (8)..(8): Gly or Asn or Ser or Thr
  MISC_FEATURE (9)..(9): Trp or Glu or Tyr or Ala
  MISC_FEATURE (11)..(11): Ser or Ala
QASQSIXXXLX

SEQ ID NO: 213 - protein, 13 aa, Artificial Sequence
  Light CDR1 Consensus Sequence
  MISC_FEATURE (5)..(5): any amino acid
QSSQXVYNNNNLA

SEQ ID NO: 214 - protein, 13 aa, Artificial Sequence
  Light CDR1 Consensus Sequence
  MISC_FEATURE (5)..(5): Thr or Ser
QSSQXVYNNNNLA

SEQ ID NO: 215 - protein, 12 aa, Artificial Sequence
  Light CDR3 Consensus Sequence
  MISC_FEATURE (5)..(5): any amino acid
  MISC_FEATURE (6)..(6): any amino acid
  MISC_FEATURE (9)..(9): any amino acid
  MISC_FEATURE (10)..(10): any amino acid
  MISC_FEATURE (11)..(11): any amino acid
  MISC_FEATURE (12)..(12): any amino acid
QQGYXXNNXXXX

SEQ ID NO: 216 - protein, 12 aa, Artificial Sequence
  Light CDR3 Consensus Sequence
  MISC_FEATURE (5)..(5): Thr or Ser
  MISC_FEATURE (6)..(6): Phe or Gly or Ser
  MISC_FEATURE (9)..(9): Val or Ile
  MISC_FEATURE (10)..(10): Asp or Val
  MISC_FEATURE (11)..(11): Asn or Asp
  MISC_FEATURE (12)..(12): Pro or Ile or Val or Ala
QQGYXXNNXXXX

SEQ ID NO: 217 - protein, 10 aa, Artificial Sequence
  Light CDR3 Consensus Sequence
  MISC_FEATURE (5)..(5): any amino acid
  MISC_FEATURE (7)..(7): any amino acid
  MISC_FEATURE (10)..(10): any amino acid
AGGYXGXIAX

SEQ ID NO: 218 - protein, 10 aa, Artificial Sequence
  Light CDR3 Consensus Sequence
  MISC_FEATURE (5)..(5): Gly or Ala or Ser
  MISC_FEATURE (7)..(7): Trp or Val
  MISC_FEATURE (10)..(10): Ala or Ser
AGGYXGXIAX

(19) United States
(12) Patent Application Publication
(10) Pub. No.: US 2024/0052027 A1
(43) Pub. Date: Feb. 15, 2024

(54) TRPV1 EPITOPES AND ANTIBODIES

(71) Applicant: OBLIQUE THERAPEUTICS AB, Goteborg (SE)

(72) Inventors: Carolina TRKULJA, Hökerum (SE); Max DAVIDSON, Gothenburg (SE); Owe ORWAR, Hovås (SE)

(21) Appl. No.: 18/266,402

(22) PCT Filed: Dec. 10, 2021
(86) PCT No.: PCT/EP2021/085264

§ 371 Date: Jun. 9, 2023

(30) Foreign Application Priority Data
Dec. 10, 2020 (GB) 2019522.8

(51) Int. Cl. C07K 16/28

(52) U.S. Cl. CPC C07K 16/28; C07K 2317/76; C07K 2317/33; C07K 2317/34; C07K 2317/92

(57) ABSTRACT

The present invention relates to antibodies that bind to TRPV1. The invention also relates to certain epitopes of the protein TRPV1. The invention also relates to immunoconjugates and compositions comprising such antibodies. The invention also provides methods of producing such antibodies. The invention further provides the use of such antibodies for therapeutic purposes, for example in the treatment of pain.

### CROSS REFERENCE TO RELATED APPLICATIONS

This application claims the benefit of and priority to pending PCT Application Serial Number PCT/EP2021/085264, filed Dec. 10, 2021, which claims the benefit of and priority Great Britain Application Serial No. GB2019522.8, filed Dec. 10, 2020. The above-noted applications are incorporated by reference herein, in their entirety.

### REFERENCE TO SEQUENCE LISTING

A Sequence Listing is hereby incorporated by reference, in compliance with WIPO standard ST. 25. Said Sequence Listing has been filed as an electronic document via PatentCenter as a .txt file. The electronic document, created on Jun. 19, 2023, is entitled “10414-107US1”, and is 100,700 bytes in size.

### BACKGROUND OF THE INVENTION

This invention relates generally to the field of epitopes and antibodies, in particular epitopes of the protein TRPV1 and antibodies that bind to TRPV1. Such anti-TRPV1 antibodies have therapeutic uses, such as in the treatment of pain. Antibody-based compositions and methods and uses of the invention also extend to the use of conjugates and other therapeutic combinations, kits and methods.

TRPV1 (transient receptor potential vanilloid type 1) is an ion channel that is sensitive to noxious stimuli such as low pH, high temperatures (T≥42° C.), capsaicin, and inflammatory mediators. TRPV1 has been studied for almost two decades due to its involvement in pain sensation. Several attempts have been made to block the activity of the receptor as a new mode of action for pain therapies, but these attempts have not been successful.

It is well known in the art that capsaicin is an activator of TRPV1 and can cause pain. Capsaicin is routinely used in the art in models to assess TRPV1 activation and pain. Assessing inhibition of capsaicin-induced TRPV1 activation is a well-known model system for assessing the potential of agents to treat pain. Such model systems are described, for example, by Chen et al. (2011) (Chen et al., in TRP Channels, ed. MX, Z., 1-14, CRC Press/Taylor & Francis, 2011).

Due to the complexity of its activation mechanisms (as mentioned above it can be activated several stimuli including low pH, high temperatures (T≥42° C.), capsaicin, and inflammatory mediators), the majority of compounds targeting TRPV1 have produced adverse effects such as hyperthermia or loss of heat sensation.

For example, the compound AMG 517 is a potent TRPV1 small molecule antagonist developed by Amgen which inhibits capsaicin, pH and temperature activation of TRPV1. It was terminated after Phase 1 studies where it caused marked hyperthermia in subjects treated for molar extraction (Gawa, N. R. et al. Pain (2008), 136 (1-2), 202-210).

Another small molecule TRPV1 antagonist, Mavatrep, developed by Johnson & Johnson has shown efficacy in a Phase 1 b study of chronic pain including subjects with knee osteoarthritis, though several subjects reported side-effects of feeling hot and some experiencing minor thermal burns (Manitpisitkul P, et al. Scand. J. Pain (2018), 18(2):151-164).

Antibodies that bind to TRPV1 have previously been generated (Klionsky et al., The Journal of Pharmacology and Experimental Therapeutics (2006), Vol. 319 (1), pages 192-198), but these antibodies either do not inhibit capsaicin-induced activity of TRPV1 at all or, where an antibody is reported as inhibiting capsaicin-induced activity of TRPV1, an equivalent degree of inhibition of heat-induced activity of TRPV1 is also reported.

There is thus a need for alternative, preferably improved, treatments of pain. TRPV1 is a clinically and genetically validated target. Successful targeting of TRPV1 could provide a long sought-after solution to providing pain relief at its source.

In particular, identifying epitopes (or regions) on TRPV1, the targeting of which would lead to the inhibition of capsaicin activation of TRPV1 would be beneficial. This would guide the identification and generation of agents, such as antibodies, that bind to TRPV1 and reduce capsaicin activation of TRPV1. Identifying epitopes (or regions) on TRPV1, the targeting of which may lead to the preferential inhibition of capsaicin activation of TRPV1 as opposed to heat-induced activation of TRPV1 would be particularly beneficial. Agents, such as antibodies, that bind to TRPV1 and reduce capsaicin activation of TRPV1 without, or with reduced, heat-related side effects that have been observed with previous small molecule TRPV1 antagonists (e.g. AMG 517 and Mavatrep as described above) may be particularly beneficial.

The present inventors have addressed this need by identifying a region (or epitope) in the extracellular region of TRPV1 that is particularly useful to target, e.g. with antibodies, in order to inhibit capsaicin-induced activation of TRPV1, preferably to preferentially inhibit capsaicin-induced activation of TRPV1 as opposed to heat-induced activation of TRPV1. The extracellular region of TRPV1 is of course the region (or part) of TRPV1 that is exposed at (or accessible at, e.g. accessible by antibodies at) the extracellular side (or extracellular surface) of a cell when TRPV1 is expressed on (or at) the surface of a cell. The inventors have identified and generated isolated peptides that correspond to (or correspond essentially) to such a region (or epitope). The inventors have also used such isolated peptides to generate antibodies that bind to TRPV1. Antibodies have been generated by the present inventors that inhibit capsaicin activation of TRPV1. It has also been shown that antibodies that bind to a region in accordance with the invention may preferentially inhibit capsaicin activation of TRPV1 as opposed to heat-induced activation of TRPV1.

Thus, in one aspect, the present invention provides an isolated peptide comprising an amino acid sequence of SEQ ID NO:2 (OTV16) or a sequence substantially homologous thereto.

As discussed elsewhere herein, such isolated peptides may be used as antigenic peptides to generate antibodies that inhibit TRPV1 activation. Typically, such antibodies inhibit capsaicin-induced activation of TRPV1, preferably as opposed to heat-induced activation of TRPV1.

Unless otherwise clear from the context, references to “isolated peptides” or “peptides” of the invention may alternatively be considered references to “isolated epitopes” or “isolated antigenic epitopes”.

In some embodiments, the present invention provides an isolated peptide comprising an amino acid sequence of SEQ ID NO:2 (OTV16).

In some embodiments, the present invention provides an isolated peptide consisting of an amino acid sequence of SEQ ID NO:2 (OTV16) or a sequence substantially homologous thereto.

In some embodiments, the present invention provides an isolated peptide consisting of an amino acid sequence of SEQ ID NO:2 (OTV16).

In some embodiments, the isolated peptide may comprise one or more additional amino acids at the N- and/or C-terminus. In some preferred embodiments, the isolated peptide may comprise one or more additional amino acids at the N-terminus. In some preferred embodiments, the isolated peptide may comprise one or more additional amino acids at the C-terminus. In some preferred embodiments, the isolated peptide may comprise one or more additional amino acids at the N-terminus and at the C-terminus. In some preferred embodiments, the isolated peptide may comprise a cysteine (C) residue at the N- and/or at the C-terminus (or an additional cysteine (C) residue at the N- and/or at the C-terminus). In some embodiments, the isolated peptide may comprise a cysteine (C) residue at the N-terminus. In some embodiments, the isolated peptide may comprise a cysteine (C) residue at the C-terminus. In some preferred embodiments, the isolated peptide may comprise a cysteine (C) residue at the N- and C-terminus. The provision of a cysteine residue at a terminus of the isolated peptide can permit convenient attachment to a peptide carrier, e.g. as discussed elsewhere herein. The provision of cysteine residues at both the N- and C-termini provides a convenient means for cyclization of the peptide, if desired.

In some embodiments, the isolated peptide may comprise one or more additional modifications at the N- and/or C-terminus. For example, in some embodiments the isolated peptide may be C-terminally amidated. In some embodiments, the isolated peptide may have a modification (chemical group or linker) that may be used to attach (or link or connect) the peptide to a peptide carrier. In some embodiments, the modification (chemical group or linker) that may be used to attach (or link or connect) the peptide to a peptide carrier is a propargyl (Pra) group. A modification (chemical group or linker) that may be used to attach (or link or connect) the peptide to a peptide carrier may be at the N- and/or C-terminus. In some embodiments, a modification (chemical group or linker such as a propargyl group) that may be used to attach (or link or connect) the peptide to a peptide carrier is at the N-terminus of the isolated peptide.

In some embodiments, the present invention provides an isolated peptide comprising an amino acid sequence of SEQ ID NO:3 (OTV16) or a sequence substantially homologous thereto.

In some embodiments, the present invention provides an isolated peptide comprising an amino acid sequence of SEQ ID NO:3 (OTV16).

In some embodiments, the present invention provides an isolated peptide consisting of an amino acid sequence of SEQ ID NO:3 (OTV16) or a sequence substantially homologous thereto.

In some embodiments, the present invention provides an isolated peptide consisting of an amino acid sequence of SEQ ID NO:3 (OTV16).

In the context of the isolated peptide sequences of the invention, a sequence “substantially homologous” to a given amino acid sequence may be a sequence containing 1, 2, 3, 4, 5 or 6 (preferably 1, 2 or 3) amino acid substitutions or deletions or additions compared to the given amino acid sequence, or a sequence having at least 70% sequence identity to the given amino acid sequence, or a sequence having at least 6 consecutive amino acids of the given amino acid sequence. Other examples of “substantially homologous” sequences are described elsewhere herein in relation to amino acid sequences that are “substantially homologous” to isolated peptides and these examples of “substantially homologous” sequence are also applicable to the specific peptide sequences mentioned above.

In some preferred embodiments, amino acid sequences that are “substantially homologous” to isolated peptides are sequences having, or sequences comprising, a sequence that has, 1, 2, or 3 amino acid substitutions or additions or deletions (preferably 1 or 2, more preferably 1) compared with the amino acid sequence of the given isolated peptide.

Amino acid sequences that are “substantially homologous” to isolated peptides include sequences that comprise (or consist of) at least 5 or at least 6 consecutive amino acids of the isolated peptides (or comprise or consist of at least 7, at least 8, at least 9, at least 10, at least 11, at least 12 or at least 15 consecutive amino acids of the isolated peptide). Six amino acids is a typical length of peptide/protein sequence that is recognized or bound by an antibody.

Amino acid sequences that are “substantially homologous” to isolated peptides include sequences having, or sequences comprising a sequence that has, at least 50%, at least 55%, at least 60%, at least 65%, at least 70%, at least 75%, at least 80%, at least 85%, at least 90%, at least 95%, or at least 98% sequence identity to the given isolated peptide sequence. Sequence identities of at least 70%, at least 75%, at least 80%, at least 85%, at least 90%, at least 95%, or at least 98% are preferred.

Alterations in the amino acid sequences can be with conservative or non-conservative amino acids. Preferably said alterations are conservative amino acid substitutions.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

The term “substantially homologous” also includes modifications or chemical equivalents of the amino acid sequences of the present invention that perform substantially the same function as the proteins of the invention in substantially the same way. For example, any substantially homologous isolated peptide should typically retain the ability to act as peptide or epitope to (or against) which anti-antibodies which bind to TRPV1 can be generated (or raised).

Methods of carrying out the above described manipulation of amino acids (e.g. to generate “substantially homologous” sequences) are well known to a person skilled in the art.

In some embodiments, the isolated peptides do not contain any internal cysteine residues. By “internal” residue is meant a residue at a position other than the N-terminal and/or C-terminal residue. Thus, in some embodiments, a sequence that is “substantially homologous” to a given amino acid sequence does not have a cysteine (C) residue as the substituting or additional amino acid.

Homology (e.g. sequence identity) may be assessed by any convenient method. However, for determining the degree of homology (e.g. identity) between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al, Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

In some embodiments, the present invention provides an isolated peptide that comprises (or consists of) an elongated, truncated or cyclic version of an isolated peptide sequence disclosed herein (or a sequence substantially homologous thereto). In some embodiments, an isolated peptide may be elongated and cyclic (i.e. cyclized). In some embodiments, an isolated peptide may be truncated and cyclic (i.e. cyclized). Elongated, truncated and cyclic versions of peptides are discussed elsewhere herein.

An isolated peptide of the invention may comprise (or consist of) an elongated version of an isolated peptide sequence disclosed herein, or an elongated version of an amino acid sequence substantially homologous to an isolated peptide sequence disclosed herein. For example, one or more additional amino acids (e.g. at least 2, at least 3, at least 4, at least 5, at least 6, at least 7, at least 8 or at 9, at least 10, at least 15 or at least 20 amino acids, or 1-5 or 1-10 or 1-20 amino acids) may be present at one end or both ends of the isolated peptide sequence (or sequence substantially homologous thereto).

An isolated peptide of the invention may comprise (or consist of) a truncated version of an isolated peptide sequence disclosed herein, or a truncated version of an isolated peptide sequence disclosed herein. For example, one or more amino acids (e.g. at least 2, at least 3, at least 4, at least 5, at least 6, at least 7, at least 8 or at 9, at least 10 amino acids, or 1-5 or 1-10 amino acids) may be absent from one end or both ends of the isolated peptide sequence (or sequence substantially homologous thereto).

In some embodiments, isolated peptides may be at least 5, at least 6, at least 7, at least 8, at least 9 or at least 10 amino acids in length, for example 6 to 10, 6 to 12, 6 to 15, 6 to 20, 6 to 25, 6 to 30, 6 to 40, 6 to 50, 6 to 60, or 6 to 75 amino acids in length. Isolated peptides may be, for example, 5 to 7, 5 to 8, 5 to 9, 5 to 10, 5 to 15, 5 to 20, 5 to 25, 5 to 30, 5 to 40, 5 to 50, 5 to 60, 5 to 70, 5 to 75 amino acids in length. Isolated peptides may be, for example, 8 to 10, 8 to 15, 8 to 20, 8 to 25, 8 to 30, 8 to 40, 8 to 50, 8 to 60, 8 to 70, 8 to 75 amino acids in length.

In some embodiments, isolated peptides may be 50 amino acids in length, e.g. 45, 40, 35, 30, 25, 20, 15 or 0 amino acids in length (e.g. 5-10, 5-15, 5-20, 5-25, 5-30, 5-35, 5-40, 5-45, 5-50, 6-10, 6-15, 6-20, 6-25, 6-30, 6-35, 6-40, 6-45, 6-50, 8-10, 8-15, 8-20, 8-25, 8-30, 8-35, 8-40, 8-45, 8-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 25-30, 25-35 or 25-40, 25-45, 25-50, 30-35, 30-40, 30-45, 30-50, 35-40, 35-45, 35-50, 40-45, 40-50 or 45-50 amino acids in length).

In some embodiments, isolated peptides may be <39 amino acids in length, e.g. ≤38, ≤35, ≤30, ≤25, ≤20, ≤15, ≤10 amino acids in length (e.g. 5-10, 5-15, 5-20, 5-25, 5-30, 5-35, 5-38, 6-10, 6-15, 6-20, 6-25, 6-30, 6-35, 6-38, 8-10, 8-15, 8-20, 8-25, 8-30, 8-32, 8-35, 8-38, 10-15, 10-20, 10-25, 10-30, 10-32, 10-35, 10-38, 15-20, 15-25, 15-30, 15-32, 15-35, 15-38, 20-25, 20-30, 20-35, 25-30, 25-35 or 25-38 amino acids in length). In some embodiments, isolated peptides may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids in length.

In some embodiments, isolated peptides may be <24 amino acids in length, e.g. ≤23, ≤20, ≤15, ≤10 amino acids in length (e.g. 5-10, 5-15, 5-20, 5-23, 6-10, 6-15, 6-20, 6-23, 8-10, 8-15, 8-20, 8-23, 10-15, 10-20, 10-23, 15-20, 15-23 or 20-23 amino acids in length).

In some embodiments, the isolated peptides may be linear peptides (or linear epitopes).

In some embodiments, the isolated peptides may be conformational peptides (or conformational epitopes).

In some embodiments, the isolated peptides may be cyclic (or cyclized) peptides (or cyclic or cyclized epitopes).

In some preferred embodiments, isolated peptides based on SEQ ID NO:2 or SEQ ID NO:3 (or sequences substantially homologous thereto) are cyclic peptides.

In some embodiments, isolated peptides based on SEQ ID NO:2 or SEQ ID NO:3 (or sequences substantially homologous thereto) are <24 amino acids in length or <20 amino acids in length <15 amino acids in length <10 amino acids in length (e.g. are 5-23 or 8-23 or 10-23 or 15-23 or 16-23 or 20-23 or 5-10 or 8-10 or 10-15 or 5-15 or 8-15 or 10-15 or 5-20 or 8-20 or 10-20 or 16-20 amino acids in length). In some embodiments, isolated peptides based on SEQ ID NO:2 or SEQ ID NO:3 (or sequences substantially homologous thereto) are less than 39 amino acids in length, are less than 38 amino acids in length, or less than 37 amino acids in length, or less than 36 amino acids in length, or less than 35 amino acids in length, or less than 34 amino acids in length, or less than 33 amino acids in length (e.g. are 12-20 or 12-25 or 12-30 or 12-32 or 12-38 or 16-20 or 16-25 or 16-30 or 16-32 or 16-38 or 20-25 or 20-30 or 20-32 or 25-30 or 25-32 or 25-38 or 30-38 amino acids in length). In some embodiments, isolated peptides based on SEQ ID NO:2 or SEQ ID NO:3 (or sequences substantially homologous thereto) are 13-19 amino acids in length. In some embodiments, isolated peptides based on SEQ ID NO:2 or SEQ ID NO:3 (or sequences substantially homologous thereto) do not contain any internal cysteine residues. By “internal” residue is meant a residue at a position other than the N-terminal and/or C-terminal residue. In some embodiments, isolated peptides based on SEQ ID NO:2 or SEQ ID NO:3 (or sequences substantially homologous thereto) have an N-terminal cysteine residue and a C-terminal cysteine residue. In some embodiments, a further modification may be present at the N- and/or C-terminus (e.g. a propargyl group at the N-terminus and/or a C-terminal amide group).

In some embodiments, isolated peptides substantially homologous to SEQ ID NO:2 or SEQ ID NO:3 have 1, 2 or 3 amino acid substitutions, deletions or additions as compared to SEQ ID NO:2 or SEQ ID NO:3 themselves.

Preferably, isolated peptides based on SEQ ID NO:2 or SEQ ID NO:3 (or sequences substantially homologous thereto) are cyclic peptides. Preferably such peptides are cyclized via a disulphide bond between N- and C-terminal cysteine residues.

In another aspect, the present invention provides an isolated peptide comprising (or consisting of) an amino acid sequence of SEQ ID NO:4 (NHP-OTV16), or a sequence substantially homologous thereto. Substantially homologous sequences and/or other features and properties of such an isolated peptide may be as described elsewhere herein in connection with other isolated peptides of the invention.

In some embodiments, the present invention provides an isolated peptide comprising (or consisting of) an amino acid sequence of SEQ ID NO:5 (NHP-OTV16), or a sequence substantially homologous thereto.

In another aspect, the present invention provides an isolated peptide comprising (or consisting of) an amino acid sequence of SEQ ID NO:6 (rOTV16), or a sequence substantially homologous thereto. Substantially homologous sequences and/or other features and properties of such an isolated peptide may be as described elsewhere herein in connection with other isolated peptides of the invention.

In some embodiments, the present invention provides an isolated peptide comprising (or consisting of) an amino acid sequence of SEQ ID NO:7 (rOTV16), or a sequence substantially homologous thereto.

In another aspect, the present invention provides an isolated peptide comprising (or consisting of) an amino acid sequence of SEQ ID NO:8 (mOTV16), or a sequence substantially homologous thereto. Substantially homologous sequences and/or other features and properties of such an isolated peptide may be as described elsewhere herein in connection with other isolated peptides of the invention.

In some embodiments, the present invention provides an isolated peptide comprising (or consisting of) an amino acid sequence of SEQ ID NO:9 (mOTV16), or a sequence substantially homologous thereto.

In one aspect, the invention provides a conjugate comprising an isolated peptide comprising (or consisting of) SEQ ID NO:2 (or a sequence substantially homologous thereto) or comprising (or consisting of) SEQ ID NO:3 (or a sequence substantially homologous thereto) and (e.g. coupled to) a peptide carrier. Peptide carriers are described elsewhere herein. In a preferred embodiment the peptide carrier is KLH. In a preferred embodiment, the invention provides a conjugate comprising an isolated peptide consisting of SEQ ID NO:3 coupled to the peptide carrier KLH (preferably via the propargyl group of SEQ ID NO:3).

In another aspect, the invention provides a conjugate comprising an isolated peptide comprising (or consisting of) SEQ ID NO:4 (or a sequence substantially homologous thereto) or comprising (or consisting of) SEQ ID NO:5 (or a sequence substantially homologous thereto) and (e.g. coupled to) a peptide carrier. In another aspect, the invention provides a conjugate comprising an isolated peptide comprising (or consisting of) SEQ ID NO:6 (or a sequence substantially homologous thereto) or comprising (or consisting of) SEQ ID NO:7 (or a sequence substantially homologous thereto) and (e.g. coupled to) a peptide carrier. In another aspect, the invention provides a conjugate comprising an isolated peptide comprising (or consisting of) SEQ ID NO:8 (or a sequence substantially homologous thereto) or comprising (or consisting of) SEQ ID NO:9 (or a sequence substantially homologous thereto) and (e.g. coupled to) a peptide carrier. Peptide carriers are described elsewhere herein. In a preferred embodiment, the peptide carrier is KLH. In some embodiments, the invention provides a conjugate comprising an isolated peptide consisting of SEQ ID NO:5 coupled to the peptide carrier KLH (preferably via the propargyl group of SEQ ID NO:5). In some embodiments, the invention provides a conjugate comprising an isolated peptide consisting of SEQ ID NO:7 coupled to the peptide carrier KLH (preferably via the propargyl group of SEQ ID NO:7). In some embodiments, the invention provides a conjugate comprising an isolated peptide consisting of SEQ ID NO:9 coupled to the peptide carrier KLH (preferably via the propargyl group of SEQ ID NO:9).

Methods for synthesising peptides are well known in the art. A common technique used for preparing linear peptides (e.g. to be used for immunization and antibody generation) is Fmoc SPPS (Solid Phase Peptide Synthesis). In SPPS, small porous beads are treated with functional linkers on which peptide chains can be built using repeated cycles of wash-coupling-wash. The synthesized peptide is then released from the beads using chemical cleavage. For synthesis of cyclic peptides, common methods utilize cyclization by formation of a disulphide bridge (where the bridge is formed bridge by two cysteines of the peptide, e.g. one at the N-terminus and one at the C-terminus), or by formation of a “head-to-tail” bridge where the bridge consists of a typical peptide bond. Cyclic peptides can be formed on a solid support.

Other methods for synthesising peptides include using other chemical synthesis procedures, in vitro translation, or by introducing a suitable expression vector into cells.

Isolated peptides in accordance with the present invention of course do not include the full-length TRPV1 protein (i.e. wild-type TRPV1 protein), or any other full-length (wild-type) protein in the TRPV superfamily, or any other full-length (wild-type) proteins. Isolated peptides in accordance with the present invention thus do not include full-length SEQ ID NO:1. Isolated peptides in accordance with the present invention thus do not include full-length SEQ ID NO:10, full-length SEQ ID NO:11 or full-length SEQ ID NO:12.

Isolated peptides of the present invention, although corresponding to (or corresponding essentially to) regions (or epitopes) of the relevant full-length TRPV1 protein (e.g. as described elsewhere herein), do not themselves occur in nature (i.e. they do not have naturally occurring counterparts or do not occur in isolation in nature). Thus, the isolated peptides of the invention can be considered to be artificial peptides, or synthetic peptides, or man-made peptides, or non-native peptides.

A further aspect of the invention provides a conjugate. Typically the conjugate is configured to be used for the production of antibodies. The conjugate may comprise at least one isolated peptide as defined above coupled to (i.e. linked to or connected to or bonded to), or admixed with, a peptide carrier.

Thus, in one aspect, the invention provides a conjugate comprising an isolated peptide of the invention. Conjugates typically comprise an isolated peptide of the invention (preferably an isolated peptide comprising (or consisting of) SEQ ID NO:2 or SEQ ID NO:3, or a sequence substantially homologous thereto) and a peptide carrier, wherein said isolated peptide is coupled to, or admixed with, said peptide carrier. Peptide carriers typically enhance immunogenicity. This may be useful as, in some cases, short peptides which provide (or represent or correspond to) an antigenic epitope are, by themselves, too small to induce an immune response.

Peptide carriers are typically large macromolecules such as proteins, polysaccharides or polymeric amino acids. In some embodiments, the peptide carrier is selected from the group consisting of keyhole limpet hemocyanin (KLH), ovalbumin (OVA), serum albumins, polylysine and the like. KLH is typically preferred.

The coupling of an isolated peptide of the invention to a peptide carrier can, for example, be a covalent coupling or a disulphide bridge. In some embodiments, an isolated peptide of the invention may be provided with an (additional) cysteine residue at its N- or C-terminus (e.g. as described elsewhere herein). Such a cysteine residue may facilitate coupling of the isolated peptide to a peptide carrier (e.g. KLH). In some embodiments, the isolated peptide may have a modification (e.g. a chemical group such as a propargyl group) that permits coupling of the isolated peptide to a peptide carrier. In some embodiments, the isolated peptide is coupled to a peptide carrier via standard cross-linking agent (e.g. glutaraldehyde). Methods of linking isolated peptides to peptide carriers are well known in the art.

In some embodiments, isolated peptides (or conjugates) in accordance with the invention may be present in a solution or in a suspension. Thus, in one aspect the present invention provides a composition comprising an isolated peptide of the invention (preferably an isolated peptide comprising (or consisting of) SEQ ID NO:2 or SEQ ID NO:3, or a sequence substantially homologous thereto), and optionally an acceptable (e.g. a pharmaceutically acceptable) diluent, buffer, preservative and/or excipient.

In some embodiments, isolated peptides (or conjugates) may be present on (i.e. attached to or bound to) a solid support (e.g. a bead or microbead or plate or microtitre plate). Thus, in one aspect the present invention provides a solid support, having attached thereto (either directly or indirectly attached thereto) an isolated peptide (preferably an isolated peptide comprising (or consisting of) SEQ ID NO:2 or SEQ ID NO:3, or a sequence substantially homologous thereto) or conjugate of the invention.

Isolated peptides (and conjugates) of the invention (preferably isolated peptides comprising (or consisting of) SEQ ID NO:2 or SEQ ID NO:3, or a sequence substantially homologous thereto (and conjugates comprising such peptides)) are typically suitable for use in the identification (or generation or raising) of antibodies that bind to TRPV1 (preferably human TRPV1). For example, isolated peptides of the present invention are typically suitable for use as antigenic epitopes for the identification (or the generation or the raising) of antibodies. The identification (or the generation or the raising) of antibodies using isolated peptides of the invention may be done by any suitable means and the skilled person is familiar with suitable techniques (e.g. as discussed elsewhere herein). For example, isolated peptides (and conjugates) of the invention are typically suitable for use in the identification (or generation or raising) of polyclonal antibodies that bind to TRPV1 (e.g. polyclonal antibodies raised in an animal such as a rabbit that has been immunized with an isolated peptide (or conjugate) of the invention), or in in the identification (or generation or raising) of monoclonal antibodies using standard hybridoma technology or phage display. Put another way, isolated peptides (and conjugates) of the invention typically represent useful (or correspond to or correspond essentially to) regions (or epitopes) of TRPV1 to target with anti-TRPV1 antibodies.

In some embodiments, isolated peptides (and conjugates) of the invention (preferably isolated peptides comprising (or consisting of) SEQ ID NO:2 or SEQ ID NO:3, or a sequence substantially homologous thereto (and conjugates comprising such peptides)) are suitable for use in the identification (or generation or raising) of antibodies that bind to TRPV1 (preferably human TRPV1) and inhibit capsaicin-induced activation of TRPV1.

In some embodiments, isolated peptides (and conjugates) of the invention (preferably isolated peptides comprising (or consisting of) SEQ ID NO:2 or SEQ ID NO:3, or a sequence substantially homologous thereto (and conjugates comprising such peptides)) are suitable for use in the identification (or generation or raising) of antibodies that bind to TRPV1 (preferably human TRPV1) and do not significantly inhibit heat-induced activation of TRPV1.

In some embodiments, isolated peptides (and conjugates) of the invention (preferably isolated peptides comprising (or consisting of) SEQ ID NO:2 or SEQ ID NO:3, or a sequence substantially homologous thereto (and conjugates comprising such peptides)) are suitable for use in the identification (or generation or raising) of antibodies that bind to TRPV1 (preferably human TRPV1) and preferentially inhibit capsaicin-induced activation of TRPV1 as opposed to heat-induced activation of TRPV1.

Isolated peptides (and conjugates) of the invention comprising (or consisting of) SEQ ID NO:2 or SEQ ID NO:3, or a sequence substantially homologous thereto (and conjugates comprising such peptides) correspond to (or correspond essentially to) epitopes (or regions or portions) of TRPV1 (preferably human TRPV1, SEQ ID NO:1) that are positioned in the region of TRPV1 (preferably human TRPV1, SEQ ID NO:1) from amino acid residue 455 to 470.

Nucleic acid molecules comprising (or consisting of) nucleotide sequences that encode the isolated peptides of the present invention as defined herein, or nucleic acid molecules substantially homologous thereto, form yet further aspects of the invention.

The term “substantially homologous” as used herein in connection with an nucleic acid sequence includes sequences having at least 65%, 70% or 75%, preferably at least 80%, and even more preferably at least 85%, 90%, 95%, 96%, 97%, 98% or 99%, sequence identity to the starting nucleic acid sequence.

The term “nucleic acid sequence” or “nucleic acid molecule” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

In another aspect, the present invention provides a composition comprising an isolated peptide (or conjugate) of the invention. Such compositions may further comprise (e.g. be in admixture with) a suitable diluent, carrier, excipient and/or preservative (e.g. a pharmaceutically acceptable diluent, carrier, excipient and/or preservative).

As indicated above, isolated peptides (and conjugates) of the invention (preferably isolated peptides comprising (or consisting of) SEQ ID NO:2 or SEQ ID NO:3, or a sequence substantially homologous thereto (and conjugates comprising such peptides)) are typically suitable for use in the identification (or generation or raising) of antibodies that bind to TRPV1 (preferably human TRPV1) and inhibit capsaicin-induced activation of TRPV1, preferably as opposed to heat-induced activation of TRPV1.

In one aspect, the present invention provides an antibody, for example an isolated antibody, which binds to (or specifically recognises or specifically binds to) TRPV1, wherein said antibody binds to an epitope of TRPV1 that is in the region of TRPV1 defined by amino acid residues 455-470 of TRPV1 (SEQ ID NO:1).

In preferred embodiments, the TRPV1 (transient receptor potential vanilloid type 1) is human TRPV1 (hTRPV1). The amino acid sequence of human TRPV1 is set forth herein as SEQ ID NO:1.

In accordance with this aspect of the invention, antibodies of the invention bind to an epitope of TRPV1 that is located in an extracellular region of the TRPV1 protein. More specifically, antibodies bind to an epitope of TRPV1 in the region of TRPV1 defined by amino acid residues 455-470 of TRPV1 (SEQ ID NO:1). In some embodiments, the entire epitope bound lies within this region of TRPV1. In some embodiments, at least one amino acid of the epitope bound lies within this region of TRPV1.

Alternatively viewed, in accordance with this aspect of the invention, antibodies of the present invention typically bind to an epitope of TRPV1 in the region of TRPV1 consisting of amino acid residues 455-470 of TRPV1 (SEQ ID NO:1).

Alternatively viewed, in accordance with this aspect of the invention, antibodies of the present invention typically bind to TRPV1 in the region of TRPV1 consisting of amino acid residues 455-470 of TRPV1 (SEQ ID NO:1).

In preferred embodiments, antibodies of the invention bind to (or are capable of binding to) an isolated peptide or conjugate of the invention. Preferred isolated peptides and conjugates and preferred groups of isolated peptides and conjugates are defined elsewhere herein. In preferred embodiments, antibodies of the invention bind to a preferred isolated peptide or conjugate as described elsewhere herein. The ability of an antibody to bind to an isolated peptide can be assessed by any appropriate means and skilled person is familiar with suitable methods (e.g. an ELISA assay to assess whether or not a given isolated peptide can compete with full length TRPV1 for antibody binding).

Although preferred antibodies of the invention bind to (or are capable of binding to) an isolated peptide or conjugate of the invention, they of course also typically bind to full-length (or wild-type or native) TRPV1 (preferably human TRPV1).

In some embodiments, the full-length (or wild-type or native) TRPV1 (preferably human TRPV1) is TRPV1 that is expressed on cells, preferably on mammalian cells (e.g. on adherent Chinese hamster ovary (CHO) cells). In some embodiments, antibodies of the present invention are capable of binding to TRPV1 that is heterologously expressed on cells (e.g. CHO cells). In some embodiments, antibodies of the present invention are capable of binding to TRPV1 that is expressed on cells, wherein the TRPV1 is expressed from an inducible expression system (such as a tetracycline regulated expression system e.g. as described in the Example section herein). TRPV1 is typically expressed on (or at) the surface of cells. The binding of an antibody of the invention to TRPV1 may be assessed by any suitable means, and the skilled person will be familiar with suitable methods (e.g. flow cytometry (such as FACS) or immunocytochemistry or using a functional assay e.g. as described elsewhere herein).

The isolated peptides of the present invention correspond to, or correspond essentially to, certain regions or epitopes of full-length TRPV1 (preferably human TRPV1, SEQ ID NO:1).

The isolated peptide of SEQ ID NO:2 (an OTV16 peptide) corresponds to residues 455-470 of human TRPV1 (SEQ ID NO:1). The isolated peptide of SEQ ID NO:3 (an OTV16 peptide) corresponds essentially to residues 455-470 of human TRPV1 (SEQ ID NO:1).

Unless otherwise clear from the context, by “corresponds to” is meant that the amino sequence (SEQ ID NO:) of the isolated peptide matches the amino acid sequence of the stated region or epitope of human TRPV1 (SEQ ID NO:1). Unless otherwise clear from the context, by “corresponds essentially to” is meant that the amino acid sequence of the isolated peptide (SEQ ID NO:) is identifiable as being based on (or derived from or a modified form of) the sequence of the stated region or epitope of human TRPV1 (SEQ ID NO:1). For example, an isolated peptide having a sequence that “corresponds essentially to” the stated region or epitope of human TRPV1 (SEQ ID NO:1) typically has one or more (e.g. 1, 2, 3, 4 or 5, preferably 1, 2 or 3) amino acid substitutions, additions or deletions as compared to an isolated peptide that corresponds to (i.e. exactly corresponds to) the sequence of the stated region or epitope of human TRPV1 (SEQ ID NO:1). Thus, an isolated peptide having a sequence that “corresponds essentially to” the stated region or epitope of human TRPV1 (SEQ ID NO:1) may be considered to be a “substantially homologous” isolated peptide sequence as defined elsewhere herein.

In some embodiments, an antibody of the invention comprises at least one heavy chain variable region that comprises three CDRs and at least one light chain variable region that comprises three CDRs, wherein said light chain variable region comprises a variable light (VL) CDR1 that has the amino acid sequence of SEQ ID NO:211 (preferably SEQ ID NO:212) or a sequence substantially homologous thereto, or comprises a variable light (VL) CDR1 that has the amino acid sequence of SEQ ID NO:213 (preferably SEQ ID NO:214) or a sequence substantially homologous thereto.

In some embodiments, an antibody of the invention comprises at least one heavy chain variable region that comprises three CDRs and at least one light chain variable region that comprises three CDRs, wherein said light chain variable region comprises a variable light (VL) CDR1 that has the amino acid sequence of SEQ ID NO:211 (preferably SEQ ID NO:212) or a sequence substantially homologous thereto.

In some embodiments, an antibody of the invention comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:107, SEQ ID NO:161, SEQ ID NO:35, SEQ ID NO:71, SEQ ID NO:197, SEQ ID NO:53, SEQ ID NO: 125 or SEQ ID NO:179, or a sequence substantially homologous thereto,
(b) a VH CDR2 that has the amino acid sequence of SEQ ID NO:108, SEQ ID NO:162, SEQ ID NO:36, SEQ ID NO:72, SEQ ID NO:198, SEQ ID NO:54, SEQ ID NO:126 or SEQ ID NO:180, or a sequence substantially homologous thereto, and
(c) a VH CDR3 that has the amino acid sequence of SEQ ID NO:109, SEQ ID NO:163, SEQ ID NO:37, SEQ ID NO:73, SEQ ID NO:199, SEQ ID NO:55, SEQ ID NO:127 or SEQ ID NO:181, or a sequence substantially homologous thereto; and/or (preferably “and”)

wherein said light chain variable region comprises:
(d) a variable light (VL) CDR1 that has the amino acid sequence of SEQ ID NO:211 or preferably SEQ ID NO:212, or a sequence substantially homologous thereto,
(e) a VL CDR2 that has the amino acid sequence of SEQ ID NO:111 or SEQ ID NO:165, SEQ ID NO:39, SEQ ID NO:75, SEQ ID NO:201, SEQ ID NO:57, SEQ ID NO:129 or SEQ ID NO:183, or a sequence substantially homologous thereto, and
(f) a VL CDR3 that has the amino acid sequence of SEQ ID NO:112 or SEQ ID NO:166, SEQ ID NO:40, SEQ ID NO:76, SEQ ID NO:202, SEQ ID NO:58, SEQ ID NO:130 or SEQ ID NO:184, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VL CDR1 has or comprises an amino acid sequence of SEQ ID NO:211 (Q A S Q S I X₇ X₈ X₉ L X₁₁). In these embodiments, X₇, X₈, X₉ and X₁₁ can be any amino acid. Preferably, X₇ is V or S or T or N; X₈ is G or N or S or T; X₉ is W or E or Y or A; and X₁₁ is S or A. Thus, a preferred VL CDR1 has or comprises the amino acid sequence of SEQ ID NO:212. For example, preferred VL CDR1 sequences of this embodiment have or comprise SEQ ID NOs:110, 164, 38, 74, 200, 56, 128 or 182.

In some preferred embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:211 or preferably SEQ ID NO:212, or a sequence substantially homologous thereto, and wherein
(i) said light chain variable region comprises a VL CDR2 that has the amino acid sequence of SEQ ID NO:111 or a sequence substantially homologous thereto and a VL CDR3 that has the amino acid sequence of SEQ ID NO:112 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto; or
(ii) said light chain variable region comprises a VL CDR2 that has the amino acid sequence of SEQ ID NO:165 or a sequence substantially homologous thereto and a VL CDR3 that has the amino acid sequence of SEQ ID NO:166 or a sequence substantially homologous thereto, and/or (preferably “and”) said heavy chain variable region comprises a VH CDR1 that has the amino acid sequence of SEQ ID NO:161 or a sequence substantially homologous thereto, a VH CDR2 that has the amino acid sequence of SEQ ID NO:162 or a sequence substantially homologous thereto, and a VH CDR3 that has the amino acid sequence of SEQ ID NO:163 or a sequence substantially homologous thereto; or
(iii) said light chain variable region comprises a VL CDR2 that has the amino acid sequence of SEQ ID NO:39 or a sequence substantially homologous thereto and a VL CDR3 that has the amino acid sequence of SEQ ID NO:40 or a sequence substantially homologous thereto, and/or (preferably “and”) said heavy chain variable region comprises a VH CDR1 that has the amino acid sequence of SEQ ID NO:35 or a sequence substantially homologous thereto, a VH CDR2 that has the amino acid sequence of SEQ ID NO:36 or a sequence substantially homologous thereto, and a VH CDR3 that has the amino acid sequence of SEQ ID NO:37 or a sequence substantially homologous thereto; or
(iv) said light chain variable region comprises a VL CDR2 that has the amino acid sequence of SEQ ID NO:75 or a sequence substantially homologous thereto and a VL CDR3 that has the amino acid sequence of SEQ ID NO:76 or a sequence substantially homologous thereto, and/or (preferably “and”) said heavy chain variable region comprises a VH CDR1 that has the amino acid sequence of SEQ ID NO:71 or a sequence substantially homologous thereto, a VH CDR2 that has the amino acid sequence of SEQ ID NO:72 or a sequence substantially homologous thereto, and a VH CDR3 that has the amino acid sequence of SEQ ID NO:73 or a sequence substantially homologous thereto; or
(v) said light chain variable region comprises a VL CDR2 that has the amino acid sequence of SEQ ID NO:201 or a sequence substantially homologous thereto and a VL CDR3 that has the amino acid sequence of SEQ ID NO:202 or a sequence substantially homologous thereto, and/or (preferably “and”) said heavy chain variable region comprises a VH CDR1 that has the amino acid sequence of SEQ ID NO:197 or a sequence substantially homologous thereto, a VH CDR2 that has the amino acid sequence of SEQ ID NO:198 or a sequence substantially homologous thereto, and a VH CDR3 that has the amino acid sequence of SEQ ID NO:199 or a sequence substantially homologous thereto; or
(vi) said light chain variable region comprises a VL CDR2 that has the amino acid sequence of SEQ ID NO:57 or a sequence substantially homologous thereto and a VL CDR3 that has the amino acid sequence of SEQ ID NO:58 or a sequence substantially homologous thereto, and/or (preferably “and”) said heavy chain variable region comprises a VH CDR1 that has the amino acid sequence of SEQ ID NO:53 or a sequence substantially homologous thereto, a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(vii) said light chain variable region comprises a VL CDR2 that has the amino acid sequence of SEQ ID NO:129 or a sequence substantially homologous thereto and a VL CDR3 that has the amino acid sequence of SEQ ID NO:130 or a sequence substantially homologous thereto, and/or (preferably “and”) said heavy chain variable region comprises a VH CDR1 that has the amino acid sequence of SEQ ID NO:125 or a sequence substantially homologous thereto, a VH CDR2 that has the amino acid sequence of SEQ ID NO:126 or a sequence substantially homologous thereto, and a VH CDR3 that has the amino acid sequence of SEQ ID NO:127 or a sequence substantially homologous thereto; or
(viii) said light chain variable region comprises a VL CDR2 that has the amino acid sequence of SEQ ID NO:183 or a sequence substantially homologous thereto and a VL CDR3 that has the amino acid sequence of SEQ ID NO:184 or a sequence substantially homologous thereto, and/or (preferably “and”) said heavy chain variable region comprises a VH CDR1 that has the amino acid sequence of SEQ ID NO:179 or a sequence substantially homologous thereto, a VH CDR2 that has the amino acid sequence of SEQ ID NO:180 or a sequence substantially homologous thereto, and a VH CDR3 that has the amino acid sequence of SEQ ID NO:181 or a sequence substantially homologous thereto.

In some embodiments of the invention, for antibodies that comprise a VL CDR1 that has the amino acid sequence that is substantially homologous to SEQ ID NO: 110, 164, 38, 74, 200, 56, 128 and 182, the VL CDR1 may have an amino acid sequence of (i.e. that meets the criteria of) SEQ ID NO:211 or SEQ ID NO:212.

In some embodiments, an antibody of the invention comprises at least one heavy chain variable region that comprises three CDRs and at least one light chain variable region that comprises three CDRs, wherein said heavy chain variable region comprises a variable light (VL) CDR1 that has the amino acid sequence of SEQ ID NO:213 (preferably SEQ ID NO:214) or a sequence substantially homologous thereto.

In some embodiments, an antibody of the invention comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:89, SEQ ID NO:143 or SEQ ID NO:17, or a sequence substantially homologous thereto,
(b) a VH CDR2 that has the amino acid sequence of SEQ ID NO:90, SEQ ID NO:144 or SEQ ID NO:18, or a sequence substantially homologous thereto, and
(c) a VH CDR3 that has the amino acid sequence of SEQ ID NO:91, SEQ ID NO:145 or SEQ ID NO:19, or a sequence substantially homologous thereto; and/or (preferably “and”)

wherein said light chain variable region comprises:
(d) a variable light (VL) CDR1 that has the amino acid sequence of SEQ ID NO:213 or preferably SEQ ID NO:214, or a sequence substantially homologous thereto,
(e) a VL CDR2 that has the amino acid sequence of SEQ ID NO:93, SEQ ID NO: 147 or SEQ ID NO:21, or a sequence substantially homologous thereto, and
(f) a VL CDR3 that has the amino acid sequence of SEQ ID NO:94, SEQ ID NO:148 or SEQ ID NO:22, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VL CDR1 has or comprises an amino acid sequence of SEQ ID NO:213 (Q S S Q X₅ V Y N N N N L A). In these embodiments, X₅ can be any amino acid. Preferably, X₅ is T or S. Thus, a preferred VL CDR1 has or comprises the amino acid sequence of SEQ ID NO:214. For example, preferred VL CDR1 sequences of this embodiment have or comprise SEQ ID NOs:92, 146 or 20.

In some preferred embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:213 or preferably SEQ ID NO:214, or a sequence substantially homologous thereto, and wherein
(i) said light chain variable region comprises a VL CDR2 that has the amino acid sequence of SEQ ID NO:93 or a sequence substantially homologous thereto and a VL CDR3 that has the amino acid sequence of SEQ ID NO:94 or a sequence substantially homologous thereto, and/or (preferably “and”) said heavy chain variable region comprises a VH CDR1 that has the amino acid sequence of SEQ ID NO:89 or a sequence substantially homologous thereto, a VH CDR2 that has the amino acid sequence of SEQ ID NO:90 or a sequence substantially homologous thereto, and a VH CDR3 that has the amino acid sequence of SEQ ID NO:91 or a sequence substantially homologous thereto; or
(ii) said light chain variable region comprises a VL CDR2 that has the amino acid sequence of SEQ ID NO:147 or a sequence substantially homologous thereto and a VL CDR3 that has the amino acid sequence of SEQ ID NO:148 or a sequence substantially homologous thereto, and/or (preferably “and”) said heavy chain variable region comprises a VH CDR1 that has the amino acid sequence of SEQ ID NO:143 or a sequence substantially homologous thereto, a VH CDR2 that has the amino acid sequence of SEQ ID NO:144 or a sequence substantially homologous thereto, and a VH CDR3 that has the amino acid sequence of SEQ ID NO:145 or a sequence substantially homologous thereto; or
(iii) said light chain variable region comprises a VL CDR2 that has the amino acid sequence of SEQ ID NO:21 or a sequence substantially homologous thereto and a VL CDR3 that has the amino acid sequence of SEQ ID NO:22 or a sequence substantially homologous thereto, and/or (preferably “and”) said heavy chain variable region comprises a VH CDR1 that has the amino acid sequence of SEQ ID NO:17 or a sequence substantially homologous thereto, a VH CDR2 that has the amino acid sequence of SEQ ID NO:18 or a sequence substantially homologous thereto, and a VH CDR3 that has the amino acid sequence of SEQ ID NO:19 or a sequence substantially homologous thereto.

In some embodiments of the invention, for antibodies that comprise a VL CDR1 that has the amino acid sequence that is substantially homologous to SEQ ID NO:92, 146 or 20, the VL CDR1 may have an amino acid sequence of (i.e. that meets the criteria of) SEQ ID NO:213 or SEQ ID NO:214.

In some embodiments, an antibody of the invention comprises at least one heavy chain variable region that comprises three CDRs and at least one light chain variable region that comprises three CDRs, wherein said light chain variable region comprises a variable light (VL) CDR3 that has the amino acid sequence of SEQ ID NO:215 (preferably SEQ ID NO:216) or a sequence substantially homologous thereto, or comprises a variable light (VL) CDR3 that has the amino acid sequence of SEQ ID NO:217 (preferably SEQ ID NO:218) or a sequence substantially homologous thereto.

In some embodiments, an antibody of the invention comprises at least one heavy chain variable region that comprises three CDRs and at least one light chain variable region that comprises three CDRs, wherein said light chain variable region comprises a variable light (VL) CDR3 that has the amino acid sequence of SEQ ID NO:215 (preferably SEQ ID NO:216) or a sequence substantially homologous thereto.

In some embodiments, an antibody of the invention comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:107, SEQ ID NO:161, SEQ ID NO:71, SEQ ID NO:197, SEQ ID NO:53, SEQ ID NO: 125 or SEQ ID NO:179, or a sequence substantially homologous thereto,
(b) a VH CDR2 that has the amino acid sequence of SEQ ID NO:108, SEQ ID NO:162, SEQ ID NO:72, SEQ ID NO:198, SEQ ID NO:54, SEQ ID NO:126 or SEQ ID NO:180, or a sequence substantially homologous thereto, and
(c) a VH CDR3 that has the amino acid sequence of SEQ ID NO:109, SEQ ID NO:163, SEQ ID NO:73, SEQ ID NO:199, SEQ ID NO:55, SEQ ID NO:127 or SEQ ID NO:181, or a sequence substantially homologous thereto; and/or (preferably “and”)

wherein said light chain variable region comprises:
(d) a variable light (VL) CDR1 that has the amino acid sequence of SEQ ID NO:110, SEQ ID NO:164, SEQ ID NO:74, SEQ ID NO:200, SEQ ID NO:56, SEQ ID NO:128 or SEQ ID NO:182, or a sequence substantially homologous thereto,
(e) a VL CDR2 that has the amino acid sequence of SEQ ID NO:111 or SEQ ID NO:165, SEQ ID NO:75, SEQ ID NO:201, SEQ ID NO:57, SEQ ID NO:129 or SEQ ID NO:183, or a sequence substantially homologous thereto, and
(f) a VL CDR3 that has the amino acid sequence of SEQ ID NO:215 or preferably SEQ ID NO:216, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VL CDR3 has or comprises an amino acid sequence of SEQ ID NO:215 (Q Q G Y X₅ X₆ N N X₉ X₁₀ X₁₁ X₁₂). In these embodiments, X₅, X₆, X₉, X₁₀, X₁₁, and X₁₂ can be any amino acid. Preferably, X₅ is T or S; X₆ is F or G or S; X₉ is V or I; X₁₀ is D or V; X₁₁ is N or D; X₁₂ is P or I or V or A. Thus, a preferred VL CDR3 has or comprises the amino acid sequence of SEQ ID NO:216. For example, preferred VL CDR3 sequences of this embodiment have or comprise SEQ ID NOs:112, 166, 76, 202, 58, 130 or 184.

In some preferred embodiments, the antibody comprises at least one heavy chain variable region that comprises three CDRs and at least one light chain variable region that comprises three CDRs wherein said light chain variable region comprises a VL CDR3 that has the amino acid sequence of SEQ ID NO:215 or preferably SEQ ID NO:216, or a sequence substantially homologous thereto, and wherein
(i) said light chain variable region comprises a VL CDR2 that has the amino acid sequence of SEQ ID NO:111 or a sequence substantially homologous thereto and a VL CDR1 that has the amino acid sequence of SEQ ID NO:110 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto; or
(ii) said light chain variable region comprises a VL CDR2 that has the amino acid sequence of SEQ ID NO:165 or a sequence substantially homologous thereto and a VL CDR1 that has the amino acid sequence of SEQ ID NO:164 or a sequence substantially homologous thereto, and/or (preferably “and”) said heavy chain variable region comprises a VH CDR1 that has the amino acid sequence of SEQ ID NO:161 or a sequence substantially homologous thereto, a VH CDR2 that has the amino acid sequence of SEQ ID NO:162 or a sequence substantially homologous thereto, and a VH CDR3 that has the amino acid sequence of SEQ ID NO:163 or a sequence substantially homologous thereto; or
(iii) said light chain variable region comprises a VL CDR2 that has the amino acid sequence of SEQ ID NO:75 or a sequence substantially homologous thereto and a VL CDR1 that has the amino acid sequence of SEQ ID NO:74 or a sequence substantially homologous thereto, and/or (preferably “and”) said heavy chain variable region comprises a VH CDR1 that has the amino acid sequence of SEQ ID NO:71 or a sequence substantially homologous thereto, a VH CDR2 that has the amino acid sequence of SEQ ID NO:72 or a sequence substantially homologous thereto, and a VH CDR3 that has the amino acid sequence of SEQ ID NO:73 or a sequence substantially homologous thereto; or
(iv) said light chain variable region comprises a VL CDR2 that has the amino acid sequence of SEQ ID NO:201 or a sequence substantially homologous thereto and a VL CDR1 that has the amino acid sequence of SEQ ID NO:200 or a sequence substantially homologous thereto, and/or (preferably “and”) said heavy chain variable region comprises a VH CDR1 that has the amino acid sequence of SEQ ID NO:197 or a sequence substantially homologous thereto, a VH CDR2 that has the amino acid sequence of SEQ ID NO:198 or a sequence substantially homologous thereto, and a VH CDR3 that has the amino acid sequence of SEQ ID NO:199 or a sequence substantially homologous thereto; or
(v) said light chain variable region comprises a VL CDR2 that has the amino acid sequence of SEQ ID NO:57 or a sequence substantially homologous thereto and a VL CDR1 that has the amino acid sequence of SEQ ID NO:56 or a sequence substantially homologous thereto, and/or (preferably “and”) said heavy chain variable region comprises a VH CDR1 that has the amino acid sequence of SEQ ID NO:53 or a sequence substantially homologous thereto, a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(vi) said light chain variable region comprises a VL CDR2 that has the amino acid sequence of SEQ ID NO:129 or a sequence substantially homologous thereto and a VL CDR1 that has the amino acid sequence of SEQ ID NO:128 or a sequence substantially homologous thereto, and/or (preferably “and”) said heavy chain variable region comprises a VH CDR1 that has the amino acid sequence of SEQ ID NO:125 or a sequence substantially homologous thereto, a VH CDR2 that has the amino acid sequence of SEQ ID NO:126 or a sequence substantially homologous thereto, and a VH CDR3 that has the amino acid sequence of SEQ ID NO:127 or a sequence substantially homologous thereto; or
(vii) said light chain variable region comprises a VL CDR2 that has the amino acid sequence of SEQ ID NO:183 or a sequence substantially homologous thereto and a VL CDR1 that has the amino acid sequence of SEQ ID NO:182 or a sequence substantially homologous thereto, and/or (preferably “and”) said heavy chain variable region comprises a VH CDR1 that has the amino acid sequence of SEQ ID NO:179 or a sequence substantially homologous thereto, a VH CDR2 that has the amino acid sequence of SEQ ID NO:180 or a sequence substantially homologous thereto, and a VH CDR3 that has the amino acid sequence of SEQ ID NO:181 or a sequence substantially homologous thereto.

In some embodiments of the invention, for antibodies that comprise a VL CDR3 that has the amino acid sequence that is substantially homologous to SEQ ID NO:112, 166, 76, 202, 58, 130 or 184, the VL CDR3 may have an amino acid sequence of (i.e. that meets the criteria of) SEQ ID NO:215 or SEQ ID NO:216.

In some embodiments, an antibody of the invention comprises at least one heavy chain variable region that comprises three CDRs and at least one light chain variable region that comprises three CDRs, wherein said heavy chain variable region comprises a variable light (VL) CDR3 that has the amino acid sequence of SEQ ID NO:217 (preferably SEQ ID NO:218) or a sequence substantially homologous thereto.

In some embodiments, an antibody of the invention comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:89, SEQ ID NO:143 or SEQ ID NO:17, or a sequence substantially homologous thereto,
(b) a VH CDR2 that has the amino acid sequence of SEQ ID NO:90, SEQ ID NO:144 or SEQ ID NO:18, or a sequence substantially homologous thereto, and
(c) a VH CDR3 that has the amino acid sequence of SEQ ID NO:91, SEQ ID NO:145 or SEQ ID NO:19, or a sequence substantially homologous thereto; and/or (preferably “and”)

wherein said light chain variable region comprises:
(d) a variable light (VL) CDR1 that has the amino acid sequence of SEQ ID NO:92, SEQ ID NO:146 or SEQ ID NO:20, or a sequence substantially homologous thereto,
(e) a VL CDR2 that has the amino acid sequence of SEQ ID NO:93, SEQ ID NO: 147 or SEQ ID NO:21, or a sequence substantially homologous thereto, and
(f) a VL CDR3 that has the amino acid sequence of SEQ ID NO:217 or preferably SEQ ID NO:218, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments of the present invention, the VL CDR3 has or comprises an amino acid sequence of SEQ ID NO:217 (A G G Y X₅ G X₇ I A X₁₀). In these embodiments, X₅, X₇ and X₁₀ can be any amino acid. Preferably, X₅ is G or A or S; X₇ is W or V; and X₁₀ is A or S. Thus, a preferred VL CDR3 has or comprises the amino acid sequence of SEQ ID NO:218. For example, preferred VL CDR3 sequences of this embodiment have or comprise SEQ ID NOs:94, 148 or 22.

In some preferred embodiments, the antibody comprises at least one heavy chain variable region that comprises three CDRs and at least one light chain variable region that comprises three CDRs wherein said light chain variable region comprises a VL CDR3 that has the amino acid sequence of SEQ ID NO:217 or preferably SEQ ID NO:218, or a sequence substantially homologous thereto, and wherein
(i) said light chain variable region comprises a VL CDR2 that has the amino acid sequence of SEQ ID NO:93 or a sequence substantially homologous thereto and a VL CDR1 that has the amino acid sequence of SEQ ID NO:92 or a sequence substantially homologous thereto, and/or (preferably “and”) said heavy chain variable region comprises a VH CDR1 that has the amino acid sequence of SEQ ID NO:89 or a sequence substantially homologous thereto, a VH CDR2 that has the amino acid sequence of SEQ ID NO:90 or a sequence substantially homologous thereto, and a VH CDR3 that has the amino acid sequence of SEQ ID NO:91 or a sequence substantially homologous thereto; or
(ii) said light chain variable region comprises a VL CDR2 that has the amino acid sequence of SEQ ID NO:147 or a sequence substantially homologous thereto and a VL CDR1 that has the amino acid sequence of SEQ ID NO:146 or a sequence substantially homologous thereto, and/or (preferably “and”) said heavy chain variable region comprises a VH CDR1 that has the amino acid sequence of SEQ ID NO:143 or a sequence substantially homologous thereto, a VH CDR2 that has the amino acid sequence of SEQ ID NO:144 or a sequence substantially homologous thereto, and a VH CDR3 that has the amino acid sequence of SEQ ID NO:145 or a sequence substantially homologous thereto; or
(iii) said light chain variable region comprises a VL CDR2 that has the amino acid sequence of SEQ ID NO:21 or a sequence substantially homologous thereto and a VL CDR1 that has the amino acid sequence of SEQ ID NO:20 or a sequence substantially homologous thereto, and/or (preferably “and”) said heavy chain variable region comprises a VH CDR1 that has the amino acid sequence of SEQ ID NO:17 or a sequence substantially homologous thereto, a VH CDR2 that has the amino acid sequence of SEQ ID NO:18 or a sequence substantially homologous thereto, and a VH CDR3 that has the amino acid sequence of SEQ ID NO:19 or a sequence substantially homologous thereto.

In some embodiments of the invention, for antibodies that comprise a VL CDR3 that has the amino acid sequence that is substantially homologous to SEQ ID NO:94, 148 or 22, the VL CDR3 may have an amino acid sequence of (i.e. that meets the criteria of) SEQ ID NO:217 or SEQ ID NO:218.

In some embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:211 (preferably SEQ ID NO:212) or a sequence substantially homologous thereto, and comprises a VL CDR3 that has the amino acid sequence of SEQ ID NO:215 (preferably SEQ ID NO:216) or a sequence substantially homologous thereto.

In some preferred embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:211 (preferably SEQ ID NO:212) or a sequence substantially homologous thereto, and comprises a VL CDR3 that has the amino acid sequence of SEQ ID NO:215 (preferably SEQ ID NO:216) or a sequence substantially homologous thereto, and wherein
(i) said light chain variable region comprises a VL CDR2 that has the amino acid sequence of SEQ ID NO:111 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto; or
(ii) said light chain variable region comprises a VL CDR2 that has the amino acid sequence of SEQ ID NO:165 or a sequence substantially homologous thereto, and/or (preferably “and”) said heavy chain variable region comprises a VH CDR1 that has the amino acid sequence of SEQ ID NO:161 or a sequence substantially homologous thereto, a VH CDR2 that has the amino acid sequence of SEQ ID NO:162 or a sequence substantially homologous thereto, and a VH CDR3 that has the amino acid sequence of SEQ ID NO:163 or a sequence substantially homologous thereto; or
(iii) said light chain variable region comprises a VL CDR2 that has the amino acid sequence of SEQ ID NO:75 or a sequence substantially homologous thereto, and/or (preferably “and”) said heavy chain variable region comprises a VH CDR1 that has the amino acid sequence of SEQ ID NO:71 or a sequence substantially homologous thereto, a VH CDR2 that has the amino acid sequence of SEQ ID NO:72 or a sequence substantially homologous thereto, and a VH CDR3 that has the amino acid sequence of SEQ ID NO:73 or a sequence substantially homologous thereto; or
(iv) said light chain variable region comprises a VL CDR2 that has the amino acid sequence of SEQ ID NO:201 or a sequence substantially homologous thereto, and/or (preferably “and”) said heavy chain variable region comprises a VH CDR1 that has the amino acid sequence of SEQ ID NO:197 or a sequence substantially homologous thereto, a VH CDR2 that has the amino acid sequence of SEQ ID NO:198 or a sequence substantially homologous thereto, and a VH CDR3 that has the amino acid sequence of SEQ ID NO:199 or a sequence substantially homologous thereto; or
(v) said light chain variable region comprises a VL CDR2 that has the amino acid sequence of SEQ ID NO:57 or a sequence substantially homologous thereto, and/or (preferably “and”) said heavy chain variable region comprises a VH CDR1 that has the amino acid sequence of SEQ ID NO:53 or a sequence substantially homologous thereto, a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(vi) said light chain variable region comprises a VL CDR2 that has the amino acid sequence of SEQ ID NO:129 or a sequence substantially homologous thereto, and/or (preferably “and”) said heavy chain variable region comprises a VH CDR1 that has the amino acid sequence of SEQ ID NO:125 or a sequence substantially homologous thereto, a VH CDR2 that has the amino acid sequence of SEQ ID NO:126 or a sequence substantially homologous thereto, and a VH CDR3 that has the amino acid sequence of SEQ ID NO:127 or a sequence substantially homologous thereto; or
(vii) said light chain variable region comprises a VL CDR2 that has the amino acid sequence of SEQ ID NO:183 or a sequence substantially homologous thereto, and/or (preferably “and”) said heavy chain variable region comprises a VH CDR1 that has the amino acid sequence of SEQ ID NO:179 or a sequence substantially homologous thereto, a VH CDR2 that has the amino acid sequence of SEQ ID NO:180 or a sequence substantially homologous thereto, and a VH CDR3 that has the amino acid sequence of SEQ ID NO:181 or a sequence substantially homologous thereto.

In some preferred embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:213 (preferably SEQ ID NO:214) or a sequence substantially homologous thereto, and comprises a VL CDR3 that has the amino acid sequence of SEQ ID NO:217 (preferably SEQ ID NO:218) or a sequence substantially homologous thereto.

In some preferred embodiments, the antibody comprises at least one heavy chain variable region that comprises three CDRs and at least one light chain variable region that comprises three CDRs wherein said light chain variable region comprises a VL CDR1 that has the amino acid sequence of SEQ ID NO:213 (preferably SEQ ID NO:214) or a sequence substantially homologous thereto, and comprises a VL CDR3 that has the amino acid sequence of SEQ ID NO:217 (preferably SEQ ID NO:218) or a sequence substantially homologous thereto, and wherein
(i) said light chain variable region comprises a VL CDR2 that has the amino acid sequence of SEQ ID NO:21 or a sequence substantially homologous thereto, and/or (preferably “and”) said heavy chain variable region comprises a VH CDR1 that has the amino acid sequence of SEQ ID NO:17 or a sequence substantially homologous thereto, a VH CDR2 that has the amino acid sequence of SEQ ID NO:18 or a sequence substantially homologous thereto, and a VH CDR3 that has the amino acid sequence of SEQ ID NO:19 or a sequence substantially homologous thereto; or
(ii) said light chain variable region comprises a VL CDR2 that has the amino acid sequence of SEQ ID NO:93 or a sequence substantially homologous thereto, and/or (preferably “and”) said heavy chain variable region comprises a VH CDR1 that has the amino acid sequence of SEQ ID NO:89 or a sequence substantially homologous thereto, a VH CDR2 that has the amino acid sequence of SEQ ID NO:90 or a sequence substantially homologous thereto, and a VH CDR3 that has the amino acid sequence of SEQ ID NO:91 or a sequence substantially homologous thereto; or
(iii) said light chain variable region comprises a VL CDR2 that has the amino acid sequence of SEQ ID NO:147 or a sequence substantially homologous thereto, and/or (preferably “and”) said heavy chain variable region comprises a VH CDR1 that has the amino acid sequence of SEQ ID NO:143 or a sequence substantially homologous thereto, a VH CDR2 that has the amino acid sequence of SEQ ID NO:144 or a sequence substantially homologous thereto, and a VH CDR3 that has the amino acid sequence of SEQ ID NO:145 or a sequence substantially homologous thereto.

In some preferred embodiments, an antibody of the invention comprises at least one heavy chain variable region that comprises three CDRs and at least one light chain variable region that comprises three CDRs wherein said heavy chain variable region comprises a VH CDR1 that has the amino acid sequence of SEQ ID NO:53 or a sequence substantially homologous thereto, and wherein
(i) said heavy chain variable region comprises a VH CDR2 that has the amino acid sequence of SEQ ID NO:54 or a sequence substantially homologous thereto and a VH CDR3 that has the amino acid sequence of SEQ ID NO:55 or a sequence substantially homologous thereto, and/or (preferably “and”) said light chain variable region comprises a VL CDR1 that has the amino acid sequence of SEQ ID NO:56 or a sequence substantially homologous thereto, a VL CDR2 that has the amino acid sequence of SEQ ID NO:57 or a sequence substantially homologous thereto, and a VL CDR3 that has the amino acid sequence of SEQ ID NO:58 or a sequence substantially homologous thereto; or
(ii) said heavy chain variable region comprises a VH CDR2 that has the amino acid sequence of SEQ ID NO:108 or a sequence substantially homologous thereto and a VH CDR3 that has the amino acid sequence of SEQ ID NO:109 or a sequence substantially homologous thereto, and/or (preferably “and”) said light chain variable region comprises a VL CDR1 that has the amino acid sequence of SEQ ID NO:110 or a sequence substantially homologous thereto, a VL CDR2 that has the amino acid sequence of SEQ ID NO:111 or a sequence substantially homologous thereto, and a VL CDR3 that has the amino acid sequence of SEQ ID NO:112 or a sequence substantially homologous thereto.

In some preferred embodiments, an antibody of the invention comprises at least one heavy chain variable region that comprises three CDRs and at least one light chain variable region that comprises three CDRs wherein said heavy chain variable region comprises a VH CDR1 that has the amino acid sequence of SEQ ID NO:71 or a sequence substantially homologous thereto, and wherein
(i) said heavy chain variable region comprises a VH CDR2 that has the amino acid sequence of SEQ ID NO:72 or a sequence substantially homologous thereto and a VH CDR3 that has the amino acid sequence of SEQ ID NO:73 or a sequence substantially homologous thereto, and/or (preferably “and”) said light chain variable region comprises a VL CDR1 that has the amino acid sequence of SEQ ID NO:74 or a sequence substantially homologous thereto, a VL CDR2 that has the amino acid sequence of SEQ ID NO:75 or a sequence substantially homologous thereto, and a VL CDR3 that has the amino acid sequence of SEQ ID NO:76 or a sequence substantially homologous thereto; or
(ii) said heavy chain variable region comprises a VH CDR2 that has the amino acid sequence of SEQ ID NO:198 or a sequence substantially homologous thereto and a VH CDR3 that has the amino acid sequence of SEQ ID NO:199 or a sequence substantially homologous thereto, and/or (preferably “and”) said light chain variable region comprises a VL CDR1 that has the amino acid sequence of SEQ ID NO:200 or a sequence substantially homologous thereto, a VL CDR2 that has the amino acid sequence of SEQ ID NO:201 or a sequence substantially homologous thereto, and a VL CDR3 that has the amino acid sequence of SEQ ID NO:202 or a sequence substantially homologous thereto.

In some preferred embodiments, an antibody of the invention comprises at least one heavy chain variable region that comprises three CDRs and at least one light chain variable region that comprises three CDRs wherein said heavy chain variable region comprises a VH CDR3 that has the amino acid sequence of SEQ ID NO:91 or a sequence substantially homologous thereto, and wherein
(i) said heavy chain variable region comprises a VH CDR2 that has the amino acid sequence of SEQ ID NO:90 or a sequence substantially homologous thereto and a VH CDR1 that has the amino acid sequence of SEQ ID NO:89 or a sequence substantially homologous thereto, and/or (preferably “and”) said light chain variable region comprises a VL CDR1 that has the amino acid sequence of SEQ ID NO:92 or a sequence substantially homologous thereto, a VL CDR2 that has the amino acid sequence of SEQ ID NO:93 or a sequence substantially homologous thereto, and a VL CDR3 that has the amino acid sequence of SEQ ID NO:94 or a sequence substantially homologous thereto; or
(ii) said heavy chain variable region comprises a VH CDR2 that has the amino acid sequence of SEQ ID NO:144 or a sequence substantially homologous thereto and a VH CDR1 that has the amino acid sequence of SEQ ID NO:143 or a sequence substantially homologous thereto, and/or (preferably “and”) said light chain variable region comprises a VL CDR1 that has the amino acid sequence of SEQ ID NO:146 or a sequence substantially homologous thereto, a VL CDR2 that has the amino acid sequence of SEQ ID NO:147 or a sequence substantially homologous thereto, and a VL CDR3 that has the amino acid sequence of SEQ ID NO:148 or a sequence substantially homologous thereto; or
(iii) said heavy chain variable region comprises a VH CDR2 that has the amino acid sequence of SEQ ID NO:18 or a sequence substantially homologous thereto and a VH CDR1 that has the amino acid sequence of SEQ ID NO:17 or a sequence substantially homologous thereto, and/or (preferably “and”) said light chain variable region comprises a VL CDR1 that has the amino acid sequence of SEQ ID NO:21 or a sequence substantially homologous thereto, a VL CDR2 that has the amino acid sequence of SEQ ID NO:22 or a sequence substantially homologous thereto, and a VL CDR3 that has the amino acid sequence of SEQ ID NO:23 or a sequence substantially homologous thereto.

In some preferred embodiments, an antibody of the invention comprises at least one heavy chain variable region that comprises three CDRs and at least one light chain variable region that comprises three CDRs wherein said heavy chain variable region comprises a VH CDR3 that has the amino acid sequence of SEQ ID NO:109 or a sequence substantially homologous thereto, and wherein
(i) said heavy chain variable region comprises a VH CDR2 that has the amino acid sequence of SEQ ID NO:108 or a sequence substantially homologous thereto and a VH CDR1 that has the amino acid sequence of SEQ ID NO:107 or a sequence substantially homologous thereto, and/or (preferably “and”) said light chain variable region comprises a VL CDR1 that has the amino acid sequence of SEQ ID NO:110 or a sequence substantially homologous thereto, a VL CDR2 that has the amino acid sequence of SEQ ID NO:111 or a sequence substantially homologous thereto, and a VL CDR3 that has the amino acid sequence of SEQ ID NO:112 or a sequence substantially homologous thereto; or
(ii) said heavy chain variable region comprises a VH CDR2 that has the amino acid sequence of SEQ ID NO:162 or a sequence substantially homologous thereto and a VH CDR1 that has the amino acid sequence of SEQ ID NO:161 or a sequence substantially homologous thereto, and/or (preferably “and”) said light chain variable region comprises a VL CDR1 that has the amino acid sequence of SEQ ID NO:164 or a sequence substantially homologous thereto, a VL CDR2 that has the amino acid sequence of SEQ ID NO:165 or a sequence substantially homologous thereto, and a VL CDR3 that has the amino acid sequence of SEQ ID NO:166 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR1 that has the amino acid sequence of SEQ ID NO:110 or a sequence substantially homologous thereto, and wherein
(i) said light chain variable region comprises a VL CDR2 that has the amino acid sequence of SEQ ID NO:111 or a sequence substantially homologous thereto and a VL CDR3 that has the amino acid sequence of SEQ ID NO:112 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto; or
(ii) said light chain variable region comprises a VL CDR2 that has the amino acid sequence of SEQ ID NO:165 or a sequence substantially homologous thereto and a VL CDR3 that has the amino acid sequence of SEQ ID NO:166 or a sequence substantially homologous thereto, and/or (preferably “and”) said heavy chain variable region comprises a VH CDR1 that has the amino acid sequence of SEQ ID NO:161 or a sequence substantially homologous thereto, a VH CDR2 that has the amino acid sequence of SEQ ID NO:162 or a sequence substantially homologous thereto, and a VH CDR3 that has the amino acid sequence of SEQ ID NO:163 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR1 that has the amino acid sequence of SEQ ID NO:38 or a sequence substantially homologous thereto, and wherein
(i) said light chain variable region comprises a VL CDR2 that has the amino acid sequence of SEQ ID NO:39 or a sequence substantially homologous thereto and a VL CDR3 that has the amino acid sequence of SEQ ID NO:40 or a sequence substantially homologous thereto, and/or (preferably “and”) said heavy chain variable region comprises a VH CDR1 that has the amino acid sequence of SEQ ID NO:35 or a sequence substantially homologous thereto, a VH CDR2 that has the amino acid sequence of SEQ ID NO:36 or a sequence substantially homologous thereto, and a VH CDR3 that has the amino acid sequence of SEQ ID NO:37 or a sequence substantially homologous thereto; or
(ii) said light chain variable region comprises a VL CDR2 that has the amino acid sequence of SEQ ID NO:75 or a sequence substantially homologous thereto and a VL CDR3 that has the amino acid sequence of SEQ ID NO:76 or a sequence substantially homologous thereto, and/or (preferably “and”) said heavy chain variable region comprises a VH CDR1 that has the amino acid sequence of SEQ ID NO:71 or a sequence substantially homologous thereto, a VH CDR2 that has the amino acid sequence of SEQ ID NO:72 or a sequence substantially homologous thereto, and a VH CDR3 that has the amino acid sequence of SEQ ID NO:73 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR1 that has the amino acid sequence of SEQ ID NO:92 or a sequence substantially homologous thereto, and wherein
(i) said light chain variable region comprises a VL CDR2 that has the amino acid sequence of SEQ ID NO:93 or a sequence substantially homologous thereto and a VL CDR3 that has the amino acid sequence of SEQ ID NO:94 or a sequence substantially homologous thereto, and/or (preferably “and”) said heavy chain variable region comprises a VH CDR1 that has the amino acid sequence of SEQ ID NO:89 or a sequence substantially homologous thereto, a VH CDR2 that has the amino acid sequence of SEQ ID NO:90 or a sequence substantially homologous thereto, and a VH CDR3 that has the amino acid sequence of SEQ ID NO:91 or a sequence substantially homologous thereto; or
(ii) said light chain variable region comprises a VL CDR2 that has the amino acid sequence of SEQ ID NO:147 or a sequence substantially homologous thereto and a VL CDR3 that has the amino acid sequence of SEQ ID NO:148 or a sequence substantially homologous thereto, and/or (preferably “and”) said heavy chain variable region comprises a VH CDR1 that has the amino acid sequence of SEQ ID NO:143 or a sequence substantially homologous thereto, a VH CDR2 that has the amino acid sequence of SEQ ID NO:144 or a sequence substantially homologous thereto, and a VH CDR3 that has the amino acid sequence of SEQ ID NO:145 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR2 that has the amino acid sequence of SEQ ID NO:57 or a sequence substantially homologous thereto, and wherein
(i) said light chain variable region comprises a VL CDR1 that has the amino acid sequence of SEQ ID NO:56 or a sequence substantially homologous thereto and a VL CDR3 that has the amino acid sequence of SEQ ID NO:58 or a sequence substantially homologous thereto, and/or (preferably “and”) said heavy chain variable region comprises a VH CDR1 that has the amino acid sequence of SEQ ID NO:53 or a sequence substantially homologous thereto, a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(ii) said light chain variable region comprises a VL CDR1 that has the amino acid sequence of SEQ ID NO:110 or a sequence substantially homologous thereto and a VL CDR3 that has the amino acid sequence of SEQ ID NO:112 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR2 that has the amino acid sequence of SEQ ID NO:21 or a sequence substantially homologous thereto, and wherein
(i) said light chain variable region comprises a VL CDR1 that has the amino acid sequence of SEQ ID NO:20 or a sequence substantially homologous thereto and a VL CDR3 that has the amino acid sequence of SEQ ID NO:22 or a sequence substantially homologous thereto, and/or (preferably “and”) said heavy chain variable region comprises a VH CDR1 that has the amino acid sequence of SEQ ID NO:17 or a sequence substantially homologous thereto, a VH CDR2 that has the amino acid sequence of SEQ ID NO:18 or a sequence substantially homologous thereto, and a VH CDR3 that has the amino acid sequence of SEQ ID NO:19 or a sequence substantially homologous thereto; or
(ii) said light chain variable region comprises a VL CDR1 that has the amino acid sequence of SEQ ID NO:92 or a sequence substantially homologous thereto and a VL CDR3 that has the amino acid sequence of SEQ ID NO:94 or a sequence substantially homologous thereto, and/or (preferably “and”) said heavy chain variable region comprises a VH CDR1 that has the amino acid sequence of SEQ ID NO:89 or a sequence substantially homologous thereto, a VH CDR2 that has the amino acid sequence of SEQ ID NO:90 or a sequence substantially homologous thereto, and a VH CDR3 that has the amino acid sequence of SEQ ID NO:91 or a sequence substantially homologous thereto.

In some preferred embodiments, an antibody of the invention comprises at least one heavy chain variable region that comprises three CDRs and at least one light chain variable region that comprises three CDRs wherein said light chain variable region comprises a VL CDR3 that has the amino acid sequence of SEQ ID NO:58 or a sequence substantially homologous thereto, and wherein
(i) said light chain variable region comprises a VL CDR1 that has the amino acid sequence of SEQ ID NO:56 or a sequence substantially homologous thereto and a VL CDR2 that has the amino acid sequence of SEQ ID NO:57 or a sequence substantially homologous thereto, and/or (preferably “and”) said heavy chain variable region comprises a VH CDR1 that has the amino acid sequence of SEQ ID NO:53 or a sequence substantially homologous thereto, a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(ii) said light chain variable region comprises a VL CDR1 that has the amino acid sequence of SEQ ID NO:164 or a sequence substantially homologous thereto and a VL CDR2 that has the amino acid sequence of SEQ ID NO:165 or a sequence substantially homologous thereto, and/or (preferably “and”) said heavy chain variable region comprises a VH CDR1 that has the amino acid sequence of SEQ ID NO:161 or a sequence substantially homologous thereto, a VH CDR2 that has the amino acid sequence of SEQ ID NO:162 or a sequence substantially homologous thereto, and a VH CDR3 that has the amino acid sequence of SEQ ID NO:163 or a sequence substantially homologous thereto; or
(iii) said light chain variable region comprises a VL CDR1 that has the amino acid sequence of SEQ ID NO:110 or a sequence substantially homologous thereto and a VL CDR2 that has the amino acid sequence of SEQ ID NO:111 or a sequence substantially homologous thereto, and/or (preferably “and”) said heavy chain variable region comprises a VH CDR1 that has the amino acid sequence of SEQ ID NO:107 or a sequence substantially homologous thereto, a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto.

In some preferred embodiments, an antibody of the invention comprises at least one heavy chain variable region that comprises three CDRs and at least one light chain variable region that comprises three CDRs, wherein said light chain variable region comprises a VL CDR2 that has the amino acid sequence of SEQ ID NO:57 or a sequence substantially homologous thereto and comprises a VL CDR3 that has the amino acid sequence of SEQ ID NO:58 or a sequence substantially homologous thereto, and wherein said heavy chain variable region comprises a VH CDR1 that has the amino acid sequence of SEQ ID NO:53 or a sequence substantially homologous thereto, and wherein
(i) said light chain variable region comprises a VL CDR1 that has the amino acid sequence of SEQ ID NO:56 or a sequence substantially homologous thereto, and/or (preferably “and”) said heavy chain variable region comprises a VH CDR2 that has the amino acid sequence of SEQ ID NO:54 or a sequence substantially homologous thereto, and a VH CDR3 that has the amino acid sequence of SEQ ID NO:55 or a sequence substantially homologous thereto; or
(ii) said light chain variable region comprises a VL CDR1 that has the amino acid sequence of SEQ ID NO:110 or a sequence substantially homologous thereto, and/or (preferably “and”) said heavy chain variable region comprises a VH CDR2 that has the amino acid sequence of SEQ ID NO:108 or a sequence substantially homologous thereto, and a VH CDR3 that has the amino acid sequence of SEQ ID NO:109 or a sequence substantially homologous thereto.

In some preferred embodiments, an antibody of the invention comprises at least one heavy chain variable region that comprises three CDRs and at least one light chain variable region that comprises three CDRs, wherein said light chain variable region comprises a VL CDR1 that has the amino acid sequence of SEQ ID NO:92 or a sequence substantially homologous thereto, and wherein said heavy chain variable region comprises a VH CDR3 that has the amino acid sequence of SEQ ID NO:91 or a sequence substantially homologous thereto, and wherein
(i) said light chain variable region comprises a VL CDR2 that has the amino acid sequence of SEQ ID NO:93 or a sequence substantially homologous thereto and a VL CDR3 that has the amino acid sequence of SEQ ID NO:94 or a sequence substantially homologous thereto, and/or (preferably “and”) said heavy chain variable region comprises a VH CDR1 that has the amino acid sequence of SEQ ID NO:89 or a sequence substantially homologous thereto and a VH CDR2 that has the amino acid sequence of SEQ ID NO:90 or a sequence substantially homologous thereto; or
(ii) said light chain variable region comprises a VL CDR2 that has the amino acid sequence of SEQ ID NO:147 or a sequence substantially homologous thereto and a VL CDR3 that has the amino acid sequence of SEQ ID NO:148 or a sequence substantially homologous thereto, and/or (preferably “and”) said heavy chain variable region comprises a VH CDR1 that has the amino acid sequence of SEQ ID NO:143 or a sequence substantially homologous thereto and a VH CDR2 that has the amino acid sequence of SEQ ID NO:144 or a sequence substantially homologous thereto.

In some preferred embodiments, an antibody of the invention comprises at least one heavy chain variable region that comprises three CDRs and at least one light chain variable region that comprises three CDRs, wherein said light chain variable region comprises a VL CDR2 that has the amino acid sequence of SEQ ID NO:93 or a sequence substantially homologous thereto, and wherein said heavy chain variable region comprises a VH CDR3 that has the amino acid sequence of SEQ ID NO:91 or a sequence substantially homologous thereto, and wherein
(i) said light chain variable region comprises a VL CDR1 that has the amino acid sequence of SEQ ID NO:92 or a sequence substantially homologous thereto and a VL CDR3 that has the amino acid sequence of SEQ ID NO:94 or a sequence substantially homologous thereto, and/or (preferably “and”) said heavy chain variable region comprises a VH CDR1 that has the amino acid sequence of SEQ ID NO:89 or a sequence substantially homologous thereto and a VH CDR2 that has the amino acid sequence of SEQ ID NO:90 or a sequence substantially homologous thereto; or
(ii) said light chain variable region comprises a VL CDR1 that has the amino acid sequence of SEQ ID NO:20 or a sequence substantially homologous thereto and a VL CDR3 that has the amino acid sequence of SEQ ID NO:22 or a sequence substantially homologous thereto, and/or (preferably “and”) said heavy chain variable region comprises a VH CDR1 that has the amino acid sequence of SEQ ID NO:17 or a sequence substantially homologous thereto and a VH CDR2 that has the amino acid sequence of SEQ ID NO:18 or a sequence substantially homologous thereto.

In some preferred embodiments, an antibody of the invention comprises at least one heavy chain variable region that comprises three CDRs and at least one light chain variable region that comprises three CDRs, wherein said light chain variable region comprises a VL CDR1 that has the amino acid sequence of SEQ ID NO:110 or a sequence substantially homologous thereto and comprises a VL CDR3 that has the amino acid sequence of SEQ ID NO:112 or a sequence substantially homologous thereto, and wherein said heavy chain variable region comprises a VH CDR3 that has the amino acid sequence of SEQ ID NO:109 or a sequence substantially homologous thereto, and wherein
(i) said light chain variable region comprises a VL CDR2 that has the amino acid sequence of SEQ ID NO:111 or a sequence substantially homologous thereto, and/or (preferably “and”) said heavy chain variable region comprises a VH CDR2 that has the amino acid sequence of SEQ ID NO:108 or a sequence substantially homologous thereto, and a VH CDR1 that has the amino acid sequence of SEQ ID NO:107 or a sequence substantially homologous thereto; or
(ii) said light chain variable region comprises a VL CDR2 that has the amino acid sequence of SEQ ID NO:165 or a sequence substantially homologous thereto, and/or (preferably “and”) said heavy chain variable region comprises a VH CDR2 that has the amino acid sequence of SEQ ID NO:162 or a sequence substantially homologous thereto, and a VH CDR1 that has the amino acid sequence of SEQ ID NO:161 or a sequence substantially homologous thereto.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:17, or a sequence substantially homologous thereto,
(b) a VH CDR2 that has the amino acid sequence of SEQ ID NO:18, or a sequence substantially homologous thereto, and
(c) a VH CDR3 that has the amino acid sequence of SEQ ID NO:19, or a sequence substantially homologous thereto; and/or (preferably “and”)

wherein said light chain variable region comprises:
(d) a variable light (VL) CDR1 that has the amino acid sequence of SEQ ID NO:20, or a sequence substantially homologous thereto,
(e) a VL CDR2 that has the amino acid sequence of SEQ ID NO:21, or a sequence substantially homologous thereto, and
(f) a VL CDR3 that has the amino acid sequence of SEQ ID NO:22, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:17, a VH CDR2 of SEQ ID NO:18, and a VH CDR3 of SEQ ID NO:19, and/or (preferably “and”) at least one light chain variable region that comprises a VL CDR1 of SEQ ID NO:20, a VL CDR2 of SEQ ID NO:21, and a VL CDR3 of SEQ ID NO:22.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:35, or a sequence substantially homologous thereto,
(b) a VH CDR2 that has the amino acid sequence of SEQ ID NO:36, or a sequence substantially homologous thereto, and
(c) a VH CDR3 that has the amino acid sequence of SEQ ID NO:37, or a sequence substantially homologous thereto; and/or (preferably “and”)

wherein said light chain variable region comprises:
(d) a variable light (VL) CDR1 that has the amino acid sequence of SEQ ID NO:38, or a sequence substantially homologous thereto,
(e) a VL CDR2 that has the amino acid sequence of SEQ ID NO:39, or a sequence substantially homologous thereto, and
(f) a VL CDR3 that has the amino acid sequence of SEQ ID NO:40, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:35, a VH CDR2 of SEQ ID NO:36, and a VH CDR3 of SEQ ID NO:37, and/or (preferably “and”) at least one light chain variable region that comprises a VL CDR1 of SEQ ID NO:38, a VL CDR2 of SEQ ID NO:39, and a VL CDR3 of SEQ ID NO:40.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:53, or a sequence substantially homologous thereto,
(b) a VH CDR2 that has the amino acid sequence of SEQ ID NO:54, or a sequence substantially homologous thereto, and
(c) a VH CDR3 that has the amino acid sequence of SEQ ID NO:55, or a sequence substantially homologous thereto; and/or (preferably “and”)

wherein said light chain variable region comprises:
(d) a variable light (VL) CDR1 that has the amino acid sequence of SEQ ID NO:56, or a sequence substantially homologous thereto,
(e) a VL CDR2 that has the amino acid sequence of SEQ ID NO:57, or a sequence substantially homologous thereto, and
(f) a VL CDR3 that has the amino acid sequence of SEQ ID NO:58, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:53, a VH CDR2 of SEQ ID NO:54, and a VH CDR3 of SEQ ID NO:55, and/or (preferably “and”) at least one light chain variable region that comprises a VL CDR1 of SEQ ID NO:56, a VL CDR2 of SEQ ID NO:57, and a VL CDR3 of SEQ ID NO:58.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:71, or a sequence substantially homologous thereto,
(b) a VH CDR2 that has the amino acid sequence of SEQ ID NO:72, or a sequence substantially homologous thereto, and
(c) a VH CDR3 that has the amino acid sequence of SEQ ID NO:73, or a sequence substantially homologous thereto; and/or (preferably “and”)

wherein said light chain variable region comprises:
(d) a variable light (VL) CDR1 that has the amino acid sequence of SEQ ID NO:74, or a sequence substantially homologous thereto,
(e) a VL CDR2 that has the amino acid sequence of SEQ ID NO:75, or a sequence substantially homologous thereto, and
(f) a VL CDR3 that has the amino acid sequence of SEQ ID NO:76, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:71, a VH CDR2 of SEQ ID NO:72, and a VH CDR3 of SEQ ID NO:73, and/or (preferably “and”) at least one light chain variable region that comprises a VL CDR1 of SEQ ID NO:74, a VL CDR2 of SEQ ID NO:75, and a VL CDR3 of SEQ ID NO:76.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:89, or a sequence substantially homologous thereto,
(b) a VH CDR2 that has the amino acid sequence of SEQ ID NO:90, or a sequence substantially homologous thereto, and
(c) a VH CDR3 that has the amino acid sequence of SEQ ID NO:91, or a sequence substantially homologous thereto; and/or (preferably “and”)

wherein said light chain variable region comprises:
(d) a variable light (VL) CDR1 that has the amino acid sequence of SEQ ID NO:92, or a sequence substantially homologous thereto,
(e) a VL CDR2 that has the amino acid sequence of SEQ ID NO:93, or a sequence substantially homologous thereto, and
(f) a VL CDR3 that has the amino acid sequence of SEQ ID NO:94, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:89, a VH CDR2 of SEQ ID NO:90, and a VH CDR3 of SEQ ID NO:91, and/or (preferably “and”) at least one light chain variable region that comprises a VL CDR1 of SEQ ID NO:92, a VL CDR2 of SEQ ID NO:93, and a VL CDR3 of SEQ ID NO:94.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:107, or a sequence substantially homologous thereto,
(b) a VH CDR2 that has the amino acid sequence of SEQ ID NO:108, or a sequence substantially homologous thereto, and
(c) a VH CDR3 that has the amino acid sequence of SEQ ID NO:109, or a sequence substantially homologous thereto; and/or (preferably “and”)

wherein said light chain variable region comprises:
(d) a variable light (VL) CDR1 that has the amino acid sequence of SEQ ID NO:110, or a sequence substantially homologous thereto,
(e) a VL CDR2 that has the amino acid sequence of SEQ ID NO:111, or a sequence substantially homologous thereto, and
(f) a VL CDR3 that has the amino acid sequence of SEQ ID NO:112, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:107, a VH CDR2 of SEQ ID NO:108, and a VH CDR3 of SEQ ID NO:109, and/or (preferably “and”) at least one light chain variable region that comprises a VL CDR1 of SEQ ID NO:110, a VL CDR2 of SEQ ID NO:111, and a VL CDR3 of SEQ ID NO:112.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:125, or a sequence substantially homologous thereto,
(b) a VH CDR2 that has the amino acid sequence of SEQ ID NO:126, or a sequence substantially homologous thereto, and
(c) a VH CDR3 that has the amino acid sequence of SEQ ID NO:127, or a sequence substantially homologous thereto; and/or (preferably “and”)

wherein said light chain variable region comprises:
(d) a variable light (VL) CDR1 that has the amino acid sequence of SEQ ID NO:128, or a sequence substantially homologous thereto,
(e) a VL CDR2 that has the amino acid sequence of SEQ ID NO:129, or a sequence substantially homologous thereto, and
(f) a VL CDR3 that has the amino acid sequence of SEQ ID NO:130, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:125, a VH CDR2 of SEQ ID NO:126, and a VH CDR3 of SEQ ID NO:127, and/or (preferably “and”) at least one light chain variable region that comprises a VL CDR1 of SEQ ID NO:128, a VL CDR2 of SEQ ID NO:129, and a VL CDR3 of SEQ ID NO:130.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:143, or a sequence substantially homologous thereto,
(b) a VH CDR2 that has the amino acid sequence of SEQ ID NO:144, or a sequence substantially homologous thereto, and
(c) a VH CDR3 that has the amino acid sequence of SEQ ID NO:145, or a sequence substantially homologous thereto; and/or (preferably “and”)

wherein said light chain variable region comprises:
(d) a variable light (VL) CDR1 that has the amino acid sequence of SEQ ID NO:146, or a sequence substantially homologous thereto,
(e) a VL CDR2 that has the amino acid sequence of SEQ ID NO:147, or a sequence substantially homologous thereto, and
(f) a VL CDR3 that has the amino acid sequence of SEQ ID NO:148, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:143, a VH CDR2 of SEQ ID NO:144, and a VH CDR3 of SEQ ID NO:145, and/or (preferably “and”) at least one light chain variable region that comprises a VL CDR1 of SEQ ID NO:146, a VL CDR2 of SEQ ID NO:147, and a VL CDR3 of SEQ ID NO:148.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:161, or a sequence substantially homologous thereto,
(b) a VH CDR2 that has the amino acid sequence of SEQ ID NO:162, or a sequence substantially homologous thereto, and
(c) a VH CDR3 that has the amino acid sequence of SEQ ID NO:163, or a sequence substantially homologous thereto; and/or (preferably “and”)

wherein said light chain variable region comprises:
(d) a variable light (VL) CDR1 that has the amino acid sequence of SEQ ID NO:164, or a sequence substantially homologous thereto,
(e) a VL CDR2 that has the amino acid sequence of SEQ ID NO:165, or a sequence substantially homologous thereto, and
(f) a VL CDR3 that has the amino acid sequence of SEQ ID NO:166, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:161, a VH CDR2 of SEQ ID NO:162, and a VH CDR3 of SEQ ID NO:163, and/or (preferably “and”) at least one light chain variable region that comprises a VL CDR1 of SEQ ID NO:164, a VL CDR2 of SEQ ID NO:165, and a VL CDR3 of SEQ ID NO:166.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:179, or a sequence substantially homologous thereto,
(b) a VH CDR2 that has the amino acid sequence of SEQ ID NO:180, or a sequence substantially homologous thereto, and
(c) a VH CDR3 that has the amino acid sequence of SEQ ID NO:181, or a sequence substantially homologous thereto; and/or (preferably “and”)

wherein said light chain variable region comprises:
(d) a variable light (VL) CDR1 that has the amino acid sequence of SEQ ID NO:182, or a sequence substantially homologous thereto,
(e) a VL CDR2 that has the amino acid sequence of SEQ ID NO:183, or a sequence substantially homologous thereto, and
(f) a VL CDR3 that has the amino acid sequence of SEQ ID NO:184, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:179, a VH CDR2 of SEQ ID NO:180, and a VH CDR3 of SEQ ID NO:181, and/or (preferably “and”) at least one light chain variable region that comprises a VL CDR1 of SEQ ID NO:182, a VL CDR2 of SEQ ID NO:183, and a VL CDR3 of SEQ ID NO:184.

In some embodiments, the antibody comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:197, or a sequence substantially homologous thereto,
(b) a VH CDR2 that has the amino acid sequence of SEQ ID NO:198, or a sequence substantially homologous thereto, and
(c) a VH CDR3 that has the amino acid sequence of SEQ ID NO:199, or a sequence substantially homologous thereto; and/or (preferably “and”)

wherein said light chain variable region comprises:
(d) a variable light (VL) CDR1 that has the amino acid sequence of SEQ ID NO:200, or a sequence substantially homologous thereto,
(e) a VL CDR2 that has the amino acid sequence of SEQ ID NO:201, or a sequence substantially homologous thereto, and
(f) a VL CDR3 that has the amino acid sequence of SEQ ID NO:202, or a sequence substantially homologous thereto. Substantially homologous sequences are described elsewhere herein. Preferably, said substantially homologous sequence is a sequence containing 1, 2 or 3 amino acid substitutions compared to the given CDR sequence, or said substantially homologous sequence is a sequence containing conservative amino acid substitutions of the given CDR sequence.

In some embodiments, the antibody comprises at least one heavy chain variable region that comprises a VH CDR1 of SEQ ID NO:197, a VH CDR2 of SEQ ID NO:198, and a VH CDR3 of SEQ ID NO:199, and/or (preferably “and”) at least one light chain variable region that comprises a VL CDR1 of SEQ ID NO:200, a VL CDR2 of SEQ ID NO:201, and a VL CDR3 of SEQ ID NO:202.

In one embodiment the invention provides an antibody comprising a VH domain that has the amino acid sequence of SEQ ID NO: 15 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 16 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:16 and/or (preferably “and”) wherein the heavy chain variable region has the amino acid sequence of SEQ ID NO:15.

In one embodiment the invention provides an antibody comprising a VH domain that has the amino acid sequence of SEQ ID NO: 33 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 34 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:34 and/or (preferably “and”) wherein the heavy chain variable region has the amino acid sequence of SEQ ID NO:33.

In one embodiment the invention provides an antibody comprising a VH domain that has the amino acid sequence of SEQ ID NO: 51 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 52 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:52 and/or (preferably “and”) wherein the heavy chain variable region has the amino acid sequence of SEQ ID NO:51.

In one embodiment the invention provides an antibody comprising a VH domain that has the amino acid sequence of SEQ ID NO: 69 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 70 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:70 and/or (preferably “and”) wherein the heavy chain variable region has the amino acid sequence of SEQ ID NO:69.

In one embodiment the invention provides an antibody comprising a VH domain that has the amino acid sequence of SEQ ID NO: 87 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 88 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:88 and/or (preferably “and”) wherein the heavy chain variable region has the amino acid sequence of SEQ ID NO:87.

In one embodiment the invention provides an antibody comprising a VH domain that has the amino acid sequence of SEQ ID NO: 105 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 106 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:106 and/or (preferably “and”) wherein the heavy chain variable region has the amino acid sequence of SEQ ID NO:105.

In one embodiment the invention provides an antibody comprising a VH domain that has the amino acid sequence of SEQ ID NO: 123 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 124 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:124 and/or (preferably “and”) wherein the heavy chain variable region has the amino acid sequence of SEQ ID NO:123.

In one embodiment the invention provides an antibody comprising a VH domain that has the amino acid sequence of SEQ ID NO: 141 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 142 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:142 and/or (preferably “and”) wherein the heavy chain variable region has the amino acid sequence of SEQ ID NO:141.

In one embodiment the invention provides an antibody comprising a VH domain that has the amino acid sequence of SEQ ID NO: 159 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 160 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:160 and/or (preferably “and”) wherein the heavy chain variable region has the amino acid sequence of SEQ ID NO:159.

In one embodiment the invention provides an antibody comprising a VH domain that has the amino acid sequence of SEQ ID NO: 177 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 178 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:178 and/or (preferably “and”) wherein the heavy chain variable region has the amino acid sequence of SEQ ID NO:177.

In one embodiment the invention provides an antibody comprising a VH domain that has the amino acid sequence of SEQ ID NO: 195 or a sequence substantially homologous thereto (e.g. a sequence having at least 80% sequence identity thereto, for example at least 85%, 90%, 95% or 98% sequence identity thereto), and/or (preferably “and”) a VL domain that has the amino acid sequence of SEQ ID NO: 196 or a sequence substantially homologous thereto (e.g. a sequence having at least 80% sequence identity thereto, for example at least 85%, 90%, 95% or 98% sequence identity thereto). In a preferred embodiment, the present invention provides an antibody, wherein the light chain variable region has the amino acid sequence of SEQ ID NO:196 and/or (preferably “and”) wherein the heavy chain variable region has the amino acid sequence of SEQ ID NO:195.

Other preferred embodiments are Ig (e.g. IgG) forms of antibodies described herein, e.g. IgG forms of the 23B9-1, 24C6-1, 27A2-1, 29B5-1, 2B11-1, 33B6-1, 4D11-1, 54F1-1, 69H7-1, 75C9-1 or 9E4-1 antibodies (or antibodies based thereon), preferably full length IgG forms. In some embodiments, the IgG is IgG₁ or IgG₂ (e.g. IgG_{2b}). Thus, in some embodiments the antibody is an Ig antibody comprising CDR sequences and/or a heavy chain variable region and/or a light chain variable region as described herein. It is of course understood that full IgG antibodies will typically comprise two substantially identical heavy chains and two substantially identical light chains.

In some embodiments, antibodies based on the 23B9-1, 24C6-1, 27A2-1, 29B5-1, 2B₁₁-1, 33B6-1, 4D11-1, 54F1-1, 69H7-1, 75C9-1, 9E4-1 antibody sequences set forth in Tables A-K herein are preferred.

Some examples of antibodies of the present invention are the monoclonal antibodies 23B9-1, 24C6-1, 27A2-1, 29B5-1, 2B₁₁-1, 33B6-1, 4D11-1, 54F1-1, 69H7-1, 75C9-1, 9E4-1, sequences of which are shown in Tables A-K herein. These monoclonal antibodies were identified by sequencing B-cell clones from an animal (rabbit) that had been immunized with an OTV16 peptide (SEQ ID NO:3). The CDR domains, VH and VL domains are shown in Tables A-K herein. Antibodies comprising these CDR domains or VH and VL domains (or sequences substantially homologous thereto) are preferred aspects of the invention.

Typically, the monoclonal antibodies 23B9-1, 24C6-1, 27A2-1, 29B5-1, 2B₁₁-1, 33B6-1, 4D11-1, 54F1-1, 69H7-1, 75C9-1 and 9E4-1 (or antibodies based thereon, e.g. antibodies having substantially homologous sequences thereto) bind to (or are capable of binding to, e.g. specifically binding to) an epitope of TRPV1 in the region of TRPV1 defined by amino acid residues 455-470 of TRPV1 (SEQ ID NO:1). In some embodiments, the entire epitope bound lies within this region of TRPV1. In some embodiments, at least one amino acid of the epitope bound lies within this region of TRPV1.

Certain examples of substantially homologous sequences are sequences that have at least 65% identity to the amino acid sequences disclosed. In certain embodiments, the antibodies of the invention comprise at least one light chain variable region that includes an amino acid sequence region of at least about 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO:16, 34, 52, 70, 88, 106, 124, 142, 160, 178 or 196; and/or at least one heavy chain variable region that includes an amino acid sequence region of at least about 65%, 70% or 75%, more preferably at least about 80%, more preferably at least about 85%, more preferably at least about 90% or 95% and most preferably at least about 97%, 98% or 99% amino acid sequence identity to the amino acid sequence of SEQ ID NO:15, 33, 51, 69, 87, 105, 123, 141, 159, 177 or 195.

Other preferred examples of substantially homologous sequences are sequences containing conservative amino acid substitutions of the amino acid sequences disclosed.

Other preferred examples of substantially homologous sequences are sequences containing 1, 2 or 3, preferably 1 or 2 (more preferably 1), altered amino acids in one or more of the CDR regions disclosed. Such alterations might be conserved or non-conserved amino acid substitutions, or a mixture thereof.

In some such embodiments, preferred alterations are conservative amino acid substitutions.

In all embodiments, antibodies containing substantially homologous sequences retain the ability to bind to TRPV1. Preferably, antibodies containing substantially homologous sequences retain one or more (preferably all) of the properties described in relation to the 23B9-1, 24C6-1, 27A2-1, 29B5-1, 2B₁₁-1, 33B6-1, 4D11-1, 54F1-1, 69H7-1, 75C9-1 and/or 9E4-1 antibodies.

Further examples of substantially homologous amino acid sequences in accordance with the present invention are described elsewhere herein.

The CDRs of antibodies of the invention are preferably separated by appropriate framework regions such as those found in naturally occurring antibodies and/or effective engineered antibodies. Thus, the VH, VL and individual CDR sequences of the invention are preferably provided within or incorporated into an appropriate framework or scaffold to enable antigen binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Preferred sequences for framework regions are one or more of the framework regions making up the VH and/or VL domains of the invention, i.e. one or more of the framework regions of the 23B9-1, 24C6-1, 27A2-1, 29B5-1, 2B₁₁-1, 33B6-1, 4D11-1, 54F1-1, 69H7-1, 75C9-1 or 9E4-1 antibodies, as disclosed in Tables A-K herein, or framework regions substantially homologous thereto, and in particular framework regions that allow the maintenance of antigen specificity, for example framework regions that result in substantially the same or the same 3D structure of the antibody.

In certain preferred embodiments, all four of the variable light chain (SEQ ID NOs:27, 28, 29 and 30) and/or variable heavy chain (SEQ ID NOs:23, 24, 25 and 26) framework regions (FR), as appropriate, or FR regions substantially homologous thereto, are found in the antibodies of the invention.

In other preferred embodiments, all four of the variable light chain (SEQ ID NOs:45, 46, 47 and 48) and/or variable heavy chain (SEQ ID NOs:41, 42, 43 and 44) framework regions (FR), as appropriate, or FR regions substantially homologous thereto, are found in the antibodies of the invention.

In other preferred embodiments, all four of the variable light chain (SEQ ID NOs:63, 64, 65 and 66) and/or variable heavy chain (SEQ ID NOs:59, 60, 61 and 62) framework regions (FR), as appropriate, or FR regions substantially homologous thereto, are found in the antibodies of the invention.

In other preferred embodiments, all four of the variable light chain (SEQ ID NOs:81, 82, 83 and 84) and/or variable heavy chain (SEQ ID NOs:77, 78, 79 and 80) framework regions (FR), as appropriate, or FR regions substantially homologous thereto, are found in the antibodies of the invention.

In other preferred embodiments, all four of the variable light chain (SEQ ID NOs:99, 100, 101 and 102) and/or variable heavy chain (SEQ ID NOs:95, 96, 97 and 98) framework regions (FR), as appropriate, or FR regions substantially homologous thereto, are found in the antibodies of the invention.

In other preferred embodiments, all four of the variable light chain (SEQ ID NOs:117, 118, 119 and 120) and/or variable heavy chain (SEQ ID NOs:113, 114, 115 and 116) framework regions (FR), as appropriate, or FR regions substantially homologous thereto, are found in the antibodies of the invention.

In other preferred embodiments, all four of the variable light chain (SEQ ID NOs:135, 136, 137 and 138) and/or variable heavy chain (SEQ ID NOs:131, 132, 133 and 134) framework regions (FR), as appropriate, or FR regions substantially homologous thereto, are found in the antibodies of the invention.

In other preferred embodiments, all four of the variable light chain (SEQ ID NOs:153, 154, 155 and 156) and/or variable heavy chain (SEQ ID NOs:149, 150, 151 and 152) framework regions (FR), as appropriate, or FR regions substantially homologous thereto, are found in the antibodies of the invention.

In other preferred embodiments, all four of the variable light chain (SEQ ID NOs:171, 172, 173 and 174) and/or variable heavy chain (SEQ ID NOs:167, 168, 169 and 170) framework regions (FR), as appropriate, or FR regions substantially homologous thereto, are found in the antibodies of the invention.

In other preferred embodiments, all four of the variable light chain (SEQ ID NOs:189, 190, 191 and 192) and/or variable heavy chain (SEQ ID NOs:185, 186, 187 and 188) framework regions (FR), as appropriate, or FR regions substantially homologous thereto, are found in the antibodies of the invention.

In other preferred embodiments, all four of the variable light chain (SEQ ID NOs:207, 208, 209 and 210) and/or variable heavy chain (SEQ ID NOs:203, 204, 205 and 206) framework regions (FR), as appropriate, or FR regions substantially homologous thereto, are found in the antibodies of the invention.

Antibodies of the present invention may inhibit capsaicin-induced activation of TRPV1.

In some embodiments, inhibition of capsaicin-induced activation of TRPV1 is any measurable or significant inhibition, more preferably a statistically significant inhibition (e.g. as compared to a control with no antibody or as compared to a control with an antibody that does not bind to TRPV1).

In some embodiments, the level of inhibition (or amount of inhibition) of capsaicin-induced activation of TRPV1 observed with (or caused by or elicited by) a control (e.g. a control antibody that does not bind to (or does not specifically bind to) TRPV1) represents (or is set as) the zero inhibition level (or zero inhibition value or 0% inhibition level or value). Thus, in some embodiments, the % inhibitions of capsaicin-induced activation of TRPV1 discussed elsewhere herein are as compared to (or relative to) the inhibition observed with (or caused by or elicited by) a control antibody (e.g. a control antibody that does not bind to TRPV1).

In some embodiments, inhibition of capsaicin-induced activation of TRPV1 is an inhibition of at least 5%, at least 10%, at least 15%, preferably at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100%.

In some embodiments, inhibition of capsaicin-induced activation of TRPV1 is an inhibition of up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95% or up to 100%.

Thus, in some embodiments, inhibition of capsaicin-induced activation of TRPV1 is an inhibition of 5%-100%, 10%-100%, 15%-100%, 20%-100%, 25%-100%, 30%-100%, 35%-100%, 40%-100%, 45%-100%, 50%-100%, 55%-100%, 60%-100%, 65%-100%, 70%-100%, 75%-100%, 80%-100%, 85%-100%, 90%-100% or 95%-100%.

In some embodiments, inhibition of capsaicin-induced activation of TRPV1 is an inhibition of 5%-75%, 10%-75%, 15%-75%, 20%-75%, 25%-75%, 30%-75%, 35%-75%, 40%-75%, 45%-75%, 50%-75%, 55%-75%, 60%-75%, 65%-75% or 70%-75%.

In some embodiments, inhibition of capsaicin-induced activation of TRPV1 is an inhibition of 5%-50%, 10%-50%, 15%-50%, 20%-50%, 25%-50%, 30%-50%, 35%-50%, 40%-50% or 45%-50%.

In some embodiments, inhibition of capsaicin-induced activation of TRPV1 is an inhibition of 5%-25%, 10%-25%, 15%-25% or 20%-25%.

In some preferred embodiments, inhibition of capsaicin-induced activation of TRPV1 is an inhibition of at least 20%, or at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100%.

In some embodiments, antibodies of the invention have an 1050 with respect to inhibition of capsaicin-induced activation of TRPV1 of ≤5 μM, ≤1 μM, ≤900 nM, ≤800 nM, ≤700 nM, ≤600 nM, ≤500 nM, ≤400 nM, ≤300 nM, ≤200 nM, ≤100 nM, ≤75 nM, ≤50 nM, ≤25 nM, ≤10 nM, ≤5 nM, ≤2 nM, nM, ≤500 pM, ≤400 pM, ≤300 pM, ≤200 pM, ≤100 pM, ≤50 pM, ≤25 pM, ≤10 pM, ≤5 pM, ≤2 pM or ≤1 pM. Preferably, the IC50 is ≤1 μM, for example ≤750 nM, ≤500 nM, ≤400 nM, ≤300 nM, ≤200 nM, ≤100 nM, ≤75 nM, ≤50 nM, ≤25 nM, ≤10 nM or ≤5 nM. In some embodiments, the IC50 value may be in the range of 1 pM-≤5 μM, for example 1 pM-1 μM, 1 pM-500 nM, 1 pM-100 nM, 1 pM-50 nM, 500 pM-10 μM, 500 pM-1 μM, 500 pM-500 nM, 500 pM-100 nM, 500 pM-50 nM, 1 nM-10 μM, 1 nM-1 μM, 1 nM-500 nM, 1 nM-100 nM, 1 nM-50 nM, 10 nM-10 μM, 10 nM-1 μM, 10 nM-500 nM, 10 nM-100 nM, 10 nM-50 nM, 100 nM-10 μM, 100 nM-1 μM, or 100 nM-500 nM. In some embodiments, the IC50 value may be up to 5 μM, or up to 1 μM, or up to 900 nM, or up to 800 nM, or up to 700 nM, or up to 600 nM, or up to 500 nM, or up to 400 nM, or up to 300 nM, or up to 200 nM, or up to 100 nM, or up to 50 nM or up to 10 nM. An IC50 value represents the half maximal inhibitory concentration of a substance for a biological process under study, in the context of the present invention the half maximal inhibitory concentration of an antibody for the inhibition of capsaicin-induced activation of TRPV1. IC50 values in the context of the present invention may be alternatively viewed as half maximal inhibitory concentration of an antibody for the inhibition of capsaicin-induced cellular TRPV1-mediated Ca²⁺ influx. IC50 values may be calculated by any suitable means (and be based on any suitable tests, methods or assays, for example methods as described herein). For example, IC50 values may be established (or calculated) based on the results of a FLIPR method.

As indicated above, in some embodiments, antibodies may preferentially inhibit capsaicin-induced activation of TRPV1 as opposed to heat-induced activation of TRPV1.

In some embodiments, the inhibition of heat-induced activation of TRPV1 (if any) by an antibody of the invention is substantially the same as (or not significantly different from or analogous to or comparable to) the inhibition of heat-induced activation of TRPV1 observed with (or caused by or elicited by) a control (e.g. a control antibody that does not bind to (or does not specifically bind to) TRPV1). In some embodiments, the level of inhibition (or amount of inhibition) of heat-induced activation of TRPV1 observed with (or caused by or elicited by) a control (e.g. a control antibody that does not bind to (or does not specifically bind to) TRPV1) represents (or is set as) the zero inhibition level (or zero inhibition value or 0% inhibition level or value). Thus, in some embodiments, the % inhibitions of heat-induced activation of TRPV1 discussed elsewhere herein are as compared to (or relative to) the inhibition observed with (or caused by or elicited by) a control antibody (e.g. a control antibody that does not bind to TRPV1).

In some embodiments, antibodies of the present invention inhibit heat-induced activation of TRPV1 by no more than 25%, preferably by no more than 20%, or by no more than 15%, preferably by no more than 10%, by no more than 5%, by no more than 4%, by no more than 3%, by no more than 2%, or by no more than 1% or by 0%.

Thus, in some embodiments, antibodies of the present invention inhibit heat-induced activation of TRPV1 by 0%-25%, 0%-20%, 0%-10%, 0%-5%, 0%-4%, 0%-3%, 0%-2%, 0%-1% or 0%.

In some preferred embodiments, antibodies of the present invention cause (or elicit) no measurable inhibition of heat-induced activation of TRPV1 or no significant inhibition (preferably no statistically significant inhibition) of heat-induced activation of TRPV1.

In some embodiments, the above inhibitions are as determined when the antibody is used at a concentration in the micromolar (μM), nanomolar (nM) or picomolar (pM) range, preferably the nanomolar (nM) or picomolar (pM) range. Thus, in some embodiments, the above inhibitions are as determined when the antibody is used at a concentration of ≤10 μM, ≤5 μM, ≤1 μM, ≤900 nM, ≤800 nM, ≤700 nM, ≤600 nM, ≤500 nM, ≤400 nM, ≤300 nM, ≤200 nM, ≤100 nM, ≤75 nM, ≤50 nM, ≤25 nM, ≤10 nM, ≤5 nM, ≤2 nM, ≤1 nM, ≤500 pM, ≤400 pM, ≤300 pM, ≤200 pM, ≤100 pM, ≤50 pM, ≤25 pM, ≤10 pM, ≤5 pM, ≤2 pM or ≤1 pM. Thus, in some embodiments, the above inhibitions are as determined when the antibody is used at a concentration of 1 pM-≤10 μM, for example 1 pM-1 μM, 1 pM-500 nM, 1 pM-100 nM, 1 pM-50 nM, 500 pM-10 μM, 500 pM-1 μM, 500 pM-500 nM, 500 pM-100 nM, 500 pM-50 nM, 1 nM-10 μM, 1 nM-1 μM, 1 nM-500 nM, 1 nM-100 nM, 1 nM-50 nM, 10 nM-10 μM, 10 nM-1 μM, 10 nM-500 nM, 10 nM-100 nM, 10 nM-50 nM, 100 nM-10 μM, 100 nM-1 μM, or 100 nM-500 nM. In some embodiments, the above inhibitions are as determined when the antibody is used at a concentration of up to 5 μM, or up to 1 μM, or up to 900 nM, or up to 800 nM, or up to 700 nM, or up to 600 nM, or up to 400 nM, or up to 300 nM, or up to 200 nM, or up to 100 nM, or up to 50 nM or up to 10 nM.

In some embodiments, the above inhibitions (e.g. % inhibitions) and concentrations apply when the antibody is a polyclonal antibody (e.g. a rabbit polyclonal antibody). In some embodiments, the above inhibitions (e.g. % inhibitions) and concentrations apply when the antibody is a monoclonal antibody.

As indicated above, in some embodiments, antibodies may preferentially inhibit capsaicin-induced activation of TRPV1 as opposed to heat-induced activation of TRPV1. In such embodiments, this means that the given antibody inhibits (or is capable of inhibiting) capsaicin-induced activation of TRPV1 to a greater extent than it inhibits (or is capable of inhibiting) heat-induced activation of TRPV1. Thus, in such embodiments, if a given antibody inhibits capsaicin-induced activation of TRPV1 by X %, that antibody will inhibit heat-induced activation of TRPV1 by <X %.

In some embodiments, the % inhibition (or % inhibition value) of capsaicin-induced activation of TRPV1 is at least 5% higher, but typically at least 10% higher, preferably at least 20% higher, at least 30% higher, at least 40% higher, at least 50% higher, at least 60% higher, at least 70% higher, at least 80% higher, at least 90% or even 100% higher than the % inhibition (or % inhibition value) of heat-induced activation of TRPV1.

In some embodiments, the % inhibition (or % inhibition value) of capsaicin-induced activation of TRPV1 is at least 20% and the % inhibition (or % inhibition value) of heat-induced activation of TRPV1 is ≤10% or is ≤5% or is 0%. In some embodiments, the % inhibition (or % inhibition value) of capsaicin-induced activation of TRPV1 is at least 40% and the % inhibition (or % inhibition value) of heat-induced activation of TRPV1 is ≤10% or is ≤5% or is 0%. In some embodiments, the % inhibition (or % inhibition value) of capsaicin-induced activation of TRPV1 is at least 60% and the % inhibition (or % inhibition value) of heat-induced activation of TRPV1 is ≤10% or is ≤5% or is 0%. In some embodiments, the % inhibition (or % inhibition value) of capsaicin-induced activation of TRPV1 is at least 80% and the % inhibition (or % inhibition value) of heat-induced activation of TRPV1 is ≤10% or is ≤5% or is 0%.

In some embodiments, antibodies preferentially inhibit capsaicin-induced activation of TRPV1 as opposed to heat-induced activation of TRPV1 as determined by performing a test (or assay) to determine (or quantify) the level of (or amount of) inhibition of capsaicin-induced activation of TRPV1 and a test (or assay to) to determine the level of (or amount of) inhibition of heat-induced activation of TRPV1. Suitable assays are described elsewhere herein.

In some embodiments, antibodies preferentially inhibit (or are capable of inhibiting) capsaicin-induced activation of TRPV1 as opposed to heat-induced activation of TRPV1 as determined when the antibody is used at the same concentration in both such assays (i.e. the same concentration of antibody is used in the test to determine the level of inhibition of capsaicin-induced activation of TRPV1 as is used in the test to determine the level of inhibition of heat-induced activation of TRPV1).

In some embodiments, antibodies preferentially inhibit capsaicin-induced activation of TRPV1 as opposed to heat-induced activation of TRPV1 as determined when the antibody is used at at least a 1.5 times. 2 times, at least a 3 times, at least a 4 times, preferably at least a 5 times, (e.g. a 1.5, 2, 3, 4, 5, 6, 7, 8, 9 or 10 times or a 2-5, 3-5, or 4-5 times) higher concentration in the test to determine the level of inhibition of heat-induced activation of TRPV1 concentration than is used in test the to determine the level of inhibition of capsaicin-induced activation of TRPV1.

In some embodiments, for antibodies that bind to an isolated peptide of SEQ ID NO:2 or SEQ ID NO:3 (OTV16 peptides) (or isolated peptides substantially homologous thereto) or that bind to an epitope of TRPV1 that corresponds to (or corresponds essentially to) such an isolated peptide, the inhibition of capsaicin-induced activation of TRPV1 is an inhibition of at least 10%, at least 15%, at least 20%, at least 25%, at least 30%, at least 35%, at least 40% or at least 45% or at least 50%.

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, the inhibition of capsaicin-induced activation of TRPV1 is an inhibition of up to 10%, up to 15%, up to 20%, up to 25%, up to 30%, up to 35%, up to 40%, up to 45% or up to 50%.

In some embodiments, the above-mentioned % inhibitions of capsaicin-induced activation of TRPV1 for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide are as determined when said antibody (e.g. a polyclonal antibody such as a rabbit polyclonal antibody) is used at a concentration of 500 pM to 1 μM, e.g. 1 nM to 100 nM, or about 20 nM (e.g. 26.7 nM).

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, inhibition of heat-induced activation of TRPV1 is by no more than 5%, by no more than 4%, by no more than 3%, by no more than 2% or by no more than 1%, preferably 0% preferably there is no measurable (or no significant) inhibition of heat-induced activation of TRPV1.

Thus, in some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, the inhibition of heat-induced activation of TRPV1 is 0%-5%, 0%-4%, 0%-3%, 0%-2%, 0%-1% or preferably 0%.

In some embodiments, the above-mentioned % inhibitions of heat-induced activation of TRPV1 for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide are as determined when said antibody (e.g. a polyclonal antibody such as a rabbit polyclonal antibody) is used at a concentration of 500 pM to 10 μM, e.g. 1 nM to 500 nM, or about 10 nM (e.g. 13.4 nM) or about 100 nM (e.g. 134 nM).

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, no more than 5%, no more than 4%, no more than 3%, no more than 2% or no more than 1%, preferably 0%, preferably no measurable (or no significant) inhibition of heat-induced activation of TRPV1 is observed when the antibody is used at about a 5 times higher concentration than an antibody concentration that inhibits capsaicin-induced activation of TRPV1 by more than 5%, at least 10%, at least 15%, at least 20%, at least 25%, at least 30%, at least 35%, at least 40% or at least 45% or at least 50%.

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, no more than 5%, no more than 4%, no more than 3%, no more than 2% or no more than 1%, preferably no measurable (or no significant) inhibition of heat-induced activation of TRPV1 is observed when said antibody is used at a concentration of about 100 nM (e.g. 134 nM) and at least 20%, at least 25%, at least 30%, at least 35%, at least 40% or at least 45% or at least 50% inhibition of capsaicin-induced activation of TRPV1 is observed when said antibody is used at a concentration of about 20 nM (e.g. 26.7 nM).

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, no more than 5% inhibition of heat-induced activation of TRPV1 is observed when said antibody is used at a concentration of about 100 nM (e.g. 134 nM) and at least 20% inhibition of capsaicin-induced activation of TRPV1 is observed when said antibody is used at a concentration of about 20 nM (e.g. 26.7 nM).

In some embodiments, the above-mentioned inhibitions (e.g. % inhibitions) of capsaicin-induced activation of TRPV1 are as determined in a patch-clamp method, e.g. as described elsewhere herein.

In some embodiments, the above-mentioned inhibitions (e.g. % inhibitions) of heat-induced activation of TRPV1 are as determined as described elsewhere herein.

In some embodiments, for antibodies that bind to an isolated peptide of SEQ ID NO:2 or SEQ ID NO:3 (OTV16 peptides) (or isolated peptides substantially homologous thereto) or that bind to an epitope of TRPV1 that corresponds to (or corresponds essentially to) such an isolated peptide, the inhibition of capsaicin-induced activation of TRPV1 is an inhibition of at least 5%, at least 10%, at least 15%, at least 20% or at least 25%, at least 30% or at least 35%, at least 40% or at least 45% or at least 50%. In some embodiments, these % inhibitions (e.g. % inhibitions) of capsaicin-induced activation of TRPV1 are as determined by a calcium imaging method, e.g. as described elsewhere herein.

In some embodiments, for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide, the inhibition of capsaicin-induced activation of TRPV1 is an inhibition of up to 5%, up to 10%, up to 15%, up to 20%, up to 25%, up to 30%, up to 40%, up to 50% or up to 60%. In some embodiments, these % inhibitions (e.g. % inhibitions) of capsaicin-induced activation of TRPV1 are as determined by a calcium imaging method, e.g. as described elsewhere herein.

In some embodiments, the above-mentioned % inhibitions of capsaicin-induced activation of TRPV1 for antibodies that bind to an isolated peptide of SEQ ID NO:2 or SEQ ID NO:3 (or isolated peptides substantially homologous thereto) or that bind to an epitope of TRPV1 that corresponds to (or corresponds essentially to) such an isolated peptide are as determined when said antibody (e.g. a polyclonal antibody such as a rabbit polyclonal antibody) is used at a concentration of 1 nM to 100 nM (e.g. 2.6 nM). In some embodiments, these % inhibitions (e.g. % inhibitions) of capsaicin-induced activation of TRPV1 are as determined by a calcium imaging method, e.g. as described elsewhere herein.

In some embodiments, capsaicin-induced activation of TRPV1 is the TRPV1 activation induced when capsaicin is present (or used at or contacted with TRPV1) at a concentration of 10 nM to 10 μM, for example 100 nM to 1 μM (e.g. a concentration of 100 nM or 1 μM). In some embodiments, capsaicin-induced activation of TRPV1 is the TRPV1 activation induced when capsaicin is present at 100 nM. In some embodiments, capsaicin-induced activation of TRPV1 is the TRPV1 activation induced when capsaicin is present at 300 nM.

Capsaicin-induced activation of TRPV1 and inhibition of capsaicin-induced activation of TRPV1 may be assessed by any appropriate method and the skilled person will be familiar with suitable methods.

In some embodiments, capsaicin-induced activation of TRPV1 and inhibition of capsaicin-induced activation of TRPV1 may be assessed (or be as assessed) using an electrophysiological method, such as a patch-clamp technique. Patch-clamp methods are well-known in the art and also described herein. In accordance with the present invention a reduction (or inhibition or lowering) of capsaicin-induced currents by an antibody of the invention as compared to a control antibody (e.g. a control antibody that does not bind to (or does not specifically bind to) TRPV1) is typically indicative that the antibody inhibits capsaicin-induced activation of TRPV1.

In some embodiments, the patch-clamp method comprises steps of clamping (e.g. with a pipette) a TRPV1 expressing cell (e.g. in a bath) and recording the current (or current signal). Typically, the current (or current signal) is measured (e.g. in parallel tests) in cells stimulated (or incubated) with capsaicin and in cells stimulated with capsaicin and an antibody of the invention and also preferably in cells stimulated (or incubated) with capsaicin and a control antibody (e.g. a control antibody that does not bind to (or does not specifically bind to) TRPV1). A reduction (or inhibition or lowering) of capsaicin-induced currents, e.g. as compared to a control antibody, is typically indicative that the antibody of the invention inhibits capsaicin-induced activation of TRPV1.

In some embodiments, in a patch clamp method whole cell recordings are performed using a microfluidic device for patch clamp recordings together with a patch clamp amplifier. In some embodiments, the cells are Chinese hamster ovary (CHO) cells expressing TRPV1. In some embodiments, bath and pipette solutions contain buffer F and G (as defined elsewhere herein), respectively. In some embodiments, the cells are clamped (e.g. at −60 mV) and the current signals are recorded with a sampling frequency (e.g. of 10 kHz) and low pass filtered (e.g. at 2 kHz). In some embodiments, the patch-clamp recordings are acquired using digital/analogue sampling and acquisition software. In preferred embodiments, current amplitudes are measured by exposing cells to capsaicin, with and without an antibody of the invention or a control antibody. In preferred embodiments, the cells are exposed to 100 nM capsaicin in buffer F for ˜20 s, followed by buffer F for 60 s, antibody in buffer F for 60 s and then 100 nM capsaicin together with antibody in buffer F for ˜20 s. In preferred embodiments, measurements where the seal resistance shifted largely during treatment were excluded from analysis. In preferred embodiments, the recorded amplitude of the peak during stimulation with antibody+capsaicin is divided by the recorded amplitude of the peak during stimulation with capsaicin. The obtained value may be multiplied by 100 to obtain the cell response during antibody+capsaicin stimulation as a percentage of the control response (capsaicin, i.e. capsaicin only). Thus, in some embodiments, % inhibition of capsaicin-induced stimulation (activation) of TRPV1 may be calculated as (1−(recorded amplitude of the peak during stimulation with antibody+capsaicin divided by the recorded amplitude of the peak during stimulation with capsaicin only)×100). Measurements are preferably performed on cells from at least two different cell culture dishes. A particularly preferred patch-clamp method is described in the Example section herein.

In some embodiments, a patch clamp method comprises stimulating (or activating) TRPV1 expressing cells four times using capsaicin (e.g. 100 nM capsaicin or 300 nM capsaicin). In some such embodiments, hTRPV1 expressing cells are pre-treated with an antibody of the invention prior to the third activation (or stimulation) and antibody is included together with capsaicin during the third activation (or stimulation). In some such embodiments, the first, second and fourth activations (stimulations) are with capsaicin only. The amplitude of the third current peak in the presence of antibody may be compared to the mean of the amplitudes of current peaks two and four. In some embodiments, cells are treated twice with capsaicin alone to obtain peaks 1 and 2, and then are treated with antibody and then antibody together with capsaicin to obtain peak 3, followed by capsaicin alone to obtain peak 4. In some embodiments, the % inhibition may be calculated as (1−((peak 3)/((peak2+peak4)/2)))*100. In some other embodiments, cells are first treated twice with capsaicin alone to obtain peaks 1 and 2, then treated with antibody or vehicle and then antibody or vehicle together with capsaicin to obtain peak 3, followed by capsaicin alone to obtain peak 4. In some such embodiments, the % inhibition may be calculated as (1−((peak3_{Ab}/peak2_{Ab})/(peak3ᵥₑₕ/peak2ᵥₑₕ)))*100.

In some embodiments, capsaicin-induced activation of TRPV1 and inhibition of capsaicin-induced activation of TRPV1 may be assessed (or be as assessed) using a calcium imaging method. Capsaicin induces calcium uptake.

In some embodiments, capsaicin-induced activation of TRPV1 and inhibition of capsaicin-induced activation of TRPV1 may be assessed (or be as assessed) by (i) loading TRPV1 expressing cells with a calcium indicator (e.g. as described elsewhere herein), (ii) contacting (or incubating) said cells with an antibody of the invention (or a control antibody or a vehicle only (i.e. no antibody) control), and (iii) contacting said cells (i.e. the cells that have been incubated with an antibody of the invention or control in (ii)) with (or exposing said cells to) capsaicin and calcium (Ca²⁺), and (iv) assessing (or determining or measuring) the ability of an antibody of the invention to reduce (or lower or inhibit) capsaicin-induced calcium influx (or uptake) to the cells by measuring (or determining) a signal (e.g. a fluorescence signal) from the calcium indicator (e.g. as compared to a control antibody or a vehicle only control). Preferred features of such embodiments will be evident from the discussion elsewhere herein and these may be applied mutatis mutandis to the embodiments discussed in this paragraph. In some embodiments, if the signal from the calcium indicator measured is lower in cells that were contacted with an antibody of the invention prior to the contacting with capsaicin and calcium (Ca²⁺) as compared to the signal measured in cells that were contacted with a control antibody or vehicle (vehicle only) prior to the contacting with capsaicin and calcium (Ca²⁺), then that is typically indicative that the antibody of the invention inhibits capsaicin-induced activation of TRPV1. In some embodiments, when more than one concentration of a given antibody of the invention is tested (e.g. if serial dilutions of an antibody are tested), if the signal from the calcium indicator measured (or determined) is lower (or reduced) as the concentration of the antibody (i.e. antibody of the invention) is increased (i.e. lower signal at a higher concentration of the antibody as compared to at a lower concentration of the antibody, e.g. lower signal at higher (or increased) concentrations in an antibody dilution series), then that is typically indicative that the antibody of the invention inhibits capsaicin-induced activation of TRPV1.

In some calcium imaging methods for assessing capsaicin-induced activation of TRPV1 and inhibition of capsaicin-induced activation of TRPV1 a calcium indicator (e.g. Fluo-3 AM) is used. In some such methods cells (e.g. CHO cells) expressing hTRPV1 are incubated with a calcium indicator to “load” the cells with the calcium indicator (e.g. Fluo-3 AM e.g. 4.4 μM thereof) for e.g. 30 min at e.g. 37° C. and preferably then washed (the calcium indicator remains inside the cells after washing) and then incubated with an antibody of the invention or with a control antibody (e.g. dissolved in PBS) or with no additional agent for e.g. 1 h, at e.g. room temperature. The control antibody is typically an antibody that does not bind to (or does not specifically bind to) TRPV1. Typically, the cells are then contacted with capsaicin (e.g. 1 μM) and Ca²⁺ (e.g. 150 μM) (e.g. the capsaicin and calcium is added to the antibody solution covering the cells) and the calcium content within the cells is monitored (or measured) by measuring the fluorescence intensity (of the calcium indicator), e.g. with a plate reader. Typically, the fluorescence intensity observed after (or during) incubation with capsaicin and calcium where the cells were prior incubated with an antibody of the invention is normalised with respect to the fluorescence intensity observed after (or during) incubation with capsaicin and calcium (Ca²⁺) where the cells were not prior incubated with an antibody of the invention or with any additional agent. If the fluorescence intensity is reduced (or inhibited or lowered) by the incubation with an antibody of the invention prior to the incubation with capsaicin and calcium (Ca²⁺) as compared to when there is no incubation with an antibody of the invention prior to the incubation with capsaicin and calcium (Ca²⁺) (e.g. as compared to when there is an incubation with a control antibody prior to the incubation with capsaicin and calcium (Ca²⁺)), then that is typically indicative that the antibody of the invention inhibits capsaicin-induced activation of TRPV1. A particularly preferred calcium imaging method is described in the Example section herein.

In some embodiments, capsaicin-induced activation of TRPV1 and inhibition of capsaicin-induced activation of TRPV1 may be assessed (or be as assessed) using a Fluorescence Imaging Plate Reader (FLIPR) method.

In certain preferred FLIPR methods, TRPV1 expressing cells (e.g. TRPV1 expressing CHO cells) are cultured in black, clear bottom, microplates (e.g. 96-well microplates). Cells are then loaded with a calcium indicator (e.g. Fluo-3 AM, e.g. 4 μM thereof) by incubating the cells with a calcium indicator (e.g. 4 μM Fluo-3 AM) in a buffer, e.g. HEPES buffer (140 nM NaCl, 5 mM KCl, 1 mM CaCl₂), 1 mM MgCl2, 10 mM HEPES, 10 mM D-glucose, pH 7.4) for e.g. 30 minutes at e.g. room temperature. The cells are then washed with a buffer (e.g. HEPES) to remove extracellular calcium indicator (e.g. Fluo-3 AM) (the calcium indicator remains inside the cells after washing). Subsequently, an antibody of the invention that is diluted into a buffer (e.g. a calcium containing buffer such as HEPES) is added to wells, for example 100 μl of the antibody containing buffer is added to the cells in each well (typically serial dilutions of the antibody diluted into buffer (e.g. a calcium containing buffer, such as HEPES) are added to wells, i.e. different concentrations of antibody in different wells). Cells are incubated in the presence of antibody (e.g. for 4 minutes at e.g. room temperature). Fluorescence measurements are typically made by a microplate reader e.g. with excitation at 483 nm (bandwidth 14 nm) and emission at 530 (bandwidth 30 nm). Baseline fluorescence intensity is first measured, then (i.e. subsequently) a fixed amount of capsaicin in a calcium containing buffer (e.g. in 100 μl of buffer) such as HEPES is added to each well (to give a concentration of capsaicin that had been previously established as representing the EC50 value of capsaicin for the batch of cells under study, typically such a concentration is in the sub-micromolar range, for example such a concentration may be in the lower nM range, e.g. 10 nM) and a second fluorescence intensity measurement is performed after a set time (typically within minutes, e.g. 1 to 5 or 1 to 10 or 1 to 20 minutes). The fluorescence intensity (of the calcium indicator) provides a report (or read-out) of the amount of calcium within the cells (and thus on the capsaicin-induced activation of TRPV1). This FLIPR assay relies on there being calcium (Ca 2+) present in the buffer when capsaicin is added (i.e. relies on calcium being present in the buffer when the cells are exposed to capsaicin) and any suitable calcium containing buffer may be used (the skilled person will be familiar with suitable buffers), for example the above-mentioned HEPES buffer provides sufficient calcium). The antibody of the invention may be in PBS (phosphate buffered saline) prior to dilution (i.e. prior to being diluted) into a buffer (e.g. a calcium containing buffer such as HEPES) for adding to the cells (wells). EC50 is the concentration of a substance that gives half-maximal response of a biological process, in this case TRPV1-mediated Ca²⁺ entry (or TRPV1-mediated Ca²⁺ uptake) into cells. Data may be presented (or obtained or determined) as the Fluorescence rate, which is calculated as fluorescence at a set (or certain) time after addition of capsaicin minus the (baseline) fluorescence measured before capsaicin addition. If a reduction in the fluorescence rate is observed (or measured or determined) as the concentration of the antibody (i.e. of a given antibody being tested) is increased (e.g. at higher (or increased) concentrations in an antibody dilution series), then that is typically indicative that the antibody of the invention inhibits capsaicin-induced activation of TRPV1. The 1050 values for the tested antibodies may be calculated (and the skilled person can readily do this). The 1050 value is the half maximal inhibitory concentration of a substance for a biological process under study, in this case capsaicin induced cellular TRPV1-mediated Ca²⁺ influx (or uptake or entry).

In certain other preferred FLIPR methods, TRPV1 expressing cells (e.g. hTRPV1 expressing CHO cells) are cultured in black, clear bottom, microplates (e.g. 96-well microplates). Preferably, the wells of the plates are coated in poly-D-Lysine. Cells are then loaded with a calcium indicator (e.g. Calcium 6) by incubating the cells with a calcium indicator (e.g. Calcium 6) in a buffer (e.g. 100 μl/well), e.g. HEPES-buffered salt solution (140 mM NaCl, 5 mM KCl, 1 mM CaCl₂), 1 mM MgCl₂, 10 mM HEPES, 10 mM Glucose, pH 7.4), e.g. for 60 minutes (at e.g. 37° C. at e.g. 5% CO₂). The cells may then be equilibrated (or allowed to come to) to ambient temperature (or room temperature) in the dark, for e.g. 15 minutes. The calcium indicator solution (i.e. the calcium indicator in buffer) is then removed and, subsequently, an antibody of the invention that is diluted into a buffer (e.g. a calcium containing buffer such as HEPES-buffered salt solution) is added to wells, for example 100 μl of the antibody containing buffer is added to the cells in each well (typically serial dilutions of the antibody diluted into buffer (e.g. a calcium containing buffer, such as HEPES-buffered salt solution) are added to wells, i.e. different concentrations of antibody in different wells). An initial measurement of fluorescence intensity is then taken by a microplate reader (e.g. λₑₓ=485 nm, λₑₘ=525 nm; 4 cycles; 12 seconds per cycle; 10 flashes per cycle). Cells are incubated in the presence of antibody (e.g. for 30 minutes at e.g. 25° C.), typically this incubation is done with the microplate inside microplate reader. Then (i.e. subsequently) a fixed amount of capsaicin in a calcium containing buffer (e.g. in 100 μl of buffer) such as HEPES-buffered salt solution is added to each well (typically to give a concentration of capsaicin that had been previously established as representing the EC50 value of capsaicin for the batch of cells under study). After the addition of capsaicin, the change in fluorescence intensity is followed for a given period of time, e.g. for 5 minutes, by taking a series of fluorescent intensity measurements over time (in the microplate reader, e.g. with the microplate reader in kinetic reading mode), e.g. λₑₓ=485 nm, λₑₘ=525 nm; 25 cycles; 12 seconds per cycle; 10 flashes per cycle. The fluorescence intensity measurements (of the calcium indicator) provides a report (or read-out) of the amount of calcium within the cells (and thus on the capsaicin-induced activation of TRPV1). This FLIPR assay relies on there being calcium (Ca²⁺) present in the buffer when capsaicin is added (i.e. relies on calcium being present in the buffer when the cells are exposed to capsaicin) and any suitable calcium containing buffer may be used (the skilled person will be familiar with suitable buffers), for example the above-mentioned HEPES-buffered salt solution provides sufficient calcium). The antibody of the invention may be in PBS (phosphate buffered saline) prior to dilution (i.e. prior to being diluted) into a buffer (e.g. a calcium containing buffer such as HEPES-buffered salt solution) for adding to the cells (wells). EC50 is the concentration of a substance that gives half-maximal response of a biological process, in this case TRPV1-mediated Ca²⁺ entry (or TRPV1-mediated Ca²⁺ uptake) into cells. If the fluorescence (or fluorescence measurement(s)) determined (at a given time point after capsaicin addition or over a given time period after capsaicin addition) is lower (or reduced) after cells have been incubated (pre-incubated) with an antibody of the invention as compared to after cells have (instead) been incubated (pre-incubated) with vehicle only or with a control antibody that does not bind to TRPV1, then that is typically indicative that the antibody of the invention inhibits capsaicin-induced activation of TRPV1. When more than one concentration of a given antibody of the invention is tested (e.g. if serial dilutions of an antibody are tested), if the fluorescence (or fluorescence measurement(s)) determined (at a given time point after capsaicin addition or over a given time period after capsaicin addition) is lower (or reduced) as the concentration of the antibody (i.e. of a given antibody being tested) is increased (i.e. lower at a higher concentration of the antibody as compared to at a lower concentration of the antibody, e.g. lower at higher (or increased) concentrations in an antibody dilution series), then that is typically indicative that the antibody of the invention inhibits capsaicin-induced activation of TRPV1. In some embodiments, a given time point after capsaicin addition may be 12, 24, 36, 48, 60, 72, 84, 96, 108, 120, 132, 144, 156, 168, 180, 192, 204, 216, 228, 240, 252, 264, 276, 288 or 300 seconds). In some embodiments, a given time period after capsaicin addition is 5 minutes (300 seconds). Fluorescence (or a fluorescence measurement) may be expressed as a fluorescence ratio. A fluorescence ratio may be determined at a series of time points (e.g. at 12 second intervals, such as at 12, 24, 36, 48, 60, 72, 84, 96, 108, 120, 132, 144, 156, 168, 180, 192, 204, 216, 228, 240, 252, 264, 276, 288 and 300 seconds), e.g. over a 5 minute period, after capsaicin addition. A fluorescence ratio may be calculated as ΔF(t)/F₀ (where ΔF(t)=Fₜ−F₀; Fₜ is the fluorescence intensity after capsaicin EC₅₀ addition at the time t, and F₀ is the initial fluorescence intensity measured immediately after antibody loading). t may=0 to 300 seconds. In some embodiments, a ratio may be calculated at a plurality (e.g. 5, 10, 15, 20, 25 or 26) of time points (a plurality of “t”s over a given period of time (e.g. over 5 minutes) after capsaicin addition. In some such embodiments, a ratio may be calculated at a time point “t” every 12 seconds for 5 minutes, e.g. “for t=0 seconds, for t=12 seconds, for t=24 seconds, etc. until t=300 seconds). A kinetic curve may be plotted for a given concentration of antibody tested (or for each concentration of antibody tested in the case where more than one concentration of antibody is tested, e.g. if a dilution series of antibody concentrations is tested) based on calculated (or measured) fluorescence ratios (i.e. curves based on the ratios calculated at a series of time points after capsaicin addition (e.g. time points after capsaicin addition as discussed above) or over a time period after capsaicin addition (e.g. a time period after capsaicin addition as discussed above). The value of the area under the curve (AUC) (i.e. area under the kinetic curve) may be determined (or calculated) for a given concentration of antibody. If more than one antibody concentration is tested (e.g. if a dilution series of antibody concentrations is tested) dose response curves may be built (or plotted). When AUCs (or AUC values) are determined for more than one concentration of a given antibody, if the AUC value is lower (or reduced) as the concentration of the antibody (i.e. of a given antibody being tested) is increased (i.e. lower at a higher concentration of the antibody as compared to at a lower concentration of the antibody, e.g. lower at higher (or increased) concentrations in an antibody dilution series), then that is typically indicative that the antibody of the invention inhibits capsaicin-induced activation of TRPV1. In some embodiments, when an antibody of the invention is used at a concentration of about 5 μM, an AUC value for (or obtained for or calculated for) an antibody of the invention that is at least 10%, preferably at least 20%, at least 30%, at least 40% or at least 50% lower than an AUC value for a control (e.g. for a vehicle only control or for a control antibody that does not bind to TRPV1) is typically indicative that the antibody inhibits capsaicin-induced activation of TRPV1. In some embodiments, when an antibody of the invention is used at a concentration of about 10 μM, an AUC value for (or obtained for or calculated for) an antibody of the invention that is at least 10%, preferably at least 20%, at least 30%, at least 40% or at least 50% lower than an AUC value (control AUC value) for a control (e.g. for a vehicle only control or for a control antibody that does not bind to TRPV1) is typically indicative that the antibody inhibits capsaicin-induced activation of TRPV1. The 1050 values for the tested antibodies may be calculated (and the skilled person can readily do this). The 1050 value is the half maximal inhibitory concentration of a substance for a biological process under study, in this case capsaicin induced cellular TRPV1-mediated Ca²⁺ influx (or uptake or entry).

Exemplary and preferred FLIPR methods are described in Example 1 and Example 2 herein.

As indicated above, certain methods comprise the use of a calcium indicator (e.g. Fluo3-AM or Calcium 6). The skilled person is familiar with such indicators. Calcium indicators may be conveniently used to visualise whether or not (or the extent to which) the intracellular concentration (or amount) of calcium ions (Ca²⁺) is increased during (or after) exposure to a particular stimulus (e.g. capsaicin or heat). Such calcium indicators can be loaded into cells (prior to the exposure to a stimulus) and their signal (e.g. fluorescence) increases upon binding to Ca²⁺ ions (i.e. upon influx of Ca²⁺ ions into the cell, e.g. from a Ca²⁺ ion containing medium or buffer).

Heat-induced activation of TRPV1 is typically the TRPV1 activation induced at (or as determined at) 42° C. Thus, in some embodiments, heat-induced activation of TRPV1 is the TRPV1 activation induced at 42° C.-45° C., 42° C.-50° C., 42° C.-60° C., 42° C.-70° C., 42° C.-80° C., 42° C.-90° C. or 42° C.-100° C. In a preferred embodiment, heat-induced activation of TRPV1 is the TRPV1 activation induced at (or as determined at) 42° C.-45° C. In a preferred embodiment, heat-induced activation of TRPV1 is the TRPV1 activation induced at (or as determined at) 42° C. In another preferred embodiment, heat-induced activation of TRPV1 is the TRPV1 activation induced at (or as determined at) 45° C.

Heat-induced activation of TRPV1 (and inhibition of heat-induced activation of TRPV1) may be assessed by any appropriate method and the skilled person will be familiar with suitable methods. Heat (e.g. 42° C., preferably 42° C.) induces the opening of the TRPV1 ion channel and leads to an influx of calcium ions (Ca²⁺) into TRPV1 expressing cells. Thus, heat-induced activation of TRPV1 (and inhibition of heat-induced activation of TRPV1) may be assessed by determining whether or not (or the extent to which) the intracellular concentration (or amount) of calcium ions (Ca²⁺) is increased during (or after) heating. A calcium indicator (e.g. Fluo-3 or Fluo3-AM or Calcium 6) may be conveniently used to visualise whether or not (or the extent to which) the intracellular concentration (or amount) of calcium ions (Ca²⁺) is increased during (or after) heating. Such calcium indicators can be loaded into cells (prior to the exposure to heat) and their fluorescence increases upon binding to Ca²⁺ ions (i.e. upon influx of Ca²⁺ ions into the cell, e.g. from a Ca²⁺ ion containing medium). This can be visualised by any convenient means e.g. by confocal microscopy. Whether or not (or the extent to which) a TRPV1 binding antibody is capable of inhibiting heat-induced activation of TRPV1 may be assessed by contacting the TRPV1 expressing cells with said antibody (e.g. by delivering the antibody to the cells using a microfluidic device, e.g. a Biopen as described in the Example section herein) and determining whether or not (or the extent to which) heat-induced activation of TRPV1 is inhibited (or reduced) as compared to the heat-induced activation of TRPV1 in the absence of the antibody (e.g. as compared to the heat-induced activation of TRPV1 observed in the presence of a control antibody that does not bind to (or that does not specifically bind to) TRPV1). The heat (e.g. 42° C.) may be provided using any suitable means, e.g. a laser heating system, e.g. as described in the Example section herein. A reduction (e.g. a significant reduction) in heat-induced activation of TRPV1 in the presence of an anti-TRPV1 antibody indicates that the antibody inhibits heat-induced activation of TRPV1. The absence of a reduction (or no significant reduction) in heat-induced activation of TRPV1 in the presence of an anti-TRPV1 antibody indicates that the antibody does not (or does not significantly) inhibit heat-induced activation of TRPV1. A particularly preferred method of assessing heat-induced activation of TRPV1 (and inhibition of heat-induced activation of TRPV1) is described in the Example section herein.

In some embodiments, heat-induced activation of TRPV1 (and inhibition thereof) may be assessed (or be as assessed) using a method in which TRPV1 expressing cells receive two pulses of heat. In some such embodiments, the cells are first pulsed with heat, followed by a cool-down, followed by administration of (or contacting with) antibody and a heat pulse in combination, followed by a cool-down. The effect of an antibody on heat-induced activation of TRPV1 may be assessed by determining the effect of the antibody on heat-induced influx of calcium ions into the cells. The influx of calcium into the cells (peak amplitude) may be measured using a calcium indicator (e.g. Fluo-3). The ratio of the second (second heat pulse or peak 2) to first (first heat pulse or peak 1) peak amplitude may be calculated for both antibody and vehicle. In some embodiments, the percent of inhibition may be calculated by comparing the ratio for the antibody to the ratio for the vehicle. Thus, in some embodiments, % inhibition may be (1−((Peak2_{Ab}/Peak1_{Ab})/(peak2ᵥₑₕ/peak1ᵥₑₕ)))*100.

In some embodiments, antibodies of the present invention may inhibit (or be capable of inhibiting) NADA-induced activation of TRPV1. NADA (N-arachidonoyl dopamine) is a potent natural TRPV1 agonist.

In some embodiments, inhibition of NADA-induced activation of TRPV1 by an antibody of the invention is any measurable or significant inhibition (e.g. as compared to a control with no antibody or as compared to a control with an antibody that does not bind to TRPV1). In some embodiments, the amounts (or levels) of inhibition of NADA-induced activation of TRPV1 (e.g. % inhibitions) are as described elsewhere herein in relation to the inhibition of capsaicin-induced activation of TRPV1.

In some embodiments, NADA-induced activation of TRPV1 is the TRPV1 activation induced when NADA is present (or used at or contacted with TRPV1) at a concentration of 10 nM to 10 μM, for example 100 nM to 1 μM (e.g. a concentration of 100 nM or 1 μM). In some embodiments, NADA-induced activation of TRPV1 is the TRPV1 activation induced when NADA is present at 1 μM.

In some embodiments, the NADA-induced activation of TRPV1 (or inhibition thereof) is as determined in a patch-clamp method, e.g. as described elsewhere herein, with NADA being used instead of capsaicin.

As indicated above, preferably antibodies of the invention bind to human TRPV1 (SEQ ID NO:1). In some embodiments of the present invention, antibodies of the invention may additionally bind to (or be capable of binding to) a non-human primate TRPV1 and/or additionally bind to mouse (murine; Mus musculus) TRPV1 and/or additionally bind to rat (Rattus norvegicus) TRPV1. In some such embodiments, the antibody is the 23B9-1, 24C6-1, 27A2-1, 29B5-1, 2B₁₁-1, 3366-1, 4D11-1, 54F1-1, 69H7-1, 75C9-1 or 9E4-1 antibody of the invention, or an antibody based thereon (e.g. an antibody having CDR sequences, or VH domain and/or VL domain sequences that are substantially homologous sequences thereto).

Thus, in some preferred embodiments, antibodies of the invention may bind to human TRPV1 and bind to a non-human primate TRPV1 (preferably Macaca mulatta TRPV1). In some such embodiments, the antibody is the 23B9-1, 24C6-1, 27A2-1, 29B5-1, 2B₁₁-1, 33B6-1, 4D11-1, 54F1-1, 69H7-1, 75C9-1 or 9E4-1 antibody of the invention, or an antibody based thereon.

In some embodiments, antibodies of the invention may bind to human TRPV1 and bind to mouse TRPV1. In some such embodiments, the antibody is the 23B9-1, 2B₁₁-1, 54F1-1 or 9E4-1 antibody of the invention, or an antibody based thereon.

In some embodiments, antibodies of the invention may bind to human TRPV1 and bind to rat TRPV1. In some such embodiments, the antibody is the 2B₁₁-1 or 54F1-1 antibody of the invention, or an antibody based thereon.

In some embodiments, antibodies of the invention may bind to human TRPV1, bind to a non-human primate (NHP) TRPV1 (preferably Macaca mulatta TRPV1) and bind to rat TRPV1. In some such embodiments, the antibody is the 2B₁₁-1 or 54F1-1 antibody of the invention, or an antibody based thereon.

In some embodiments, antibodies of the invention may bind to human TRPV1, to a non-human primate TRPV1 (preferably Macaca mulatta TRPV1) and to mouse TRPV1. In some such embodiments, the antibody is the 23B9-1, 2B₁₁-1, 54F1-1 or 9E4-1 antibody of the invention, or an antibody based thereon.

In some embodiments, antibodies of the invention may bind to human TRPV1, bind to a non-human primate TRPV1 (preferably Macaca mulatta TRPV1), bind to rat TRPV1 and bind to mouse TRPV1. In some such embodiments, the antibody is the 2B₁₁-1 or 54F1-1 antibody of the invention, or an antibody based thereon.

The amino acid sequence of human TRPV1 is set out herein as SEQ ID NO:1. The amino acid sequence of a non-human primate (specifically the non-human primate Macaca mulatta) TRPV1 is set out herein as SEQ ID NO:10. Macaca mulatta TRPV1 is a preferred non-human primate TRPV1 in accordance with the invention. Other non-human primate TRPV1s may include Macaca fascicularis TRPV1 and Macaca nemestrina TRPV1. The amino acid sequence of mouse TRPV1 is set out herein as SEQ ID NO:11. The amino acid sequence of rat TRPV1 is set out herein as SEQ ID NO:12.

Such cross-reactivity between species and in particular between humans and species commonly used as pre-clinical animal models (e.g. non-human primates, mice or rats)) may be an advantage as it may allow a more effective translation from pre-clinical studies to clinical use. For example, having an antibody which cross reacts with the native TRPV1 present in a non-human animal model used may mean that the results in this model are more likely to reflect the situation in a human patient, thereby allowing a more accurate assessment of for example dosing to be made and an increased likelihood of identifying any potentially relevant or problematic side effects. For example, the ability of an antibody of the invention to bind to both human TRPV1 and a relevant non-human animal TRPV1 (e.g. a non-human primate TRPV1 or mouse TRPV1 or rat TRPV1) means that such antibodies may be tested in preclinical toxicity studies in such non-human animals to assess adverse side effects of the treatment and to find appropriate tolerated dosages. The ability of an antibody to bind to TRPV1 (e.g. human TRPV1 and/or a non-human primate TRPV1 and/or mouse TRPV1 and/or rat TRPV1) may be assessed by any suitable method, for example an SPR (surface plasmon resonance) assay or an ELISA assay.

As indicated above, preferably antibodies of the invention bind to an isolated peptide comprising (or consisting of) the amino acid sequence of SEQ ID NO:2 or SEQ ID NO:3 (preferably SEQ ID NO:3). The isolated peptide of SEQ ID NO:2 corresponds to residues 455-470 of human TRPV1 (SEQ ID NO:1), and the isolated peptide of SEQ ID NO:3 corresponds essentially to residues 455-470 of human TRPV1 (SEQ ID NO:1). In some embodiments of the present invention, antibodies of the invention may additionally bind to (or be capable of binding to) an isolated peptide comprising (or consisting of) the amino acid sequence of SEQ ID NO:4 or SEQ ID NO:5 (preferably SEQ ID NO:5), and/or additionally bind to an isolated peptide comprising (or consisting of) the amino acid sequence of SEQ ID NO:6 or SEQ ID NO:7 (preferably SEQ ID NO:7) and/or additionally bind to an isolated peptide comprising (or consisting of) the amino acid sequence of SEQ ID NO:8 or SEQ ID NO:9 (preferably SEQ ID NO:9). In some such embodiments, the antibody is the 23B9-1, 24C6-1, 27A2-1, 29B5-1, 2B₁₁-1, 33B6-1, 4D11-1, 54F1-1, 69H7-1, 75C9-1 or 9E4-1 antibody of the invention, or an antibody based thereon (e.g. an antibody having CDR sequences, or VH domain and/or VL domain sequences that are substantially homologous sequences thereto).

Thus, in some preferred embodiments, antibodies of the invention may bind to an isolated peptide comprising (or consisting of) the amino acid sequence of SEQ ID NO:2 or SEQ ID NO:3 and bind to an isolated peptide comprising (or consisting of) the amino acid sequence of SEQ ID NO:4 or SEQ ID NO:5. In some such embodiments, the antibody is the 23B9-1, 24C6-1, 27A2-1, 29B5-1, 2B₁₁-1, 3366-1, 4D11-1, 54F1-1, 69H7-1, 75C9-1 or 9E4-1 antibody of the invention, or an antibody based thereon.

In some embodiments, antibodies of the invention may bind to an isolated peptide comprising (or consisting of) the amino acid sequence of SEQ ID NO:2 or SEQ ID NO:3 and bind to an isolated peptide comprising (or consisting of) the amino acid sequence of SEQ ID NO:8 or SEQ ID NO:9. In some such embodiments, the antibody is the 23B9-1, 2B₁₁-1, 54F1-1 or 9E4-1 antibody of the invention, or an antibody based thereon.

In some embodiments, antibodies of the invention may bind to an isolated peptide comprising (or consisting of) the amino acid sequence of SEQ ID NO:2 or SEQ ID NO:3 and bind to an isolated peptide comprising (or consisting of) the amino acid sequence of SEQ ID NO:6 or SEQ ID NO:7. In some such embodiments, the antibody is the 2B₁₁-1 or 54F1-1 antibody of the invention, or an antibody based thereon.

In some embodiments, antibodies of the invention may bind to an isolated peptide comprising (or consisting of) the amino acid sequence of SEQ ID NO:2 or SEQ ID NO:3, bind to an isolated peptide comprising (or consisting of) the amino acid sequence of SEQ ID NO:4 or SEQ ID NO:5 and bind to an isolated peptide comprising (or consisting of) the amino acid sequence of SEQ ID NO:6 or SEQ ID NO:7. In some such embodiments, the antibody is the 2B₁₁-1 or 54F1-1 antibody of the invention, or an antibody based thereon.

In some embodiments, antibodies of the invention may bind to an isolated peptide comprising (or consisting of) the amino acid sequence of SEQ ID NO:2 or SEQ ID NO:3, bind to an isolated peptide comprising (or consisting of) the amino acid sequence of SEQ ID NO:4 or SEQ ID NO:5 and bind to an isolated peptide comprising (or consisting of) the amino acid sequence of SEQ ID NO:8 or SEQ ID NO:9. In some such embodiments, the antibody is the 23B9-1, 2B₁₁-1, 54F1-1 or 9E4-1 antibody of the invention, or an antibody based thereon.

In some embodiments, antibodies of the invention may bind to an isolated peptide comprising (or consisting of) the amino acid sequence of SEQ ID NO:2 or SEQ ID NO:3, bind to an isolated peptide comprising (or consisting of) the amino acid sequence of SEQ ID NO:4 or SEQ ID NO:5, bind to an isolated peptide comprising (or consisting of) the amino acid sequence of SEQ ID NO:6 or SEQ ID NO:7 and bind to an isolated peptide comprising (or consisting of) the amino acid sequence of SEQ ID NO:8 or SEQ ID NO:9. In some such embodiments, the antibody is the 2B₁₁-1 or 54F1-1 antibody of the invention, or an antibody based thereon.

The isolated peptides of SEQ ID NO:4 and SEQ ID NO:5 comprise an amino acid sequence (SEQ ID NO:4) that corresponds to the region of Macaca mulatta TRPV1 that is defined by residues 455-470 of Macaca mulatta TRPV1 (SEQ ID NO:10). The isolated peptides of SEQ ID NO:4 and SEQ ID NO:5 comprise an amino acid sequence (SEQ ID NO:4) that is also found in Macaca fascicularis TRPV1 and Macaca nemestrina TRPV1. The isolated peptides of SEQ ID NO:8 and SEQ ID NO:9 comprise an amino acid sequence (SEQ ID NO:8) that corresponds to the region of mouse TRPV1 that is defined by residues 455-471 of mouse TRPV1 (SEQ ID NO:11). The isolated peptides of SEQ ID NO:6 and SEQ ID NO:7 comprise an amino acid sequence (SEQ ID NO:6) that corresponds essentially to the region of rat TRPV1 that is defined by residues 454-470 of rat TRPV1 (SEQ ID NO:12). These regions of Macaca mulatta, mouse and rat TRPV1 may be considered analogous to the region of human TRPV1 that is defined by the residues 455-470 of human TRPV1 (SEQ ID NO:1).

The ability to bind to an isolated peptide of SEQ ID NO:4 or SEQ ID NO:5 is indicative that an antibody binds to (or is capable of binding to) a non-human primate TRPV1 (preferably Macaca mulatta TRPV1). The ability to bind to an isolated peptide of SEQ ID NO:6 or SEQ ID NO:7 is indicative that an antibody binds to (or is capable of binding to) rat TRPV1. The ability to bind to an isolated peptide of SEQ ID NO:8 or SEQ ID NO:9 is indicative that an antibody binds to (or is capable of binding to) mouse TRPV1.

The ability of an antibody to bind to an isolated peptide may be assessed by any suitable method, for example an SPR (surface plasmon resonance) assay, or an ELISA assay. Typically, in such an ELISA assay the isolated peptide is the bait peptide (or target peptide).

In some embodiments, the ability to bind to an isolated peptide (e.g. SEQ ID NO:2, 3, 4, 5, 6, 7, 8, and/or 9) may be determined by (or is as determined by) an ELISA assay comprising:
(a) coating wells of an ELISA plate with a relevant isolated peptide (e.g. a peptide of SEQ ID NO:2, 3, 4, 5, 6, 7, 8 or 9), for example by applying the peptide (e.g. 1 μg/ml, e.g. in PBS) to wells of the ELISA plate and incubating (e.g. for 1 h at e.g. 37° C.);
(b) washing said wells of said ELISA plate (e.g. with PBS containing 0.05% Tween);
(c) adding blocking buffer (e.g. PBS, 0.5% BSA, 0.05% Tween 20, pH 7.4) to said wells and incubating (e.g. for 1 h at e.g. 37° C.);
(d) adding an antibody of the invention (e.g. a dilution series of an antibody of the invention), e.g. in blocking buffer, to said wells and incubating (e.g. for 1 h at e.g. ambient temperature (or room temperature));
(e) washing said wells of said ELISA plate (e.g. with PBS containing 0.05% Tween);
(f) adding a secondary antibody that is conjugated to a detectable moiety or label (e.g. alkaline phosphatase), e.g. in blocking buffer, to said wells and incubating (e.g. for 1 h at e.g. ambient temperature (or room temperature));
(g) washing said wells of said ELISA plate (e.g. with PBS containing 0.05% Tween);
(h) detecting signal produced by the detectable moiety (or label) that is conjugated to the secondary antibody. If signal is detected (e.g. increased (or higher) signal as compared to a negative control) this is indicative that the antibody is able to bind to the peptide. If the detectable moiety/label is an enzyme, then a substrate of the enzyme may be added in order to detect signal. For example, if the detectable moiety is alkaline phosphatase then pNPP (p-nitrophenyl phosphate), e.g. 1 mg/ml pNPP, may be added (e.g. in pNPP buffer), and absorbance (signal) may be measured at 405 nm.

In ELISA assays, binding may be as compared to a negative control Appropriate negative controls would be well-known to a person skilled in the art. For example, a negative control may be a well (or wells) that is not coated with an isolated peptide under investigation. For example, a negative control may be a well (or wells) coated with bovine serum albumin (BSA) protein. Thus, a negative control may be the binding (or level of binding) observed (or determined) in wells that are not coated with an isolated peptide under investigation. A negative control may be the binding (or level of binding) observed or determined in wells that are coated with bovine serum albumin (BSA) protein.

In another aspect, the present invention provides an antibody, for example an isolated antibody, which binds to (or specifically recognises or specifically binds to) TRPV1, wherein said antibody is capable of inhibiting capsaicin-induced activation of TRPV1 to a greater extent than heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds) TRPV1, wherein said antibody selectively inhibits capsaicin-induced activation of TRPV1 as opposed to heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds to) TRPV1, wherein said antibody preferentially inhibits capsaicin-induced activation of TRPV1 as compared to heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds to) TRPV1, wherein said antibody preferentially inhibits capsaicin-induced activation of TRPV1 over heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds to) TRPV1, wherein said antibody inhibits capsaicin-induced activation of TRPV1 without significantly inhibiting heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to TRPV1 (or specifically recognises or specifically binds to), wherein said antibody does not significantly inhibit heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody (e.g. a monoclonal antibody), which binds to (or specifically recognises or specifically binds) TRPV1, wherein said antibody inhibits capsaicin-induced activation of TRPV1. Discussion of various features of the antibodies of other aspects of the invention and preferred embodiments may be applied mutatis mutandis to this aspect of the invention.

In another aspect, the present invention provides an antibody, for example an isolated antibody, which binds to (or specifically recognises or specifically binds to) TRPV1, wherein said antibody binds to an isolated epitope or conjugate of the invention. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds) TRPV1, wherein said antibody preferentially inhibits capsaicin-induced activation of TRPV1 as opposed to heat-induced activation of TRPV1. Discussion of various features of the antibodies of other aspects of the invention and preferred embodiments apply mutatis mutandis to this aspect of the invention.

In another aspect, the present invention provides an antibody, for example an isolated antibody, which binds to (or specifically recognises or specifically binds) TRPV1 in the region of TRPV1 consisting of amino acid residues 455-470 of TRPV1 (SEQ ID NO:1). Discussion of various features of the antibodies of other aspects of the invention and preferred embodiments may be applied mutatis mutandis to this aspect of the invention.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”. The operable limits and parameters of combinations, as with the amounts of any single agent, will be known to those of ordinary skill in the art in light of the present disclosure.

In addition, where the terms “comprise”, “comprises”, “has” or “having”, or other equivalent terms are used herein, then in some more specific embodiments these terms include the term “consists of” or “consists essentially of”, or other equivalent terms.

Nucleic acid molecules comprising nucleotide sequences that encode the antibodies of the present invention as defined herein or parts or fragments thereof, form yet further aspects of the invention.

Other preferred nucleic acid molecules are those encoding a VH region of an antibody of the present invention (e.g. those encoding SEQ ID NOs:15 or 33 or 51 or 69 or 87 or 105 or 123 or 141 or 159 or 177 or 195 or sequences substantially homologous thereto, such as SEQ ID NOs:13 or 31 or 49 or 67 or 85 or 103 or 121 or 139 or 157 or 175 or 193 or sequences substantially homologous thereto). Other preferred nucleic acid molecules are those encoding a VL region of an antibody of the present invention (e.g. those encoding SEQ ID NOs:16 or 34 or 52 or 70 or 88 or 106 or 124 or 142 or 160 or 178 or 196 or sequences substantially homologous thereto, such as SEQ ID NOs:14 or 32 or 50 or 68 or 86 or 104 or 122 or 140 or 158 or 176 or 194, respectively, or sequences substantially homologous thereto).

Thus, certain preferred nucleic acid molecules comprise a sequence which encodes a heavy chain variable region (VH) that has the amino acid sequence of SEQ ID NO: 15 or 33 or 51 or 69 or 87 or 105 or 123 or 141 or 159 or 177 or 195 or a sequence substantially homologous thereto (which are preferably encoded by SEQ ID NO: 13 or 31 or 49 or 67 or 85 or 103 or 121 or 139 or 157 or 175 or 193 or a sequence substantially homologous thereto) and/or comprise a sequence which encodes a light chain variable region (VL) which has the amino acid sequence of SEQ ID NO: 16 or 34 or 52 or 70 or 88 or 106 or 124 or 142 or 160 or 178 or 196 or a sequence substantially homologous thereto (which are preferably encoded by SEQ ID NO: 14 or 32 or 50 or 68 or 86 or 104 or 122 or 140 or 158 or 176 or 194 or a sequence substantially homologous thereto).

Also preferred are nucleic acids which encode the following combinations: SEQ ID NOs: 15 and 16; or SEQ ID NOs: 33 and 34; or SEQ ID NOs 51 and 52; or SEQ ID NOs 69 and 70; or SEQ ID NOs 87 and 88; or SEQ ID NOs 105 and 106; or SEQ ID NOs 123 and 124; or SEQ ID NOs 141 and 142; or SEQ ID NOs 159 and 160; or SEQ ID NOs 177 and 178; or SEQ ID NOs 195 and 196 (or sequences substantially homologous thereto).

Also preferred are nucleic acid molecules which comprise the following combinations: SEQ ID NOs: 13 and 14; or SEQ ID NOs: 31 and 32; or SEQ ID NOs 49 and 50; or SEQ ID NOs 67 and 68; or SEQ ID NOs 85 and 86; or SEQ ID NOs 103 and 104; or SEQ ID NOs 121 and 122; or SEQ ID NOs 139 and 140; or SEQ ID NOs 157 and 158; or SEQ ID NOs 175 and 176; or SEQ ID NOs 193 and 194 (or sequences substantially homologous thereto).

Other preferred nucleic acid molecules comprise sequences that encode IgG forms of the antibodies of the invention.

In another aspect, the present invention provides a set (or plurality) of nucleic acid molecules each comprising a nucleotide sequence, wherein said set of nucleic acid molecules together (or collectively) encode an antibody in accordance with the invention. Such a set of nucleic acid molecules may be characterised in that when the set is expressed (i.e. expressed together) (e.g. in a host cell) an entire antibody of the present invention is expressed and preferably assembled.

The term “substantially homologous” as used herein in connection with an amino acid or nucleic acid sequence includes sequences having at least 65%, 70% or 75%, preferably at least 80%, and even more preferably at least 85%, 90%, 95%, 96%, 97%, 98% or 99%, sequence identity to the amino acid or nucleic acid sequence disclosed. Substantially homologous sequences of the invention thus include single or multiple base or amino acid alterations (additions, substitutions, insertions or deletions) to the sequences of the invention. At the amino acid level preferred substantially homologous sequences contain up to 5, e.g. only 1, 2, 3, 4 or 5, preferably 1, 2 or 3, more preferably 1 or 2, altered amino acids, in one or more of the framework regions and/or one or more of the CDRs making up the sequences of the invention. Said alterations can be with conservative or non-conservative amino acids. Preferably said alterations are conservative amino acid substitutions. Conservative amino acid substitutions discussed elsewherein.

Other preferred examples of substantially homologous sequences are sequences having at least 50%, at least 55%, at least 60%, at least 65%, at least 70%, at least 75%, at least 80%, at least 85%, at least 90% or at least 95% sequence identity to one or more of the CDR sequences disclosed. Thus, in some embodiments, a “substantially homologous” CDR sequence may be a sequence having at least 50%, at least 55%, at least 60%, at least 65%, at least 70%, at least 75%, at least 80%, at least 85%, at least 90% or at least 95% sequence identity to a given CDR sequence described herein. Altered residues might be conserved or non-conserved amino acid substitutions, or a mixture thereof. In some embodiments, preferred alterations are conservative amino acid substitutions.

In some embodiments, in antibodies having a “substantially homologous” sequence as compared to a given sequence, or having a certain degree of sequence identity as compared to a given sequence, the altered amino acid residues(s) are not in a CDR region. For example, in some embodiments, in antibodies having a VH domain sequence and/or a VL domain sequence that has a certain degree of sequence identity to a given VH domain and/or a given VL domain sequence of a particular antibody of the invention (e.g. 23B9-1, 24C6-1, 27A2-1, 29B5-1, 2B₁₁-1, 33B6-1, 4D11-1, 54F1-1, 69H7-1, 75C9-1 or 9E4-1), the altered (or variant) residue(s) are not in a CDR region. Thus, in some embodiments, in antibodies having a “substantially homologous” sequence as compared to a given sequence, or having a certain degree of sequence identity as compared to a given sequence, the altered amino acid residues(s) are in one or more framework regions.

As is evident from elsewhere herein, in other embodiments, in antibodies having a “substantially homologous” sequence as compared to a given sequence, or having a certain degree of sequence identity as compared to a given sequence, the altered amino acid residues(s) may be in a CDR region.

In certain embodiments, if a given starting sequence is relatively short (e.g. four amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR3 sequence in accordance with the present invention, e.g. a starting VH CDR3 sequence which in some embodiments may be four amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 50%, at least 75%, 80%, 85%, 90%, 95%, 96%, 97%, 98% or 99%.

In some embodiments, in an antibody having a “substantially homologous” sequence as compared to a given sequence, or having a certain degree of sequence identity as compared to a given sequence, the three VL CDR amino acid sequences and the three VH CDR amino acid sequences (i.e. all six CDR sequences taken together) are considered together to be the whole (or entire) CDR complement of the antibody, and the amino acid sequence of said whole CDR complement of said antibody is at least 70%, preferably at least 80%, or at least 90%, or at least 95% identical to the corresponding whole (or entire) CDR complement of a given starting (or reference) antibody. The starting (or reference) antibody may have the CDR sequences of the 23B9-1, 24C6-1, 27A2-1, 29B5-1, 2B₁₁-1, 33B6-1, 4D11-1, 54F1-1, 69H7-1, 75C9-1 or 9E4-1 antibodies of the present invention.

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antibody should retain the ability to bind to TRPV1 as described above. Preferably, any substantially homologous antibody should retain one or more (or all) of the functional capabilities of the starting antibody.

Substantially homologous sequences of antibodies of the invention also include, without limitation, for example alterations that do not affect the VH, VL or CDR domains of the antibodies, e.g. antibodies where tag sequences or other components are added that do not contribute to the binding of antigen, or alterations to convert one type or format of antibody molecule or fragment to another type or format of antibody molecule or fragment (e.g. conversion from Fab to scFv or whole antibody or vice versa), or the conversion of an antibody molecule to a particular class or subclass of antibody molecule (e.g. the conversion of an antibody molecule to IgG or a subclass thereof, e.g. IgG₂ or IgG₄).

Preferably, any substantially homologous antibody should retain the ability to specifically bind to the same (or substantially the same) epitope of TRPV1 as recognized by the antibody in question, for example, the same epitope recognized by the CDR domains of the invention or the VH and VL domains of the invention as described herein. Thus, preferably, any substantially homologous antibody should retain the ability to compete with one or more of the various antibodies of the invention (e.g. one or more of the described polyclonal antibodies or one or more of the described monoclonal antibodies 23B9-1, 24C6-1, 27A2-1, 29B5-1, 2B₁₁-1, 33B6-1, 4D11-1, 54F1-1, 69H7-1, 75C9-1 or 9E4-1) for binding to TRPV1. Binding to the same epitope/antigen can be readily tested by methods well known and described in the art, e.g. using binding assays, e.g. a competition assay. Retention of other functional properties can also readily be tested by methods well known and described in the art or herein.

Thus, a person skilled in the art will appreciate that binding assays can be used to test whether “substantially homologous” antibodies have the same binding specificities as the antibodies and antibody fragments of the invention, for example, binding assays such as competition assays or ELISA assays as described elsewhere herein. BIAcore assays could also readily be used to establish whether “substantially homologous” antibodies can bind to TRPV1. The skilled person will be aware of other suitable methods and variations.

As outlined below, a competition binding assay can be used to test whether “substantially homologous” antibodies retain the ability to specifically bind to substantially the same epitope (or the same epitope) of TRPV1 as recognized by the antibodies of the invention (e.g. antibodies 23B9-1, 24C6-1, 27A2-1, 29B5-1, 2B₁₁-1, 33B6-1, 4D11-1, 54F1-1, 69H7-1, 75C9-1 or 9E4-1, or antibodies based on these antibodies), or have the ability to compete with one or more of the various antibodies of the invention (e.g. antibodies 23B9-1, 24C6-1, 27A2-1, 29B5-1, 2B11-1, 33B6-1, 4D11-1, 54F1-1, 69H7-1, 75C9-1 or 9E4-1, or antibodies based on these antibodies). The method described below is only one example of a suitable competition assay. The skilled person will be aware of other suitable methods and variations.

An exemplary competition assay involves assessing the binding of various effective concentrations of an antibody of the invention (e.g. a rabbit polyclonal antibody that binds to an isolated peptide or epitope of TRPV1 of the invention, or e.g. an antibody selected from 23B9-1, 24C6-1, 27A2-1, 29B5-1, 2B₁₁-1, 33B6-1, 4D11-1, 54F1-1, 69H7-1, 75C9-1 or 9E4-1, or an antibody based thereon) to TRPV1 in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to TRPV1) is evidence of binding to substantially the same epitope. Preferably, the test antibody significantly reduces the amount of antibody of the invention that binds to TRPV1. Preferably, the test antibody reduces the amount of antibody of the invention that binds to TRPV1 by at least about 60%, 65%, 70%, 75%, 80%, 85%, 90% or 95%. ELISA and flow cytometry assays may be used for assessing inhibition of binding in such a competition assay but other suitable techniques would be well known to a person skilled in the art.

In some embodiments, “substantially homologous” antibodies which retain the ability to specifically bind to substantially the same (or the same) epitope of TRPV1 as recognized by the antibodies of the invention (e.g. antibodies 23B9-1, 24C6-1, 27A2-1, 29B5-1, 2B₁₁-1, 33B6-1, 4D11-1, 54F1-1, 69H7-1, 75C9-1 or 9E4-1, or antibodies based on these antibodies) or which have the ability to compete with one or more of the various antibodies of the invention (e.g. antibodies 23B9-1, 24C6-1, 27A2-1, 29B5-1, 2B₁₁-1, 33B6-1, 4D11-1, 54F1-1, 69H7-1, 75C9-1 or 9E4-1, or antibodies based on these antibodies) are preferred.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to TRPV1. Preferably, the competing antibody can bind to the same epitope as the reference antibody. Alternatively viewed, the competing antibody preferably has the same epitope specificity as the reference antibody.

“Reference antibodies” as used herein include antibodies (e.g. the polyclonal rabbit antibodies described herein) that bind to an isolated peptide of the invention or to an epitope of TRPV1 in accordance with the invention. “Reference antibodies” also include antibodies which can bind to TRPV1 in accordance with the invention which preferably have a VH and a VL domain as defined herein, more preferably a VH domain of SEQ ID NO: 15 and a VL domain of SEQ ID NO: 16, or a VH domain of SEQ ID NO: 33 and a VL domain of SEQ ID NO: 34, or a VH domain of SEQ ID NO: 51 and a VL domain of SEQ ID NO: 52, or a VH domain of SEQ ID NO: 69 and a VL domain of SEQ ID NO: 70, or a VH domain of SEQ ID NO: 87 and a VL domain of SEQ ID NO: 88, or a VH domain of SEQ ID NO: 105 and a VL domain of SEQ ID NO: 106, or a VH domain of SEQ ID NO: 123 and a VL domain of SEQ ID NO: 124, or a VH domain of SEQ ID NO: 141 and a VL domain of SEQ ID NO: 142, or a VH domain of SEQ ID NO: 159 and a VL domain of SEQ ID NO: 160, or a VH domain of SEQ ID NO: 177 and a VL domain of SEQ ID NO: 178, or a VH domain of SEQ ID NO: 195 and a VL domain of SEQ ID NO: 196. Certain preferred reference antibodies are selected from antibodies 23B9-1, 24C6-1, 27A2-1, 29B5-1, 2B₁₁-1, 33B6-1, 4D11-1, 54F1-1, 69H7-1, 75C9-1 or 9E4-1, or antibodies based on these antibodies.

As the identification of competing antibodies is determined in comparison to a reference antibody, it will be understood that actually determining the epitope to which either or both antibodies bind is not in any way required in order to identify a competing antibody. However, epitope mapping can be performed using standard techniques, if desired.

In the following descriptions of the compositions, immunoconjugates, pharmaceuticals, combinations, cocktails, kits, first and second medical uses and all methods in accordance with this invention, the terms “antibody” and “immunoconjugate”, or an antigen-binding region or fragment thereof, unless otherwise specifically stated or made clear from the scientific terminology, refer to a range of anti-TRPV1 antibodies as well as to the specific antibodies described in the Example section herein.

The terms “antibody” and “immunoglobulin”, as used herein, refer broadly to any immunological binding agent that comprises an antigen binding domain, including polyclonal and monoclonal antibodies.

Thus, the term “antibody” includes immunological binding agents that comprise an antigen binding domain obtained from or derived from an antibody (or based on an antigen binding domain of an antibody), e.g. obtained from or derived from an Ig (e.g. IgG) antibody (or based on an antigen binding domain of an Ig (e.g. IgG) antibody).

In some embodiments, polyclonal antibodies are preferred, e.g. polyclonal antibodies that are generated in (or raised in or isolated from) an animal (e.g. a rabbit such as a specific pathogen free (SPF) rabbit) immunized with an isolated peptide or conjugate (preferably a conjugate) of the present invention. Preferred isolated peptides and conjugates are described elsewhere herein.

In some embodiments, monoclonal antibodies are preferred (e.g. mouse monoclonal or human monoclonal antibodies or humanized monoclonal antibodies or rabbit monoclonal antibodies). Preferred monoclonal antibodies include those based on the 23B9-1, 24C6-1, 27A2-1, 29B5-1, 2B₁₁-1, 33B6-1, 4D11-1, 54F1-1, 69H7-1, 75C9-1 or 9E4-1 antibodies of the invention (e.g. those having the CDR sequences and/or VH domain and/or VL domain sequences thereof, or sequences substantially homologous thereto).

Depending on the type of constant domain in the heavy chains, whole antibodies are assigned to one of five major classes: IgA, IgD, IgE, IgG, and IgM and the antibodies of the invention may be in any one of these classes. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG (e.g. IgG₂ or IgG₄) and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art. Diabodies, in particular, are further described in EP 404 097 and WO 93/11161; whereas linear antibodies are further described in the art.

In some embodiments, the antibodies of the invention are non-human antibodies (e.g. rabbit or rat or mouse antibodies). In some embodiments, the antibodies of the invention are rabbit antibodies.

In some embodiments, the antibodies of the invention are human antibodies, more preferably fully human antibodies. In this regard, human antibodies generally have at least two potential advantages for use in human therapy. First, the human immune system should not recognize the antibody as foreign. Second, the half-life in the human circulation will be similar to naturally occurring human antibodies, allowing smaller and less frequent doses to be given.

The term “human” as used herein in connection with antibody molecules and binding proteins first refers to antibodies and binding proteins having variable regions (e.g., V_{H}, V_{L}, CDR or FR regions) and, optionally, constant antibody regions, isolated or derived from a human repertoire or derived from or corresponding to sequences found in humans or a human repertoire, e.g., in the human germline or somatic cells.

“Human” antibodies and binding proteins further include amino acid residues not encoded by human sequences, e.g., mutations introduced by random or site directed mutations in vitro, for example mutations introduced by in vitro cloning or PCR. Particular examples of such mutations are mutations that involve conservative substitutions or other mutations in a small number of residues of the antibody or binding protein, e.g., in up to 5, 4, 3, 2 or 1 of the residues of the antibody or binding protein, preferably e.g., in up to 5, 4, 3, 2 or 1 of the residues making up one or more of the CDRs of the antibody or binding protein. Certain examples of such “human” antibodies include antibodies and variable regions that have been subjected to standard modification techniques to reduce the amount of potentially immunogenic sites.

Thus, “human” antibodies include sequences derived from and related to sequences found in humans, but which may not naturally exist within the human antibody germline repertoire in vivo. In addition, human antibodies and binding proteins include proteins comprising human consensus sequences identified from human sequences, or sequences substantially homologous to human sequences.

In addition, human antibodies and binding proteins are not limited to combinations of VH, VL, CDR or FR regions that are themselves found in combination in human antibody molecules. Thus, human antibodies and binding proteins can include or correspond to combinations of such regions that do not necessarily exist naturally in humans (e.g. are not naturally occurring antibodies).

In some embodiments, human antibodies will be fully human antibodies. “Fully human” antibodies, as used herein, are antibodies comprising “human” variable region domains and/or CDRs, without substantial non-human antibody sequences or without any non-human antibody sequences. For example, antibodies comprising human variable region domains and/or CDRs “without substantial non-human antibody sequences” are antibodies, domains and/or CDRs in which only up to 5, 4, 3, 2 or 1 amino acids are amino acids that are not encoded by human antibody sequences. Thus, “fully human” antibodies are distinguished from “humanized” antibodies, which are based on substantially non-human variable region domains, e.g., mouse variable region domains, in which certain amino acids have been changed to better correspond with the amino acids typically present in human antibodies.

The “fully human” antibodies of the invention may be human variable region domains and/or CDRs without any other substantial antibody sequences, such as being single chain antibodies. Alternatively, the “fully human” antibodies of the invention may be human variable region domains and/or CDRs integral with or operatively attached to one or more human antibody constant regions. Certain preferred fully human antibodies are IgG antibodies with the full complement of IgG constant regions.

In other embodiments, “human” antibodies of the invention will be part-human chimeric antibodies. “Part-human chimeric” antibodies, as used herein, are antibodies comprising “human” variable region domains and/or CDRs operatively attached to, or grafted onto, a constant region of a non-human species, such as rat or mouse. Such part-human chimeric antibodies may be used, for example, in pre-clinical studies, wherein the constant region will preferably be of the same species of animal used in the pre-clinical testing. These part-human chimeric antibodies may also be used, for example, in ex vivo diagnostics, wherein the constant region of the non-human species may provide additional options for antibody detection.

In some embodiments, the antibodies of the invention will be humanized antibodies. “Humanized” antibodies, which are based on substantially non-human variable region domains are antibodies in which certain amino acids have been changed to better correspond with the amino acids typically present in human antibodies. Methods for generating humanized antibodies are well known in the art. For example, humanized antibodies can be accomplished by inserting the appropriate CDRs (e.g. murine CDRs) into a human antibody “scaffold”. In some cases, one or more CDR residues may be changed to better correspond with the amino acids typically present in human antibodies.

The term “heavy chain complementarity determining region” (“heavy chain CDR”) as used herein refers to regions of hypervariability within the heavy chain variable region (VH domain) of an antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (VH domain) as used herein refers to the variable region of a heavy chain of an antibody molecule.

The term “light chain complementarity determining region” (“light chain CDR”) as used herein refers to regions of hypervariability within the light chain variable region (VL domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (VL domain) as used herein refers to the variable region of a light chain of an antibody molecule.

CDR sequences of certain antibodies of the invention are set forth herein in Tables A-K. In some other embodiments, CDR sequences of antibodies of the invention may be CDR sequences in the VH domains and VL domains of antibodies of the invention as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, MD, 647-669, 1991) or Chothia (e.g. Chothia C, et al. (1989) Nature, 342:877-883, or Al-Lazikani et al., (1997) JMB 273,927-948), or as identified using the IMGT numbering scheme (e.g. Lefranc, M.-P., The Immunologist, 7, 132-136 (1999); www.imgt.org)).

The CDRs of the antibodies of the invention are preferably separated by appropriate framework regions such as those found in naturally occurring antibodies and/or effective engineered antibodies. Appropriate framework regions (FRs) are described elsewhere herein.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In certain embodiments, the antibody or antibody fragment of the present invention comprises all or a portion of a heavy chain constant region, such as an IgG1, IgG2, IgG3, IgG4, IgA1, IgA2, IgE, IgM or IgD constant region. Preferably, the heavy chain constant region is an IgG heavy chain constant region, e.g. an IgG2 or an IgG4 heavy chain constant region, or a portion thereof. Furthermore, the antibody or antibody fragment can comprise all or a portion of a kappa light chain constant region or a lambda light chain constant region, or a portion thereof. All or part of such constant regions may be produced naturally or may be wholly or partially synthetic. Appropriate sequences for such constant regions are well known and documented in the art. When a full complement of constant regions from the heavy and light chains are included in the antibodies of the invention, such antibodies are typically referred to herein as “full length” antibodies or “whole” antibodies. Thus, in some embodiments, the antibodies of the invention are Ig (e.g. IgG) antibodies.

The antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the antibody or antibody fragment comprises an antibody light chain variable region (V L) that comprises three CDR domains and an antibody heavy chain variable region (VH) that comprises three CDR domains. Said VL and VH generally form the antigen binding site.

An “Fv” fragment is the minimum antibody fragment that contains a complete antigen-recognition and binding site. This region has a dimer of one heavy chain and one light chain variable domain in tight, non-covalent association. It is in this configuration that the three hypervariable regions (CDRs) of each variable domain interact to define an antigen-binding site on the surface of the VH-VL dimer. Collectively, the six hypervariable regions (CDRs) confer antigen-binding specificity to the antibody.

However, it is well documented in the art that the presence of three CDRs from the light chain variable domain and three CDRs from the heavy chain variable domain of an antibody is not always necessary for antigen binding. Thus, constructs smaller than the above classical antibody fragment are known to be effective.

For example, camelid antibodies have an extensive antigen binding repertoire but are devoid of light chains. Also, results with single domain antibodies comprising VH domains alone or VL domains alone show that these domains can bind to antigen with acceptably high affinities. Thus, three CDRs can effectively bind antigen.

Thus, although preferred antibodies of the invention might comprise six CDR regions (three from a light chain and three from a heavy chain), antibodies with fewer than six CDR regions (e.g. 3 CDR regions) are encompassed by the invention. Antibodies with CDRs from only the heavy chain or light chain are also contemplated.

A yet further aspect of the invention provides an antibody, preferably an isolated antibody, which binds to (or specifically recognizes) TRPV1 and which has the ability to compete with (i.e. bind to the same or substantially the same epitope as) an antibody of the invention for binding to TRPV1. For example, antibodies that can compete with antibodies (e.g. polyclonal antibodies such as those described in the Example section herein or e.g. monoclonal antibodies as described herein) that have been generated against isolated peptides (or conjugates) of the invention for binding to TRPV1 represent a further aspect of the invention. Other features and properties of other aspects of the invention apply, mutatis mutandis, to this aspect of the invention.

In some embodiments, the invention provides an antibody, preferably an isolated antibody, which binds to (or specifically recognizes) TRPV1 and which has the ability to compete with (i.e. bind to the same or substantially the same epitope as) the 23B9-1, 24C6-1, 27A2-1, 29B5-1, 2B₁₁-1, 33B6-1, 4D11-1, 54F1-1, 69H7-1, 75C9-1 and/or 9E4-1 monoclonal antibodies of the invention as described herein for binding to TRPV1. Thus, in some embodiments, the invention provides an antibody, preferably an isolated antibody, which binds to (or specifically recognizes) TRPV1 and which has the ability to compete with an antibody comprising the same VH domain and VL domain amino acid sequences as the 23B9-1, 24C6-1, 27A2-1, 29B5-1, 2B₁₁-1, 33B6-1, 4D11-1, 54F1-1, 69H7-1, 75C9-1 or 9E4-1 monoclonal antibodies of the invention (the VH and VL domain sequences of these antibodies are set out elsewhere herein) for binding to TRPV1.

In other embodiments, the invention provides an antibody, preferably an isolated antibody, which binds to or specifically recognizes TRPV1 and which has the ability to compete with an antibody comprising the same CDRs as the 23B9-1, 24C6-1, 27A2-1, 29B5-1, 2B₁₁-1, 33B6-1, 4D11-1, 54F1-1, 69H7-1, 75C9-1 or 9E4-1 monoclonal antibodies of the invention (the CDR sequences of these antibodies are set out elsewhere herein) for binding to TRPV1.

Binding to the same epitope/antigen can be readily tested by methods well known and described in the art, e.g. using binding assays such as a competition assay.

An exemplary competition assay involves assessing the binding of various effective concentrations of an antibody of the invention to TRPV1 (e.g. a rabbit polyclonal antibody that binds to an isolated peptide or epitope of TRPV1 of the invention, or e.g. an antibody selected from 23B9-1, 24C6-1, 27A2-1, 29B5-1, 2611-1, 33B6-1, 4D11-1, 54F1-1, 69H7-1, 75C9-1 or 9E4-1, or an antibody based thereon) in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to TRPV1) is evidence of binding to substantially the same epitope. Preferably, the test antibody significantly reduces the amount of antibody of the invention that binds to TRPV1. Preferably, the test antibody reduces the amount of antibody of the invention that binds to TRPV1 by at least about 60%, 65%, 70%, 75%, 80%, 85% or 95%. ELISA and flow cytometry assays may be used for assessing inhibition of binding in such a competition assay but other suitable techniques would be well known to a person skilled in the art.

The term “competing antibodies” (“or antibody that has the ability to compete with”), as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to TRPV1. Preferably, the competing antibody can bind to the same epitope as the reference antibody. Alternatively viewed, the competing antibody preferably has the same epitope specificity as the reference antibody.

“Reference antibodies” as used herein are antibodies (e.g. the polyclonal rabbit antibodies described herein, or e.g. an antibody selected from 23B9-1, 24C6-1, 27A2-1, 29B5-1, 2B₁₁-1, 33B6-1, 4D11-1, 54F1-1, 69H7-1, 75C9-1 or 9E4-1, or an antibody based thereon) that bind to an isolated peptide (preferably SEQ ID NO:2 or SEQ ID NO:3) or epitope of TRPV1 of the invention.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better, when compared to the abilities observed for prior art antibodies.

In any statistical analysis referred to herein, preferably the statistically significant difference over a relevant control or other comparative entity or measurement has a probability value of <0.1, preferably <0.05. Appropriate methods of determining statistical significance are well known and documented in the art and any of these may be used.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:17 or a sequence substantially homologous thereto,
(b) a VH CDR2 that has the amino acid sequence of SEQ ID NO:18 or a sequence substantially homologous thereto, and
(c) a VH CDR3 that has the amino acid sequence of SEQ ID NO:19 or a sequence substantially homologous thereto; and/or (preferably “and”)

wherein said light chain variable region comprises:
(d) a variable light (VL) CDR1 that has the amino acid sequence of SEQ ID NO:20 or a sequence substantially homologous thereto,
(e) a VL CDR2 that has the amino acid sequence of SEQ ID NO:21 or a sequence substantially homologous thereto, and
(f) a VL CDR3 that has the amino acid sequence of SEQ ID NO:22 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 15 or a sequence substantially homologous thereto, and/or (preferably “and”) a VL domain that has the amino acid sequence of SEQ ID NO: 16 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:35 or a sequence substantially homologous thereto,
(b) a VH CDR2 that has the amino acid sequence of SEQ ID NO:36 or a sequence substantially homologous thereto, and
(c) a VH CDR3 that has the amino acid sequence of SEQ ID NO:37 or a sequence substantially homologous thereto; and/or (preferably “and”)

wherein said light chain variable region comprises:
(d) a variable light (VL) CDR1 that has the amino acid sequence of SEQ ID NO:38 or a sequence substantially homologous thereto,
(e) a VL CDR2 that has the amino acid sequence of SEQ ID NO:39 or a sequence substantially homologous thereto, and
(f) a VL CDR3 that has the amino acid sequence of SEQ ID NO:40 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 33 or a sequence substantially homologous thereto, and/or (preferably “and”) a VL domain that has the amino acid sequence of SEQ ID NO: 34 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:53 or a sequence substantially homologous thereto,
(b) a VH CDR2 that has the amino acid sequence of SEQ ID NO:54 or a sequence substantially homologous thereto, and
(c) a VH CDR3 that has the amino acid sequence of SEQ ID NO:55 or a sequence substantially homologous thereto; and/or (preferably “and”)

wherein said light chain variable region comprises:
(d) a variable light (VL) CDR1 that has the amino acid sequence of SEQ ID NO:56 or a sequence substantially homologous thereto,
(e) a VL CDR2 that has the amino acid sequence of SEQ ID NO:57 or a sequence substantially homologous thereto, and
(f) a VL CDR3 that has the amino acid sequence of SEQ ID NO:58 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 51 or a sequence substantially homologous thereto, and/or (preferably “and”) a VL domain that has the amino acid sequence of SEQ ID NO: 52 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:71 or a sequence substantially homologous thereto,
(b) a VH CDR2 that has the amino acid sequence of SEQ ID NO:72 or a sequence substantially homologous thereto, and
(c) a VH CDR3 that has the amino acid sequence of SEQ ID NO:73 or a sequence substantially homologous thereto; and/or (preferably “and”)

wherein said light chain variable region comprises:
(d) a variable light (VL) CDR1 that has the amino acid sequence of SEQ ID NO:74 or a sequence substantially homologous thereto,
(e) a VL CDR2 that has the amino acid sequence of SEQ ID NO:75 or a sequence substantially homologous thereto, and
(f) a VL CDR3 that has the amino acid sequence of SEQ ID NO:76 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 69 or a sequence substantially homologous thereto, and/or (preferably “and”) a VL domain that has the amino acid sequence of SEQ ID NO: 70 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:89 or a sequence substantially homologous thereto,
(b) a VH CDR2 that has the amino acid sequence of SEQ ID NO:90 or a sequence substantially homologous thereto, and
(c) a VH CDR3 that has the amino acid sequence of SEQ ID NO:91 or a sequence substantially homologous thereto; and/or (preferably “and”)

wherein said light chain variable region comprises:
(d) a variable light (VL) CDR1 that has the amino acid sequence of SEQ ID NO:92 or a sequence substantially homologous thereto,
(e) a VL CDR2 that has the amino acid sequence of SEQ ID NO:93 or a sequence substantially homologous thereto, and
(f) a VL CDR3 that has the amino acid sequence of SEQ ID NO:94 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 87 or a sequence substantially homologous thereto, and/or (preferably “and”) a VL domain that has the amino acid sequence of SEQ ID NO: 88 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:107 or a sequence substantially homologous thereto,
(b) a VH CDR2 that has the amino acid sequence of SEQ ID NO:108 or a sequence substantially homologous thereto, and
(c) a VH CDR3 that has the amino acid sequence of SEQ ID NO:109 or a sequence substantially homologous thereto; and/or (preferably “and”)

wherein said light chain variable region comprises:
(d) a variable light (VL) CDR1 that has the amino acid sequence of SEQ ID NO:110 or a sequence substantially homologous thereto,
(e) a VL CDR2 that has the amino acid sequence of SEQ ID NO:111 or a sequence substantially homologous thereto, and
(f) a VL CDR3 that has the amino acid sequence of SEQ ID NO:112 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 105 or a sequence substantially homologous thereto, and/or (preferably “and”) a VL domain that has the amino acid sequence of SEQ ID NO: 106 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:125 or a sequence substantially homologous thereto,
(b) a VH CDR2 that has the amino acid sequence of SEQ ID NO:126 or a sequence substantially homologous thereto, and
(c) a VH CDR3 that has the amino acid sequence of SEQ ID NO:127 or a sequence substantially homologous thereto; and/or (preferably “and”)

wherein said light chain variable region comprises:
(d) a variable light (VL) CDR1 that has the amino acid sequence of SEQ ID NO:128 or a sequence substantially homologous thereto,
(e) a VL CDR2 that has the amino acid sequence of SEQ ID NO:129 or a sequence substantially homologous thereto, and
(f) a VL CDR3 that has the amino acid sequence of SEQ ID NO:130 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 123 or a sequence substantially homologous thereto, and/or (preferably “and”) a VL domain that has the amino acid sequence of SEQ ID NO: 124 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:143 or a sequence substantially homologous thereto,
(b) a VH CDR2 that has the amino acid sequence of SEQ ID NO:144 or a sequence substantially homologous thereto, and
(c) a VH CDR3 that has the amino acid sequence of SEQ ID NO:145 or a sequence substantially homologous thereto; and/or (preferably “and”)

wherein said light chain variable region comprises:
(d) a variable light (VL) CDR1 that has the amino acid sequence of SEQ ID NO:146 or a sequence substantially homologous thereto,
(e) a VL CDR2 that has the amino acid sequence of SEQ ID NO:147 or a sequence substantially homologous thereto, and
(f) a VL CDR3 that has the amino acid sequence of SEQ ID NO:148 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 141 or a sequence substantially homologous thereto, and/or (preferably “and”) a VL domain that has the amino acid sequence of SEQ ID NO: 142 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:161 or a sequence substantially homologous thereto,
(b) a VH CDR2 that has the amino acid sequence of SEQ ID NO:162 or a sequence substantially homologous thereto, and
(c) a VH CDR3 that has the amino acid sequence of SEQ ID NO:163 or a sequence substantially homologous thereto; and/or (preferably “and”)

wherein said light chain variable region comprises:
(d) a variable light (VL) CDR1 that has the amino acid sequence of SEQ ID NO:164 or a sequence substantially homologous thereto,
(e) a VL CDR2 that has the amino acid sequence of SEQ ID NO:165 or a sequence substantially homologous thereto, and
(f) a VL CDR3 that has the amino acid sequence of SEQ ID NO:166 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 159 or a sequence substantially homologous thereto, and/or (preferably “and”) a VL domain that has the amino acid sequence of SEQ ID NO: 160 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:179 or a sequence substantially homologous thereto,
(b) a VH CDR2 that has the amino acid sequence of SEQ ID NO:180 or a sequence substantially homologous thereto, and
(c) a VH CDR3 that has the amino acid sequence of SEQ ID NO:181 or a sequence substantially homologous thereto; and/or (preferably “and”)

wherein said light chain variable region comprises:
(d) a variable light (VL) CDR1 that has the amino acid sequence of SEQ ID NO:182 or a sequence substantially homologous thereto,
(e) a VL CDR2 that has the amino acid sequence of SEQ ID NO:183 or a sequence substantially homologous thereto, and
(f) a VL CDR3 that has the amino acid sequence of SEQ ID NO:184 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 177 or a sequence substantially homologous thereto, and/or (preferably “and”) a VL domain that has the amino acid sequence of SEQ ID NO: 178 or a sequence substantially homologous thereto.

In another aspect, the present invention provides an antibody, for example an isolated antibody, that binds to TRPV1 and that comprises at least one heavy chain variable region that comprises three CDRs and at least one light chain variable region that comprises three CDRs, wherein said heavy chain variable region comprises:
(a) a variable heavy (VH) CDR1 that has the amino acid sequence of SEQ ID NO:197 or a sequence substantially homologous thereto,
(b) a VH CDR2 that has the amino acid sequence of SEQ ID NO:198 or a sequence substantially homologous thereto, and
(c) a VH CDR3 that has the amino acid sequence of SEQ ID NO:199 or a sequence substantially homologous thereto; and/or (preferably “and”)

wherein said light chain variable region comprises:
(d) a variable light (VL) CDR1 that has the amino acid sequence of SEQ ID NO:200 or a sequence substantially homologous thereto,
(e) a VL CDR2 that has the amino acid sequence of SEQ ID NO:201 or a sequence substantially homologous thereto, and
(f) a VL CDR3 that has the amino acid sequence of SEQ ID NO:202 or a sequence substantially homologous thereto;

preferably wherein said substantially homologous sequence is a sequence containing 1, 2 or 3 amino acid substitutions compared to the given CDR sequence, or is a sequence containing conservative amino acid substitutions of the given CDR sequence.

Preferred embodiments of this aspect of the invention include antibodies comprising one or more of the antibody sequences (e.g. CDR sequences and/or VH domain and/or VL domain sequences) that are described elsewhere herein in connection with other aspects of the present invention. Thus, discussion of various features of the antibodies of other aspects of the invention and preferred embodiments apply mutatis mutandis to this aspect of the invention. In some preferred embodiments of this aspect of the invention, the invention provides an antibody comprising a VH domain that has the amino acid sequence of SEQ ID NO: 195 or a sequence substantially homologous thereto, and/or (preferably “and”) a VL domain that has the amino acid sequence of SEQ ID NO: 196 or a sequence substantially homologous thereto.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:15 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:16 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:33 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:34 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:51 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:52 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:69 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:70 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:87 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:88 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:105 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:106 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:123 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:124 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:141 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:142 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:159 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:160 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:177 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:178 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In another aspect, the present invention provides an antibody that binds to (or specifically binds to) TRPV1, said antibody comprising at least one heavy chain variable region that comprises three CDRs and at least one light chain variable region that comprises three CDRs, wherein said heavy chain variable region comprises a variable heavy (VH) CDR1, a VH CDR2 and a VH CDR3 of (or from or from within) the heavy chain variable region that has the sequence of SEQ ID NO:195 (or VH CDR1, VH CDR2 and VH CDR3 sequences substantially homologous thereto), and/or (preferably “and”) wherein said light chain variable region comprises a variable light (VL) CDR1, a VL CDR2 and a VL CDR3 of (or from or from within) the light chain variable region that has the sequence of SEQ ID NO:196 (or VL CDR1, VL CDR2 and VL CDR3 sequences substantially homologous thereto). Discussion of various features of the antibodies of other aspects of the invention (e.g. the ability to bind to an epitope of TRPV1 that is in the region of TRPV1 defined by amino acid residues 455-470 of TRPV1 (SEQ ID NO:1)) and preferred embodiments may be applied mutatis mutandis to this aspect of the invention.

In some embodiments, CDR sequences of (or from or from within) a heavy chain variable domain or a light chain variable domain are CDR sequences as set forth in Tables A-K herein. In some embodiments, CDR sequences of (or from or from within) a heavy chain variable domain or a light chain variable domain are CDR sequences as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, MD, 647-669, 1991) or Chothia (e.g. Chothia C, et al. (1989) Nature, 342:877-883, or Al-Lazikani et al., (1997) JMB 273, 927-948). CDR sequences of (or from or from within) a heavy chain variable domain or a light chain variable domain may be CDR sequences as identified using the IMGT numbering scheme (e.g. Lefranc, M.-P., The Immunologist, 7, 132-136 (1999); www.imgt.org)).

The antibodies, peptides, binding proteins and nucleic acid molecules of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins, peptides or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with a protein or polypeptide molecule such as isolated peptides, light chain CDRs 1, 2 and 3, heavy chain CDRs 1, 2 and 3, light chain variable regions, heavy chain variable regions, and binding proteins or antibodies of the invention, including full length antibodies, the term “isolated” or “purified” typically refers to a protein substantially free of cellular material or other proteins from the source from which it is derived. In some embodiments, particularly where the protein is to be administered to humans or animals, such isolated or purified proteins are substantially free of culture medium when produced by recombinant techniques, or chemical precursors or other chemicals when chemically synthesized.

The term “fragment” as used herein refers to fragments of biological relevance, e.g. fragments that contribute to antigen binding, e.g. form part of the antigen binding site, and/or contribute to the functional properties of the TRPV1 antibody. Certain preferred fragments comprise a heavy chain variable region (VH domain) and/or a light chain variable region (VL domain) of the antibodies of the invention.

A person skilled in the art will appreciate that the antibodies, antibody fragments, and immunoconjugates of the invention may be prepared in any of several ways well known and described in the art. For example, polyclonal antibodies may be prepared by immunizing an animal (non-human animal e.g. a rabbit) with an isolated peptide or conjugate of the invention and isolating (and optionally purifying) antibodies to the isolated peptide or conjugate that have been generated by the animal. In other embodiments, antibodies, antibody fragments, and immunoconjugates of the invention may be prepared by recombinant methods.

Nucleic acid fragments encoding the light and heavy chain variable regions of the antibodies of the invention can be derived or produced by any appropriate method, e.g. by cloning or synthesis.

Once nucleic acid fragments encoding the light and heavy chain variable regions of the antibodies of the invention have been obtained, these fragments can be further manipulated by standard recombinant DNA techniques, for example to convert the variable region fragments into full length antibody molecules with appropriate constant region domains, or into particular formats of antibody fragment discussed elsewhere herein, e.g. Fab fragments, scFv fragments, etc. Typically, or as part of this further manipulation procedure, the nucleic acid fragments encoding antibody molecules of the invention are generally incorporated into one or more appropriate expression vectors in order to facilitate production of the antibodies of the invention.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates a recombinant expression vector containing a nucleic acid molecule of the invention, or a fragment thereof, and the necessary regulatory sequences for the transcription and translation of the protein sequence encoded by the nucleic acid molecule of the invention.

Suitable regulatory sequences may be derived from a variety of sources, including bacterial, fungal, viral, mammalian, or insect genes and are well known in the art. Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the invention may also contain a selectable marker gene that facilitates the selection of host cells transformed or transfected with a recombinant molecule of the invention.

The recombinant expression vectors may also contain genes that encode a fusion moiety that provides increased expression of the recombinant protein; increased solubility of the recombinant protein; and aid in the purification of the target recombinant protein by acting as a ligand in affinity purification (for example appropriate “tags” to enable purification and/or identification may be present, e.g., His tags or myc tags).

Recombinant expression vectors can be introduced into host cells to produce a transformed host cell. The terms “transformed with”, “transfected with”, “transformation” and “transfection” are intended to encompass introduction of nucleic acid (e.g., a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, N Y, 1989) and other laboratory textbooks.

Suitable host cells include a wide variety of eukaryotic host cells and prokaryotic cells. For example, the proteins (e.g. antibodies) of the invention may be expressed in yeast cells or mammalian cells. In addition, the proteins of the invention may be expressed in prokaryotic cells, such as Escherichia coli.

Given the teachings provided herein, promoters, terminators, and methods for introducing expression vectors of an appropriate type into plant, avian, and insect cells may also be readily accomplished.

Alternatively, the proteins (e.g. antibodies) of the invention may also be expressed in non-human transgenic animals such as, rats, rabbits, sheep and pigs.

The proteins of the invention may also be prepared by chemical synthesis using techniques well known in the chemistry of proteins such as solid phase synthesis.

N-terminal or C-terminal fusion proteins comprising the antibodies and proteins (e.g. isolated peptides) of the invention conjugated to other molecules, such as proteins, may be prepared by fusing through recombinant techniques. The resultant fusion proteins contain an antibody or protein of the invention fused to the selected protein or marker protein, or tag protein as described herein. The antibodies and proteins of the invention may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate.

A yet further aspect provides an expression construct or expression vector comprising one or more of the nucleic acid fragments or segments or molecules of the invention. Preferably the expression constructs or vectors are recombinant. Also provided is a set of expression vectors (or a set of expression constructs) which, together (collectively), encode an antibody of the invention. Such a set of expression vectors may be characterised in that when the set is expressed (i.e. expressed together) (e.g. in a host cell) an antibody (an entire antibody) of the present invention is expressed and preferably assembled.

Preferably said constructs or vectors further comprise the necessary regulatory sequences for the transcription and translation of the protein sequence encoded by the nucleic acid molecule of the invention.

A yet further aspect provides a host cell or virus comprising one or more expression constructs or expression vectors of the invention. Also provided are host cells or viruses comprising one or more of the nucleic acid molecules of the invention. A host cell (e.g. a mammalian host cell) or virus expressing an antibody of the invention forms a yet further aspect.

A yet further aspect of the invention provides a method of producing (or manufacturing or isolating or identifying or generating) an antibody of the present invention, said method employing an isolated peptide or conjugate of the invention Alternatively viewed, the present invention provides the use of an isolated peptide or conjugate of the invention for the identification (or isolation or generation or production) of an antibody of the invention.

A yet further aspect of the invention provides a method of producing (or manufacturing or isolating or identifying or generating) an antibody of the present invention comprising a step of immunizing an animal (non-human animal e.g. a rabbit) with an isolated peptide (or conjugate) of the invention. Preferred methods include a step of obtaining from said animal antibodies that have been generated (or raised) against the isolated peptide (or conjugate) of the invention, and optionally a step of purification of the antibody product and/or formulating the antibody or product into a composition including at least one additional component, such as a pharmaceutically acceptable carrier or excipient.

A yet further aspect of the invention provides a method of identifying (or generating) an antibody of the present invention comprising a step of immunizing an animal (non-human animal, e.g. a rabbit) with an isolated peptide (or conjugate) of the invention, isolating B-cells from said immunized animal and generating B-cell clones, culturing said B-cell clones under conditions suitable for the production of antibodies, and screening (e.g. using an ELISA assay) supernatants from said cultured B-cell clones for the ability of an antibody therein to bind to said isolated peptide (or conjugate) of the invention. The ability of an antibody to bind to said isolated peptide (or conjugate) of the invention is indicative that an antibody in accordance with the invention has been identified. The VH domain and/or VL domain nucleotide sequences of an antibody produced by a given B-cell clone may be determined (e.g. using any suitable sequencing method). A preferred method is described in Example 2 herein.

A yet further aspect of the invention provides a method of producing (or manufacturing or isolating or identifying or generating) an antibody of the present invention by employing an isolated peptide or conjugate of the invention in hybridoma technology (e.g. conventional hybridoma technology). Alternatively viewed, the present invention provides the use of an isolated peptide or conjugate of the invention for the identification (or isolation or generation or production) of an antibody of the invention using hybridoma technology. In some embodiments, a non-human animal (e.g. mouse or rabbit) is immunized with an isolated peptide or conjugate of the invention, spleen cells are isolated from said immunized animal (e.g. mouse or rabbit) and fused with myeloma cells (e.g. mouse myeloma cells) lacking HGPRT expression (such myeloma cells are unable to grow in HAT containing media) and hybrid (i.e. fused or hybridoma) cells are selected using hypoxanthine, aminopterin and thymine (HAT) containing media. Only fused cells grow in HAT containing media.

A yet further aspect of the invention provides a method of identifying (or isolating or generating) an antibody of the invention which employs phage display technology (with a phage display antibody library). Alternatively viewed, the present invention provides the use of an isolated peptide or conjugate of the invention for the identification (or isolation or generation or production) of an antibody of the invention using phage display technology (with a phage display antibody library). In some embodiments, an isolated peptide or conjugate of the invention (typically immobilised on a solid support such as a bead or microbead or plate or microtitre plate) is contacted with phage library (bacteriophage library typically a filamentous bacteriophage library such as an M13 of fd phage library) which displays (or presents or expresses) on the phage surface a library of antibodies or antibody fragments such as scFv or Fab fragments. Any suitable phage display antibody library may be used and the skilled person is familiar with these (and e.g. there are commercially available phage display antibody libraries). The bound phage is then eluted and the identity of the displayed antibody may be readily determined by isolating and sequencing the phage's nucleic acid (or at least the portion of the nucleic acid that encodes the displayed antibody). In some embodiments, after elution of the bound phage, one or more (e.g. 1, 2, 3, 4, 5 or more) additional rounds of contacting and eluting is performed prior to identifying the displayed antibody of the bound phage. Such additional rounds typically further enrich the library. In some typical embodiments, where the peptide or conjugate of the invention is immobilised on a solid support, the solid support is typically washed after the contacting step (and prior to the eluting step), with those phage displaying antibodies or antibody fragments that bind to the immobilised isolated peptide or conjugate remaining bound (and the others being washed away).

A yet further aspect of the invention provides a method of producing (or manufacturing) an antibody of the present invention comprising a step of culturing the host cells of the invention. Preferred methods comprise the steps of (i) culturing a host cell comprising one or more of the recombinant expression vectors (or a set of expression vectors) or one or more of the nucleic acid sequences (or a set of nucleic acid molecules) of the invention under conditions suitable for the expression of the encoded antibody; and optionally (ii) isolating or obtaining the antibody from the host cell or from the growth medium/supernatant.

In embodiments when the antibody or protein of the invention is made up of more than one polypeptide chain (e.g. certain fragments such as Fab fragments or whole antibodies), then all the polypeptides are preferably expressed in the host cell, either from the same or a different expression vector, so that the complete proteins, e.g. antibody proteins of the invention, can assemble in the host cell and be isolated or purified therefrom.

In some embodiments, methods of producing (or manufacturing or isolating or identifying or generating) an antibody in accordance with the invention may also comprise a step of purification of the antibody or protein product and/or formulating the antibody or product into a composition including at least one additional component, such as a pharmaceutically acceptable carrier or excipient.

In another aspect, the invention provides a method of binding TRPV1, comprising contacting a composition comprising TRPV1 with an antibody of the invention, or an immunoconjugate thereof.

In yet another aspect, the invention provides a method of detecting TRPV1, comprising contacting a composition suspected of containing TRPV1 with an antibody of the invention, or an immunoconjugate thereof, under conditions effective to allow the formation of TRPV1/antibody complexes and detecting the complexes so formed.

Testing the ability of one or more antibodies to bind to TRPV1 can be carried out by any appropriate method, which are well known and described in the art. Suitable methods are also described in the Examples section.

The invention also provides a range of conjugated antibodies and fragments thereof in which the anti-TRPV1 antibody is operatively attached to at least one other therapeutic agent. The term “immunoconjugate” is broadly used to define the operative association of the antibody with another effective agent (e.g. therapeutic agent) and is not intended to refer solely to any type of operative association, and is particularly not limited to chemical “conjugation”. Recombinant fusion proteins are particularly contemplated. So long as the delivery or targeting agent is able to bind to the target and the therapeutic or diagnostic agent is sufficiently functional upon delivery, the mode of attachment will be suitable.

In some embodiments, antibodies of the invention are used (e.g. used therapeutically) in their “naked” unconjugated form.

Compositions comprising at least a first antibody of the invention or an immunoconjugate thereof constitute a further aspect of the present invention. Formulations (compositions) comprising one or more antibodies of the invention in admixture with a suitable diluent, carrier or excipient constitute a preferred embodiment of the present invention. Such formulations may be for pharmaceutical use and thus compositions of the invention are preferably pharmaceutically acceptable. Suitable diluents, excipients and carriers are known to the skilled man.

The compositions according to the invention may be presented, for example, in a form suitable for oral, nasal, parenteral, intraperitoneal, intravenal, topical or rectal administration. In some embodiments, a form suitable for intravenal administration is preferred.

The active compounds defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Nasal sprays may be formulated similarly in aqueous solution and packed into spray containers, either with an aerosol propellant or provided with means for manual compression.

The pharmaceutical compositions (formulations) of the present invention are preferably administered parenterally. Intravenous administration is preferred in some embodiments. Parenteral administration may be performed by subcutaneous, intramuscular or intravenous injection by means of a syringe. Alternatively, parenteral administration can be performed by means of an infusion pump. A further option is a composition which may be a powder or a liquid for the administration of the antibody in the form of a nasal or pulmonal spray. As a still further option, the antibodies of the invention can also be administered transdermally, e.g. from a patch, optionally an iontophoretic patch, or transmucosally, e.g. bucally.

Suitable dosage units can be determined by a person skilled in the art.

The pharmaceutical compositions may additionally comprise further active ingredients in the context of co-administration regimens or combined regimens.

A further aspect of the present invention provides the anti-TRPV1 antibodies of the invention for use in therapy, in particular for use in pain therapy (or management of pain).

In some embodiments, the pain is acute pain. In some embodiments, the pain is chronic pain.

In some embodiments, the acute pain is acute post-operative pain or acute post-traumatic pain.

In some embodiments, the chronic pain is nociceptive pain, for example osteoarthritis associated pain, arthritis associated pain, low back pain or neck pain.

In some embodiments, the chronic pain is neuropathic pain, for example painful diabetic polyneuropathy, postherpetic neuralgia, post traumatic neuralgia or radiculopathy. In some embodiments, the chronic pain is painful bladder syndrome, vulvodynia, chronic pancreatitis or visceral pain.

In some embodiments, the pain is itch (either acute or chronic itch).

In some embodiments, the pain is selected from the group consisting of inflammatory pain, idiopathic pain and neuropathic pain.

“Therapy” includes treatment and prophylaxis, i.e. in includes both treatment and preventative uses. “Pain therapy” thus includes the treatment of pain and the prevention of pain.

Thus, in some embodiments, the present invention provides the anti-TRPV1 antibodies of the invention for use in the treatment of pain. Thus, in some embodiments, the present invention provides the anti-TRPV1 antibodies of the invention for use in the prevention of pain.

In some embodiments, pain to be treated (or prevented) in accordance with the present invention is pain that is associated with (or caused by, or characterised by) activation of TPRV1 by a stimulus that has the same (or an analogous) mechanism of activating of TRPV1 as capsaicin (e.g. activation by structural and/or functional analogues of capsaicin). In some embodiments, pain to be treated (or prevented) in accordance with the present invention is pain that is associated with (or caused by, or characterised by) activation of TPRV1 by one or more endogenous ligands (or endogenous activators). Such endogenous ligands may be lipidic compounds, for example lipidic compounds produced during inflammation or tissue injury (e.g. anandamide, metabolites of lipoxygenase and lysophosphatidic acid (LPA)). In some embodiments, the endogenous ligand is a structural and/or functional analogue of capsaicin.

In some embodiments, pain to be treated (or prevented) in accordance with the present invention is pain that is inducible by capsaicin or by analogues of capsaicin.

A further aspect of the present invention provides the anti-TRPV1 antibodies of the invention for use in inhibiting activation of TRPV1, e.g. for use in inhibiting activation of TPRV1 by a stimulus that has the same (or analogous) mechanism of activating of TRPV1 as capsaicin (e.g. activation by structural and/or functional analogues of capsaicin).

In another aspect, the present invention provides immunoconjugates of the invention for use in therapy, in particular for use in the treatment of pain.

The in vivo methods and uses as described herein are generally carried out in a mammal. Any mammal may be treated, for example humans and any livestock, domestic or laboratory animal. Specific examples include mice, rats, pigs, cats, dogs, sheep, rabbits, cows and monkey. Preferably, however, the mammal is a human.

Thus, the term “animal” or “patient” as used herein includes any mammal, for example humans and any livestock, domestic or laboratory animal. Specific examples include mice, rats, pigs, cats, dogs, sheep, rabbits, cows and monkey. Preferably, however, the animal or patient is a human subject. Thus, subjects or patients treated in accordance with the present invention will preferably be humans.

In some embodiments, subjects or patients will be those having pain (or suffering from or experiencing pain), or those at risk of having pain or at risk of developing pain.

In some embodiments, the subject to be treated has a tissue in which TRPV1 has been up-regulated. In such cases, the subject may have a high (or higher) pain awareness.

In some embodiments, the subject to be treated has a tissue in which TRPV1 has been sensitized such that the subject has a low (or lower) pain threshold.

Alternatively viewed, the present invention provides a method of treating pain which method comprises administering to a patient in need thereof a therapeutically effective amount of an antibody of the invention as defined herein. Embodiments of the therapeutic uses of the invention described herein apply, mutatis mutandis, to this aspect of the invention.

The present invention also provides a method of treating a disease that is characterized by activation of TPRV1, e.g. activation by a stimulus that has the same (or an analogous) mechanism of activating of TRPV1 as capsaicin (e.g. activation by structural and/or functional analogues of capsaicin), which method comprises administering to a patient in need thereof a therapeutically effective amount of an antibody of the invention as defined herein. Embodiments of the therapeutic uses of the invention described herein apply, mutatis mutandis, to this aspect of the invention.

A therapeutically effective amount will be determined based on the clinical assessment and can be readily monitored.

Further alternatively viewed, the present invention provides the use of an antibody of the invention as defined herein in the manufacture of a medicament for use in therapy. Preferred therapy is pain therapy as described elsewhere herein. Embodiments of the therapeutic uses of the invention described herein apply, mutatis mutandis, to this aspect of the invention.

Further alternatively viewed, the present invention provides the use of an antibody of the invention as defined herein for the treatment of a disease that is characterised by activation of TPRV1, e.g. activation by a stimulus that has the same (or analogous) mechanism of activating of TRPV1 as capsaicin (e.g. activation by structural and/or functional analogues of capsaicin). A preferred use is for the treatment of pain. Embodiments of the therapeutic uses of the invention described herein apply, mutatis mutandis, to this aspect of the invention.

The antibodies and compositions and methods and uses of the present invention may be used in combination with other therapeutics and diagnostics. In terms of biological agents, preferably diagnostic or therapeutic agents, for use “in combination” with an anti-TRPV1 antibody in accordance with the present invention, the term “in combination” is succinctly used to cover a range of embodiments. The “in combination” terminology, unless otherwise specifically stated or made clear from the scientific terminology, thus applies to various formats of combined compositions, pharmaceuticals, cocktails, kits, methods, and first and second medical uses.

The “combined” embodiments of the invention thus include, for example, where an anti-TRPV1 antibody of the invention is a naked antibody and is used in combination with an agent or therapeutic agent that is not operatively attached thereto. In other “combined” embodiments of the invention, an anti-TRPV1 antibody of the invention is an immunoconjugate wherein the antibody is itself operatively associated or combined with the agent or therapeutic agent. The operative attachment includes all forms of direct and indirect attachment as described herein and known in the art.

The “combined” uses, particularly in terms of an anti-TRPV1 antibody of the invention in combination with therapeutic agents, also include combined compositions, pharmaceuticals, cocktails, kits, methods, and first and second medical uses wherein the therapeutic agent is in the form of a prodrug. In such embodiments, the activating component able to convert the prodrug to the functional form of the drug may again be operatively associated with the anti-TRPV1 antibodies of the present invention.

Thus, where combined compositions, pharmaceuticals, cocktails, kits, methods, and first and second medical uses are described, preferably in terms of diagnostic agents, and more preferably therapeutic agents, the combinations include anti-TRPV1 antibodies that are naked antibodies and immunoconjugates, and wherein practice of the in vivo embodiments of the invention involves the prior, simultaneous or subsequent administration of the naked antibodies or immunoconjugate and the biological, diagnostic or therapeutic agent; so long as, in some conjugated or unconjugated form, the overall provision of some form of the antibody and some form of the biological, diagnostic or therapeutic agent is achieved.

The foregoing and other explanations of the effects of the present invention are made for simplicity to explain the combined mode of operation, type of attached agent(s) and such like. This descriptive approach should not be interpreted as either an understatement or an oversimplification of the beneficial properties of the anti-TRPV1 antibodies of the invention. It will therefore be understood that such antibodies themselves have anti-TRPV1 properties and that immunoconjugates of such antibodies will maintain these properties and combine them with the properties of the attached agent; and further, that the combined effect of the antibody and any attached agent will typically be enhanced and/or magnified.

The invention therefore provides compositions, pharmaceutical compositions, therapeutic kits and medicinal cocktails comprising, optionally in at least a first composition or container, a biologically effective amount of at least a first anti-TRPV1 antibody of the invention, or an antigen-binding fragment or immunoconjugate of such an anti-TRPV1 antibody; and a biologically effective amount of at least a second biological agent, component or system.

The “at least a second biological agent, component or system” will often be a therapeutic or diagnostic agent, component or system, but it need not be. For example, the at least a second biological agent, component or system may comprise components for modification of the antibody and/or for attaching other agents to the antibody.

Where therapeutic or diagnostic agents are included as the at least a second biological agent, component or system, such therapeutics and/or diagnostics will typically be those for use in connection with the treatment or diagnosis of one or more of the disorders defined above.

Thus, in certain embodiments “at least a second therapeutic agent” will be included in the therapeutic kit or cocktail. The term is chosen in reference to the anti-TRPV1 antibody of the invention being the first therapeutic agent.

In certain embodiments of the present invention, the second therapeutic agent may be a further pain therapy agent or an agent for the treatment of disease that is associated with (or characterised by or causes) pain.

In terms of compositions, kits and/or medicaments of the invention, the combined effective amounts of the therapeutic agents may be comprised within a single container or container means, or comprised within distinct containers or container means. The cocktails will generally be admixed together for combined use. Agents formulated for intravenous administration will often be preferred. Imaging components may also be included. The kits may also comprise instructions for using the at least a first antibody and the one or more other biological agents included.

Speaking generally, the at least a second therapeutic agent may be administered to the animal or patient substantially simultaneously with the anti-TRPV1 antibody of the invention; such as from a single pharmaceutical composition or from two pharmaceutical compositions administered closely together.

Alternatively, the at least a second therapeutic agent may be administered to the animal or patient at a time sequential to the administration of the anti-TRPV1 antibody of the invention. “At a time sequential”, as used herein, means “staggered”, such that the at least a second therapeutic agent is administered to the animal or patient at a time distinct to the administration of the anti-TRPV1 antibody of the invention. Generally, the two agents are administered at times effectively spaced apart to allow the two agents to exert their respective therapeutic effects, i.e., they are administered at “biologically effective time intervals”. The at least a second therapeutic agent may be administered to the animal or patient at a biologically effective time prior to the anti-TRPV1 antibody of the invention, or at a biologically effective time subsequent to that therapeutic.

A yet further aspect is a method of imaging of a subject or sample comprising the administration of an appropriate amount of an antibody or other protein of the invention as defined herein to the subject or sample and detecting the presence and/or amount and/or the location of the antibody or other protein of the invention in the subject or sample.

For use in the imaging applications, the antibodies of the invention may be labeled with a detectable marker such as a radio-opaque or radioisotope, such as ³H, ¹⁴C, ³²P, ³⁵S, ¹²³I, ¹²⁵I, ¹³¹I; a radioactive emitter (e.g. α, β or γ emitters); a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion; or a chemical moiety such as biotin which may be detected by binding to a specific cognate detectable moiety, e.g. labelled avidin/streptavidin. Methods of attaching a label to a binding protein, such as an antibody or antibody fragment, are known in the art. Such detectable markers allow the presence, amount or location of binding protein-antigen complexes in the test sample to be examined.

The invention also includes imaging agents comprising an antibody of the invention attached to a label that produces a detectable signal, directly or indirectly. Appropriate labels are described elsewhere herein.

The invention further includes kits comprising one or more of the isolated peptides (isolated epitopes), antibodies, immunoconjugates or compositions of the invention or one or more of the nucleic acid molecules encoding the antibodies of the invention, or one or more recombinant expression vectors comprising the nucleic acid sequences of the invention, or one or more host cells or viruses comprising the recombinant expression vectors or nucleic acid sequences of the invention. Preferably said kits are for use in the methods and uses as described herein, e.g. the therapeutic, methods as described herein, or are for use in the in vitro assays or methods as described herein. The antibody in such kits may be a “naked” antibody or may be an antibody conjugate as described elsewhere herein, e.g. may be an immunoconjugate. Preferably said kits comprise instructions for use of the kit components. Preferably said kits are for treating diseases as described elsewhere herein, and optionally comprise instructions for use of the kit components to treat such diseases.

The antibodies of the invention as defined herein may also be used as molecular tools for in vitro or in vivo applications and assays. As the antibodies have an antigen binding site, these can function as members of specific binding pairs and these molecules can be used in any assay where the particular binding pair member is required.

Thus, yet further aspects of the invention provide a reagent that comprises an antibody of the invention as defined herein and the use of such antibodies as molecular tools, for example in in vitro or in vivo assays.

List of Amino Acid Sequences Disclosed Herein and their Sequence Identifiers (SEQ ID NOs)

[symbol]

[symbol]

MKKWSSTDLGAAADPLQKDTCPDPLDGDPNSRPPPAKPQLSTAKSRTRL
FGKGDSEEAFPVDCPHEEGELDSCPTITVSPVITIQRPGDGPTGARLLS
QDSVAASTEKTLRLYDRRSIFEAVAQNNCQDLESLLLFLQKSKKHLTDN
EFKDPETGKTCLLKAMLNLHDGQNTTIPLLLEIARQTDSLKELVNASYT
DSYYKGQTALHIAIERRNMALVTLLVENGADVQAAAHGDFFKKTKGRPG
FYFGELPLSLAACTNQLGIVKFLLQNSWQTADISAFDSVGNTVLHALVE
VADNTADNTKFVTSMYNEILMLGAKLHPTLKLEELTNKKGMTPLALAAG
TGKIGVLAYILQREIQEPECRHLSRKFTEWAYGPVHSSLYDLSCIDTCE
KNSVLEVIAYSSSETPNRHDMLLVEPLNRLLQDKWDRFVKRIFYFNFLV
YCLYMIIFTMAAYYRPVDGLPPFKMEKTGDYFRVTGEILSVLGGVYFFF
RGIQYFLQRRPSMKTLFVDSYSEMLFFLQSLFMLATVVLYFSHLKEYVA
SMVFSLALGWTNMLYYTRGFQQMGIYAVMIEKMILRDLCRFMFVYIVFL
FGFSTAVVTLIEDGKNDSLPSESTSHRWRGPACRPPDSSYNSLYSTCLE
LFKFTIGMGDLEFTENYDFKAVFIILLLAYVILTYILLLNMLIALMGET
VNKIAQESKNIWKLQRAITILDTEKSFLKCMRKAFRSGKLLQVGYTPDG
KDDYRWCFRVDEVNWTTWNTNVGIINEDPGNCEGVKRTLSFSLRSSRVS
GRHWKNFALVPLLREASARDRQSAQPEEVYLRQFSGSLKPEDAEVFKSP
AASGEK

[symbol]

[symbol]

[symbol]

[symbol]

[symbol]

[symbol]

YRPVDGLPPFKMEKTG

(The amino acid sequence of SEQ ID NO:2 corresponds to residues 455-470 of human TRPV1 (SEQ ID NO:1)).

[symbol]

[symbol]

[symbol]

[symbol]

[symbol]

[symbol]

(Pra-)CYRPVDGLPPFKMEKTGC(-CONH₂)

As compared to SEQ ID NO:2, this peptide has an additional cysteine residue at the N-terminus and at the C-terminus. The peptide is also amidated at the C-terminus and has a Propargyl group (Pra-) at the N-terminus. This peptide is cyclic, with the peptide being connected (cyclized) via the terminal cysteine residues. The Propargyl group provides a means for attaching the peptide to a peptide carrier (e.g. KLH).

[symbol]

[symbol]

[symbol]

[symbol]

[symbol]

[symbol]

[symbol]

YRPVDGLPPFKMEKTE

[symbol]

[symbol]

[symbol]

[symbol]

[symbol]

[symbol]

(Pra-)CYRPVDGLPPFKMEKTEC(-CONH₂)

As compared to SEQ ID NO:4, this peptide has an additional cysteine residue at the N-terminus and at the C-terminus. The peptide is also amidated at the C-terminus and has a Propargyl group (Pra-) at the N-terminus. This peptide is cyclic, with the peptide being connected (cyclized) via the terminal cysteine residues. The Propargyl group provides a means for attaching the peptide to a peptide carrier (e.g. KLH).

[symbol]

[symbol]

[symbol]

[symbol]

[symbol]

[symbol]

YRPVEGLPPYKLKNTG

[symbol]

[symbol]

[symbol]

[symbol]

[symbol]

[symbol]

(Pra-)CYRPVEGLPPYKLKNTGC(-CONH₂)

As compared to SEQ ID NO:6, this peptide has an additional cysteine residue at the N-terminus and at the C-terminus. The peptide is also amidated at the C-terminus and has a Propargyl group (Pra-) at the N-terminus. This peptide is cyclic, with the peptide being connected (cyclized) via the terminal cysteine residues. The Propargyl group provides a means for attaching the peptide to a peptide carrier (e.g. KLH).

[symbol]

[symbol]

[symbol]

[symbol]

[symbol]

[symbol]

[symbol]

YRPVEGLPPYKLNNTVG

[symbol]

[symbol]

[symbol]

[symbol]

[symbol]

[symbol]

(Pra-)CYRPVEGLPPYKLNNTVGC(-CONH₂)

As compared to SEQ ID NO:8, this peptide has an additional cysteine residue at the N-terminus and at the C-terminus. The peptide is also amidated at the C-terminus and has a Propargyl group (Pra-) at the N-terminus. This peptide is cyclic, with the peptide being connected (cyclized) via the terminal cysteine residues. The Propargyl group provides a means for attaching the peptide to a peptide carrier (e.g. KLH).

[symbol]

[symbol]

[symbol]

MKKWSSTDSGAAADPLQQDACPDPPDGDPNSRPPPAKPQLSTAKSRTRL
FGKGDSEEAFLVDCPHEEGELASCPTITVSPVITIQRPGDGPTGARLPS
QDSVATSTERTLRLYDRRSIFEAVAQNNCQDLESLLLFLQKSEKHLTDN
EFKDPETGKTCLLKAMLNLHDGQNDTIPLLLEIARQTGSLKEFVNASYT
DSYYKGQTALHIAIERRNMGLVTLLVENGADVQAAANGDFFKKTKGRPG
FYFGELPLSLAACTNQLGIVKFLLQNSWQPADISARDSVGNTVLHALVE
VADNTADNTKFVTSMYNEILILGAKLHPTLKLEELTNKKGMTPLALAAG
TGKIGVLAYILQREIQEPECRHLSRKFTEWAYGPVHSSLYDLSCIDTCE
KNSVLEVIAYSSSETPNRHDMLLVEPLNRLLQDKWDRFVKRTFYFNFLV
YCLYMIIFTMAAYYRPVDGLPPFKMEKTEDYFRVTGEILSVLGGVYFFF
RGIQYFLQRRPSMKTLFVDSYSEMLFFLQSLFMLATVVLYFSHLKEYVA
SMVFSLALGWTNMLYYTRGFQQMGIYAVMIEKMILRDLCRFMFVYVVFL
FGFSTAVVTLIEDGKNNSLPSESTSHRWRGPGCRPPDSSYNSLYSTCLE
LFKFTIGMGDLEFTENYDFKAVFIILLLAYVILTYILLLNMLIALMGET
VNKIAQESKNIWKLQRAITILDTEKSFLKCMRKAFRSGKLLQVGYTPDG
KDDYRWCFRVDEVNWTTWNTNVGIINEDPGNCEGVKRTLSFSLRSSRVS
ARHWKNFALVPLLREASARDRPSAQPEEVHLRQFSGSLRPEDAEVFKSP
PASGEK

[symbol]

[symbol]

MEKWASLDSDESEPPAQENSCPDPPDRDPNSKPPPAKPHIFATRSRTRL
FGKGDSEEASPMDCPYEEGGLASCPIITVSSVVTLQRSVDGPTCLRQTS
QDSVSTGVETPPRLYDRRSIFDAVAQSNCQELESLLSFLQKSKKRLTDS
EFKDPETGKTCLLKAMLNLHNGQNDTIALLLDIARKTDSLKQFVNASYT
DSYYKGQTALHIAIERRNMALVTLLVENGADVQAAANGDFFKKTKGRPG
FYFGELPLSLAACTNQLAIVKFLLQNSWQPADISARDSVGNTVLHALVE
VADNTADNTKFVTNMYNEILILGAKLHPTLKLEELTNKKGLTPLALAAS
SGKIGVLAYILQREIHEPECRHLSRKFTEWAYGPVHSSLYDLSCIDTCE
KNSVLEVIAYSSSETPNRHDMLLVEPLNRLLQDKWDRFVKRIFYFNFFV
YCLYMIIFTTAAYYRPVEGLPPYKLNNTVGDYFRVTGEILSVSGGVYFF
FRGIQYFLQRRPSLKSLFVDSYSEILFFVQSLFMLVSVVLYFSHRKEYV
ASMVFSLAMGWTNMLYYTRGFQQMGIYAVMIEKMILRDLCRFMFVYLVF
LFGFSTAVVTLIEDGKNNSLPVESPPHKCRGSACRPGNSYNSLYSTCLE
LFKFTIGMGDLEFTENYDFKAVFIILLLAYVILTYILLLNMLIALMGET
VNKIAQESKNIWKLQRAITILDTEKSFLKCMRKAFRSGKLLQVGFTPDG
KDDFRWCFRVDEVNWTTWNTNVGIINEDPGNCEGVKRTLSFSLRSGRVS
GRNWKNFALVPLLRDASTRDRHSTQPEEVQLKHYTGSLKPEDAEVFKDS
MAPGEK

[symbol]

[symbol]

MEQRASLDSEESESPPQENSCLDPPDRDPNCKPPPVKPHIFTTRSRTRL
FGKGDSEEASPLDCPYEEGGLASCPIITVSSVLTIQRPGDGPASVRPSS
QDSVSAGEKPPRLYDRRSIFDAVAQSNCQELESLLPFLQRSKKRLTDSE
FKDPETGKTCLLKAMLNLHNGQNDTIALLLDVARKTDSLKQFVNASYTD
SYYKGQTALHIAIERRNMTLVTLLVENGADVQAAANGDFFKKTKGRPGF
YFGELPLSLAACTNQLAIVKFLLQNSWQPADISARDSVGNTVLHALVEV
ADNTVDNTKFVTSMYNEILILGAKLHPTLKLEEITNRKGLTPLALAASS
GKIGVLAYILQREIHEPECRHLSRKFTEWAYGPVHSSLYDLSCIDTCEK
NSVLEVIAYSSSETPNRHDMLLVEPLNRLLQDKWDRFVKRIFYFNFFVY
CLYMIIFTAAAYYRPVEGLPPYKLKNTVGDYFRVTGEILSVSGGVYFFF
RGIQYFLQRRPSLKSLFVDSYSEILFFVQSLFMLVSVVLYFSQRKEYVA
SMVFSLAMGWTNMLYYTRGFQQMGIYAVMIEKMILRDLCRFMFVYLVFL
FGFSTAVVTLIEDGKNNSLPMESTPHKCRGSACKPGNSYNSLYSTCLEL
FKFTIGMGDLEFTENYDFKAVFIILLLAYVILTYILLLNMLIALMGETV
NKIAQESKNIWKLQRAITILDTEKSFLKCMRKAFRSGKLLQVGFTPDGK
DDYRWCFRVDEVNWTTWNTNVGIINEDPGNCEGVKRTLSFSLRSGRVSG
RNWKNFALVPLLRDASTRDRHATQQEEVQLKHYTGSLKPEDAEVFKDSM
VPGEK

TABLE A
Sequences of antibody 23B9-1

| | | |
|---|---|---|
| SEQ ID | | |
| NO: | Description | Sequence |

23B9-1

| | | |
|---|---|---|
| 13 | VH domain | CAGTCGGTGGAGGAGTCCGG |
| | (nucleotide) | GGGTCGCCCGGTCACGCCTG |
| | | GGACACCCCTGACACTCACC |
| | | TGCACAGTCTCTGGTATCGA |
| | | CCTCAGTAGGTATGCCGTGG |
| | | ACTGGGTCCGCCAGGCTCCA |
| | | GGGAAGGGGCTGGAATGGAT |
| | | CGGAATCATTCTTACTAGTG |
| | | GTAAGATATCATACGCGAGC |
| | | TGGGTGAAAGGCCGATTCAC |
| | | CATCTCCAAAACCTCGACCA |
| | | CGGTGGATCTGAAAATGACC |
| | | AGTCTGACAACCGAGGACAC |
| | | GGCCACTTATTTCTGTGCCA |
| | | GACATTTTGATTTGTGGGGC |
| | | CAAGGCACCCTGGTCACCGT |
| | | CTCCTCA |
| 14 | VL domain | CAAGTGCTGACCCAGACTGC |
| | (nucleotide) | ATCGCCCGTGTCTGCGGCTG |
| | | TGGGAGGCACAGTCACCATC |
| | | AACTGCCAGTCCAGTCAGAG |
| | | TGTTTATAATAACAACAACT |
| | | TAGCCTGGTATCAGCAGAAA |
| | | CCAGGGCAGCCTOCCAAGCT |
| | | CCTGATCTATGATTCATCCA |
| | | AACTGGAATCAGGGGTCCCA |
| | | TCGCGGTTTAAAGGCAGTGG |
| | | ATCTGGGACACAGTTCACTC |
| | | TCATCATCAGCGACCTGGAG |
| | | TGTGACGATGCTGCCACTTA |
| | | TTACTGTGCAGGCGGTTATA |
| | | GTGGTGTTATTGCTAGTTTC |
| | | GGCGGAGGGACCGAGGTGGT |
| | | GGTCAAA |
| 15 | VH domain | QSVEESGGRPVTPGTPLTLT |
| | (amino | CTVSGIDLSRYAVDWVRQAP |
| | acid) | GKGLEWIGIILTSGKISYAS |
| | | WVKGRFTISKTSTTVDLKMT |
| | | SLTTEDTATYFCARHFDLWG |
| | | QGTLVTVSS |
| 16 | VL domain | QVLTQTASPVSAAVGGTVTI |
| | (amino | NCQSSQSVYNNNNLAWYQQK |
| | acid) | PGQPPKLLIYDSSKLESGVP |
| | | SRFKGSGSGTQFTLIISDLE |
| | | CDDAATYYCAGGYSGVIASF |
| | | GGGTEVVVK |
| 17 | Heavy CDR1 | RYAVD |
| 18 | Heavy CDR2 | IILTSGKISYASWVKG |
| 19 | Heavy CDR3 | HFDL |
| or | | |
| 91 | | |
| 20 | Light CDR1 | QSSQSVYNNNNLA |
| 21 | Light CDR2 | DSSKLES |
| 22 | Light CDR3 | AGGYSGVIAS |
| 23 | Heavy FR1 | QSVEESGGRPVTPGTPLTLT |
| | | CTVSGIDLS |
| 24 | Heavy FR2 | WVRQAPGKGLEWIG |
| 25 | Heavy FR3 | RFTISKTSTTVDLKMTSLTT |
| | | EDTATYFCAR |
| 26 | Heavy FR4 | WGQGTLVTVSS |
| 27 | Light FR1 | QVLTQTASPVSAAVGGTVTI |
| | | NC |
| 28 | Light FR2 | WYQQKPGQPPKLLIY |
| 29 | Light FR3 | GVPSRFKGSGSGTQFTLIIS |
| | | DLECDDAATYYC |
| 30 | Light FR4 | FGGGTEVVVK |

TABLE B
Sequences of antibody 24C6-1

| | | |
|---|---|---|
| SEQ ID | | |
| NO: | Description | Sequence |

24C6-1

| | | |
|---|---|---|
| 31 | VH domain | CAGTCGGTGGAGGAGTCCGG |
| | (nucleotide) | GGGTCGCCTGGTAACGCCTG |
| | | GGACACCACTGACACTCACC |
| | | TGCACAGTCTCTGGATTCTC |
| | | TCTTAGTACCTATGCGATGG |
| | | CCTGGGTCCGCCAGGCTCCG |
| | | GGGAAGGGTCTGGAGTGGAT |
| | | CGGAATCATTGGTCAGAGTT |
| | | CTAAAACATACTACGCGATT |
| | | TGGGCGAAAGGCCGATTCAC |
| | | CATCTCCAGAACCTCGACCA |
| | | CGGTGGATCTGAAGATCCCC |
| | | AGTCCGACAACCGAGGACAC |
| | | GGCCACCTATTTCTGTGCCA |
| | | GAGGGAGTACTTATGTTTTG |
| | | GACATGTGGGGCCCGGGCAC |
| | | CCTGGTCACCGTCTCCTTA |
| 32 | VL domain | GCCGTCGTGATGACCCAGAC |
| | (nucleotide) | TGCATCCCCCGTGTCTGCAG |
| | | CTGTGGGAGGCACAGTCACC |
| | | ATCAAGTGCCAGGCCAGTCA |
| | | GAGCATTAGTAGCTACTTAG |
| | | CCTGGTATCAACAGAAACCA |
| | | GGGCAGCCTCCCAAGCTCCT |
| | | GATCGCTGATGCATCGAAGC |
| | | TGGTATCTGGGGTCCCATCG |
| | | CGTTTCAAAGGCAGTGGATC |
| | | TGGGACAGAATTCACTCTCA |
| | | CCATCAGCGACCTGGAGTGT |
| | | GCCGATGCTGCCACTTACTA |
| | | CTGTCAGAACTATTATGGTT |
| | | CTTCTGATTTTGGTACTTTC |
| | | GGCGGAGGGACCGAGGTGGT |
| | | GGTCGAG |
| 33 | VH domain | QSVEESGGRLVTPGTPLTLT |
| | (amino | CTVSGFSLSTYAMAWVRQAP |
| | acid) | GKGLEWIGIIGQSSKTYYAI |
| | | WAKGRFTISRTSTTVDLKIP |
| | | SPTTEDTATYFCARGSTYVL |
| | | DMWGPGTLVTVSL |
| 34 | VL domain | AVVMTQTASPVSAAVGGTVT |
| | (amino | IKCQASQSISSYLAWYQQKP |
| | acid) | GQPPKLLIADASKLVSGVPS |
| | | RFKGSGSGTEFTLTISDLEC |
| | | ADAATYYCQNYYGSSDFGTF |
| | | GGGTEVVVE |
| 35 | Heavy CDR1 | TYAMA |
| 36 | Heavy CDR2 | IIGQSSKTYYAIWAKG |
| 37 | Heavy CDR3 | GSTYVLDM |
| 38 | Light CDR1 | QASQSISSYLA |
| 39 | Light CDR2 | DASKLVS |
| 40 | Light CDR3 | QNYYGSSDFGT |
| 41 | Heavy FR1 | QSVEESGGRLVTPGTPLTLT |
| | | CTVSGFSLS |
| 42 | Heavy FR2 | WVRQAPGKGLEWIG |
| 43 | Heavy FR3 | RFTISRTSTTVDLKIPSPTT |
| | | EDTATYFCAR |
| 44 | Heavy FR4 | WGPGTLVTVSL |
| 45 | Light FR1 | AVVMTQTASPVSAAVGGTVT |
| | | IKC |
| 46 | Light FR2 | WYQQKPGQPPKLLIA |
| 47 | Light FR3 | GVPSRFKGSGSGTEFTLTIS |
| | | DLECADAATYYC |
| 48 | Light FR4 | FGGGTEVVVE |

TABLE C
Sequence of antibody 27A2-1

| | | |
|---|---|---|
| SEQ ID | | |
| NO: | Description | Sequence |

27A2-1

| | | |
|---|---|---|
| 49 | VH domain | CAGTCGCTGGAGGAGGCTGG |
| | (nucleotide) | GGGTCGCCTGGTAACGCCTG |
| | | GAGGATCCCTGACACTCACC |
| | | TGCACAGCCTCTGGATTGTC |
| | | CCTCAGCAGCTACGACATGA |
| | | ACTGGGTCCGCCAGGCTCCA |
| | | GGGAAGGGGCTGGAATTCAT |
| | | CGGATTCATTAATACTCGTG |
| | | GTACCGCATACTACGCGAGC |
| | | TGGGCAAAAGGCCGATTCAC |
| | | CATCTCCAGAACCTCGACCA |
| | | CGGTGGATCTGACAATGACC |
| | | AGTCCGACAACCGAGGACAC |
| | | GGCCACCTATTTCTGTGCCA |
| | | GAGATATTCCTGATTGGAAT |
| | | ATTGGTCATGTTTTTGATCT |
| | | CTGGGGCCCAGGCACCCTGG |
| | | TCACCGTCTCCTCA |
| 50 | VL domain | GCCTATGATATGACCCAGAC |
| | (nucleotide) | TCCAGCCTCTGTGGAGGTAA |
| | | CTATGGGAGGCGCAGTCACC |
| | | ATCAATTGCCAGGCCAGTCA |
| | | GAGCATTACCAATGAATTAG |
| | | CCTGGTATCAGCAGAAACCA |
| | | GGGCAGCGTCCCAGGCTCCT |
| | | GATTTACCAGGCTTCTGCTC |
| | | TGGCATCTGGGGTCCCATCG |
| | | CGGTTCAAAGGCAGTGGATA |
| | | TGGGACACAGTTCACTCTCA |
| | | CCATCAGCGACCTGGAGTGT |
| | | GCCGATGCTGCCACTTACTA |
| | | CTGTCAACAGGGTTATACTA |
| | | GTAATAATATTGTCAATGCT |
| | | TTCGGCGGAGGGACCGAGGT |
| | | GGTGGTCAAA |
| 51 | VH domain (amino | QSLEEAGGRLVTPGGSLTLT |
| | acid) | CTASGLSLSSYDMNWVRQAP |
| | | GKGLEFIGFINTRGTAYYAS |
| | | WAKGRFTISRTSTTVDLTMT |
| | | SPTTEDTATYFCARDIPDWN |
| | | IGHVFDLWGPGTLVTVSS |
| 52 | VL domain (amino | AYDMTQTPASVEVTMGGAVT |
| | acid) | INCQASQSITNELAWYQQKP |
| | | GQRPRLLIYQASALASGVPS |
| | | RFKGSGYGTQFTLTISDLEC |
| | | ADAATYYCQQGYTSNNIVNA |
| | | FGGGTEVVVK |
| 53 | Heavy CDR1 | SYDMN |
| 54 | Heavy CDR2 | FINTRGTAYYASWAKG |
| 55 | Heavy CDR3 | DIPDWNIGHVFDL |
| 56 | Light CDR1 | QASQSITNELA |
| 57 | Light CDR2 | QASALAS |
| 58 | Light CDR3 | QQGYTSNNIVNA |
| 59 | Heavy FR1 | QSLEEAGGRLVTPGGSLTLT |
| | | CTASGLSLS |
| 60 | Heavy FR2 | WVRQAPGKGLEFIG |
| 61 | Heavy FR3 | RFTISRTSTTVDLTMTSPTT |
| | | EDTATYFCAR |
| 62 | Heavy FR4 | WGPGTLVTVSS |
| 63 | Light FR1 | AYDMTQTPASVEVTMGGAVT |
| | | INC |
| 64 | Light FR2 | WYQQKPGQRPRLLIY |
| 65 | Light FR3 | GVPSRFKGSGYGTQFTLTIS |
| | | DLECADAATYYC |
| 66 | Light FR4 | FGGGTEVVVK |

TABLE D
Sequences of antibody 29B5-1

| | | |
|---|---|---|
| SEQ ID | | |
| NO: | Description | Sequence |

29B5-1

| | | |
|---|---|---|
| 67 | VH domain | CAGTCGCTGGAGGAGTCCGG |
| | (nucleotide) | GGGTCGCCTGGTCACGCCTG |
| | | GGACACCCCTGACACTCACC |
| | | TGCACAGTCTCTGGAATCGA |
| | | CCTCAGTAGCGTGACAATGG |
| | | GCTGGGTCCGCCAGGCTCCA |
| | | GGGAAGGGGCTGGAATACAT |
| | | CGGAGTCATAACTACTATTC |
| | | GTACCACATACTACGCGAGC |
| | | TGGGTGAATGGTCGATTCAC |
| | | CATCTCCCAAACCTCGACCA |
| | | CGGTGGATCTGAAAATGACC |
| | | AGTCTGACAACCGAGGACAC |
| | | GGCCACCTATTTCTGTGCCA |
| | | GAGATGGTGATACTACTAAT |
| | | ACTTATGGCTACTACTTTGA |
| | | CATCTGGGGCCCAGGCACCC |
| | | TGGTCACCGTCTCCTTA |
| 68 | VL domain | GCCGTCGTGATGACCCAGAC |
| | (nucleotide) | TCCAGCCTCTGTGGAGGTAG |
| | | CTGTGGGAGGCACAGTCACC |
| | | ATTAAGTGCCAGGCCAGTCA |
| | | GAGCATTAGTAGCTACTTAG |
| | | CCTGGTATCAGCAGAAACCA |
| | | GGGCAGCCTCCCAAGCTCCT |
| | | GATCTACAAGGCTTCCACTC |
| | | TGGCATCTGGGGTCCCATCG |
| | | CGGTTCAAAGGCAGTGGATC |
| | | TGGGACACAGTTCACTCTCA |
| | | CCATCAGCGGCGTGCAGTGT |
| | | GACGATGCTGCCACTTACTA |
| | | CTGTCAACAGGGTTATACTG |
| | | GTAATAATGTTGATAATATT |
| | | TTCGGCGGAGGGACCGAGGT |
| | | GGTGGTCAAA |
| 69 | VH domain | QSLEESGGRLVTPGTPLTLT |
| | (amino | CTVSGIDLSSVTM |
| | acid) | GWVRQAPGKGLEYIGVITTI |
| | | RTTYYASWVNGRF |
| | | TISQTSTTVDLKMTSLTTED |
| | | TATYFCARDGDTTN |
| | | TYGYYFDIWGPGTLVTVSL |
| 70 | VL domain | AVVMTQTPASVEVAVGGTVT |
| | (amino | IKCQASQSISSYL |
| | acid) | AWYQQKPGQPPKLLIYKAST |
| | | LASGVPSRFKGSG |
| | | SGTQFTLTISGVQCDDAATY |
| | | YCQQGYTGNNVD |
| | | NIFGGGTEVVVK |
| 71 | Heavy CDR1 | SVTMG |
| 72 | Heavy CDR2 | VITTIRTTYYASWVNG |
| 73 | Heavy CDR3 | DGDTTNTYGYYFDI |
| 74 | Light CDR1 | QASQSISSYLA |
| or | | |
| 38 | | |
| 75 | Light CDR2 | KASTLAS |
| 76 | Light CDR3 | QQGYTGNNVDNI |
| 77 | Heavy FR1 | QSLEESGGRLVTPGTPLTLT |
| | | CTVSGIDLS |
| 78 | Heavy FR2 | WVRQAPGKGLEYIG |
| 79 | Heavy FR3 | RFTISQTSTTVDLKMTSLTT |
| | | EDTATYFCAR |
| 80 | Heavy FR4 | WGPGTLVTVSL |
| 81 | Light FR1 | AVVMTQTPASVEVAVGGTVT |
| | | IKC |
| 82 | Light FR2 | WYQQKPGQPPKLLIY |
| 83 | Light FR3 | GVPSRFKGSGSGTQFTLTIS |
| | | GVQCDDAATYYC |
| 84 | Light FR4 | FGGGTEVVVK |

TABLE E
Sequences of 2B11-1

| | | |
|---|---|---|
| SEQ ID | | |
| NO: | Description | Sequence |

2B11-1

| | | |
|---|---|---|
| 85 | VH domain | CAGTCGGTGGGGGAGTCCGG |
| | (nucleotide) | GGGTCGCCTGGTCACGCCTG |
| | | GGACACCCCTGACACTCACC |
| | | TGCACAGTCTCTGGAATCGA |
| | | CCTCAGTATCTATCCAGTAG |
| | | ACTGGGTCCGCCAGGCTCCA |
| | | GGGAAGGGGCTGGAGTGGAT |
| | | CGGAATCATTAATACCAATG |
| | | GTAACACATACTACGCGAAC |
| | | TGGGCGAAAGGCCGATTCAC |
| | | CATCTCCAAAACCTCGTCGA |
| | | CCACGGTGGATCTGCAAGTG |
| | | ACCAGTCTGACAACCGAGGA |
| | | CACGGCCACCTATTTCTGTG |
| | | CCAGACACTTTGACTTGTGG |
| | | GGCCAAGGCACCCTGGTCAC |
| | | CGTCTCCTCA |
| 86 | VL domain | CAAGTGCTGACCCAGACTGC |
| | (nucleotide) | ATCCCCCGTGTCTGCGGCTG |
| | | TTGGAGGCACAGTCACCATC |
| | | AATTGCCAGTCCAGTCAGAC |
| | | TGTTTATAATAACAACAACT |
| | | TAGCCTGGTATCAGCAGAAA |
| | | TCAGGGCAGCCTCCCAAGCT |
| | | CCTGATCTATGATTCATCCA |
| | | AACTGGAATCTGGGGTCCCA |
| | | TCGCGGTTCAGCGGCAGTGG |
| | | ATCTGGGACACAGTTCACTC |
| | | TCACCATCAGCGACCTGGAG |
| | | TGTGACGATGCTGCCACTTA |
| | | CTATTGTGCAGGCGGTTATG |
| | | GTGGTTGGATTGCTGCTTTC |
| | | GGCGGAGGGACCGAGGTGGT |
| | | GGTCAAA |
| 87 | VH domain (amino | QSVGESGGRLVTPGTPLTLT |
| | acid) | CTVSGIDLSIYPVDWVRQAP |
| | | GKGLEWIGIINTNGNTYYAN |
| | | WAKGRFTISKTSSTTVDLQV |
| | | TSLTTEDTATYFCARHFDLW |
| | | GQGTLVTVSS |
| 88 | VL domain (amino | QVLTQTASPVSAAVGGTVTI |
| | acid) | NCQSSQTVYNNNNLAWYQQK |
| | | SGQPPKLLIYDSSKLESGVP |
| | | SRFSGSGSGTQFTLTISDLE |
| | | CDDAATYYCAGGYGGWIAAF |
| | | GGGTEVVVK |
| 89 | Heavy CDR1 | IYPVD |
| 90 | Heavy CDR2 | IINTNGNTYYANWAKG |
| 91 | Heavy CDR3 | HFDL |
| 92 | Light CDR1 | QSSQTVYNNNNLA |
| 93 | Light CDR2 | DSSKLES |
| or | | |
| 21 | | |
| 94 | Light CDR3 | AGGYGGWIAA |
| 95 | Heavy FR1 | QSVGESGGRLVTPGTPLTLT |
| | | CTVSGIDLS |
| 96 | Heavy FR2 | WVRQAPGKGLEWIG |
| 97 | Heavy FR3 | RFTISKTSSTTVDLQVTSLT |
| | | TEDTATYFCAR |
| 98 | Heavy FR4 | WGQGTLVTVSS |
| 99 | Light FR1 | QVLTQTASPVSAAVGGTVTI |
| | | NC |
| 100 | Light FR2 | WYQQKSGQPPKLLIY |
| 101 | Light FR3 | GVPSRFSGSGSGTQFTLTIS |
| | | DLECDDAATYYC |
| 102 | Light FR4 | FGGGTEVVVK |

TABLE F
Sequences of antibody 33B6-1

| | | |
|---|---|---|
| SEQ ID | | |
| NO: | Description | Sequence |

33B6-1

| | | |
|---|---|---|
| 103 | VH domain | CAGTCGCTGGAGGAGTCCGG |
| | (nucleotide) | GGGTCGCCTGGTCACGCCTG |
| | | GGACACCCCTGACACTCACC |
| | | TGCACAGCCTCTGGATTGTC |
| | | CCTCAGCAGCTACGACATGA |
| | | ACTGGGTCCGCCAGGCTCCA |
| | | GGGAAGGGGCTGGAATACAT |
| | | CGGATTCATTAATACTCGTG |
| | | GTAGCGCATACTACGCGAGC |
| | | TGGGCTACAGGCCGATTCAC |
| | | CATCTCCAGAACCTCGACCA |
| | | CGGTGGATCTGACAATGACC |
| | | AGTCCGACAACCGAGGACAC |
| | | GGCCACCTATTTCTGTGCCA |
| | | GAGATATTCCTGATTGGGAT |
| | | ATTGGTCATGTTTTTGATCT |
| | | CTGGGGCCCAGGCACCCTGG |
| | | TCACCGTCTCCTCA |
| 104 | VL domain | GCCTATGATATGACCCAGAC |
| | (nucleotide) | TCCAGCCTCTGTGGAGGTAA |
| | | CTATGGGAGGCGCAGTCACC |
| | | ATCAAGTGCCAGGCCAGTCA |
| | | GAGCATTAGCAATGAATTAT |
| | | CCTGGTATCAACAGAAACCA |
| | | GGGCAGCGTCCCAGGCTCCT |
| | | GATTTACCAGGCTTCTGCTC |
| | | TGGCATCTGGGGTCCCATCG |
| | | CGGTTCAAAGGCAGTGGATA |
| | | TGGGACACAGTTCACTCTCA |
| | | CCATCAGCGACCTGGAGTGT |
| | | GCCGATGCTGCCACTTACTA |
| | | CTGTCAACAGGGTTATACTA |
| | | GTAATAATATTGTCAATGCT |
| | | TTCGGGGGAGGGACCGAGGT |
| | | GGTGGTCAAA |
| 105 | VH domain (amino | QSLEESGGRLVTPGTPLTLT |
| | acid) | CTASGLSLSSYDMNWVRQAP |
| | | GKGLEYIGFINTRGSAYYAS |
| | | WATGRFTISRTSTTVDLTMT |
| | | SPTTEDTATYFCARDIPDWD |
| | | IGHVFDLWGPGTLVTVSS |
| 106 | VL domain (amino | AYDMTQTPASVEVTMGGAVT |
| | acid) | IKCQASQSISNELSWYQQKP |
| | | GQRPRLLIYQASALASGVPS |
| | | RFKGSGYGTQFTLTISDLEC |
| | | ADAATYYCQQGYTSNNIVNA |
| | | FGGGTEVVVK |
| 107 or | Heavy CDR1 | SYDMN |
| 53 | | |
| 108 | Heavy CDR2 | FINTRGSAYYASWATG |
| 109 | Heavy CDR3 | DIPDWDIGHVFDL |
| 110 | Light CDR1 | QASQSISNELS |
| 111 or | Light CDR2 | QASALAS |
| 57 | | |
| 112 or | Light CDR3 | QQGYTSNNIVNA |
| 58 | | |
| 113 | Heavy FR1 | QSLEESGGRLVTPGTPLTLT |
| | | CTASGLSLS |
| 114 | Heavy FR2 | WVRQAPGKGLEYIG |
| 115 | Heavy FR3 | RFTISRTSTTVDLTMTSPTT |
| | | EDTATYFCAR |
| 116 | Heavy FR4 | WGPGTLVTVSS |
| 117 | Light FR1 | AYDMTQTPASVEVTMGGAVT |
| | | IKC |
| 118 | Light FR2 | WYQQKPGQRPRLLIY |
| 119 | Light FR3 | GVPSRFKGSGYGTQFTLTIS |
| | | DLECADAATYYC |
| 120 | Light FR4 | FGGGTEVVVK |

TABLE G
Sequences of antibody 4D11-1

| | | |
|---|---|---|
| SEQ ID | | |
| NO: | Description | Sequence |

4D11-1

| | | |
|---|---|---|
| 121 | VH domain | CAGGAGCAAGTGGTGGAGTC |
| | (nucleotide) | CGGGGGAGGCCTGGTCCAGC |
| | | CTGAGGGATCCCTGACACTC |
| | | ACCTGCACAGCCTCTGGATT |
| | | CGACTTCAGTAGCAATGCAA |
| | | TGTGCTGGGTCCGCCAGGCT |
| | | CCAGGGAAGGGGCTGGCGTG |
| | | GATCGGATGCATTGGTACTG |
| | | GTAGTAGTGGTAGCACTTAC |
| | | TACGCGAGCTGGGCGAGAGG |
| | | CCGATTCACCATCTCCAAAA |
| | | CCTCGTCGACCACGGTGACT |
| | | CTGCAAATGACCAGTCTGAC |
| | | AGCCGCGGACACGGCCACCT |
| | | ATTTCTGTCCGAGAGACATT |
| | | AATGCTTATGGTAATGATGG |
| | | TTGGTCTTTTAACTTGTGGG |
| | | GCCCAGGCACCCTGGTCACC |
| | | GTCTCGTCA |
| 122 | VL domain | GCCTATGATATGACCCAGAC |
| | (nucleotide) | TCCAGCCTCTGTGGAGGTAG |
| | | CTGTGGGAGGCACAGTCACC |
| | | ATCAAGTGCCAGGCCAGTCA |
| | | GAGTATTGTTGGTTGGTTAT |
| | | CCTGGTATCAGCAGAAACCC |
| | | GGGCAGCCTCCCAAGCTCCT |
| | | GATCTACAGGGCATCCACTC |
| | | TGGCATCTGGGGTCCCATCG |
| | | CGGTTCAAAGGCAGTGGATC |
| | | TGGGACAGAGTTCACTCTCA |
| | | CCATCAGCGACCTGGAGTGT |
| | | GACGATGCTGCCACTTACTA |
| | | CTGTCAACAGGGTTATACTT |
| | | TTAATAATGTTGATAATCCT |
| | | TTCGGCGGAGGGACCGAGGT |
| | | GTTGGTCAAA |
| 123 | VH domain | QEQVVESGGGLVQPEGSLTL |
| | (amino | TCTASGFDFSSNAMCWVRQA |
| | acid) | PGKGLAWIGCIGTGSSGSTY |
| | | YASWARGRFTISKTSSTTVT |
| | | LQMTSLTAADTATYFCPRDI |
| | | NAYGNDGWSFNLWGPGTLVT |
| | | VSS |
| 124 | VL domain | AYDMTQTPASVEVAVGGTVT |
| | (amino | IKCQASQSIVGWLSWYQQKP |
| | acid) | GQPPKLLIYRASTLASGVPS |
| | | RFKGSGSGTEFTLTISDLEC |
| | | DDAATYYCQQGYTFNNVDNP |
| | | FGGGTEVLVK |
| 125 | Heavy CDR1 | SNAMC |
| 126 | Heavy CDR2 | CIGTGSSGSTYYASWARG |
| 127 | Heavy CDR3 | DINAYGNDGWSFNL |
| 128 | Light CDR1 | QASQSIVGWLS |
| 129 | Light CDR2 | RASTLAS |
| 130 | Light CDR3 | QQGYTFNNVDNP |
| 131 | Heavy FR1 | QEQVVESGGGLVQPEGSLTL |
| | | TCTASGFDFS |
| 132 | Heavy FR2 | WVRQAPGKGLAWIG |
| 133 | Heavy FR3 | RFTISKTSSTTVTLQMTSLT |
| | | AADTATYFCPR |
| 134 | Heavy FR4 | WGPGTLVTVSS |
| 135 | Light FR1 | AYDMTQTPASVEVAVGGTVT |
| | | IKC |
| 136 | Light FR2 | WYQQKPGQPPKLLIY |
| 137 | Light FR3 | GVPSRFKGSGSGTEFTLTIS |
| | | DLECDDAATYYC |
| 138 | Light FR4 | FGGGTEVLVK |

TABLE H
Sequences of antibody 54F1-1

| | | |
|---|---|---|
| SEQ ID | | |
| NO: | Description | Sequence |

54F1-1

| | | |
|---|---|---|
| 139 | VH domain | CAGTCGGTGGGGGAGTCCGG |
| | (nucleotide) | GGGTCGCCTGGTCACGCCTG |
| | | GGACACCCCTGACACTCACC |
| | | TGCACAGTCTCTGGAATCGA |
| | | CCTCAGCGTCTATCCAGTAG |
| | | ACTGGGTCCGCCAGGCTCCA |
| | | GGGAAGGGGCTGGAGTGGAT |
| | | CGGGATCATTAGTACCAATG |
| | | GGAACACATACTACGCGAAC |
| | | TGGGCGAAGGGCCGATTCAC |
| | | CATCTCCAAAACCTCGTCGA |
| | | CCACGGTGGATCTGAAAATG |
| | | ACCAGTCTGACAACCGAGGA |
| | | CACGGCCACCTATTTCTGTG |
| | | CCAGACACTTTGACTTGTGG |
| | | GGCCAAGGCACTCTGGTCAC |
| | | CGTCTCCTCA |
| 140 | VL domain | CAAGTGCTGACCCAGACTGC |
| | (nucleotide) | ATCCCCCGTGTCTGCGGCTG |
| | | TGGGAGGCACAGTCACCATC |
| | | AATTGCCAGTCCAGTCAGAC |
| | | TGTTTATAATAACAATAACT |
| | | TAGCCTGGTATCAGCAGAAA |
| | | TCAGGGCAGCCTCCCAAGCT |
| | | CCTGATTTATGATTCATTCA |
| | | AACTGGAATCTGGGGTCCCA |
| | | TCGCGGTTCAGCGGCAGTGG |
| | | ATCTGGGACACAGTTCACTC |
| | | TCACCATCAGCGACCTGGAG |
| | | TGTGACGATGCTGCCACTTA |
| | | CTATTGTGCAGGCGGTTATG |
| | | CTGGTTGGATTGCTGCTTTC |
| | | GGCGGAGGGACCGAGGTGGT |
| | | GGTCAAA |
| 141 | VH domain | QSVGESGGRLVTPGTPLTLT |
| | (amino | CTVSGIDLSVYPVDWVRQAP |
| | acid) | GKGLEWIGIISTNGNTYYAN |
| | | WAKGRFTISKTSSTTVDLKM |
| | | TSLTTEDTATYFCARHFDLW |
| | | GQGTLVTVSS |
| 142 | VL domain | QVLTQTASPVSAAVGGTVTI |
| | (amino | NCQSSQTVYNNNN |
| | acid) | LAWYQQKSGQPPKLLIYDSF |
| | | KLESGVPSRFSGS |
| | | GSGTQFTLTISDLECDDAAT |
| | | YYCAGGYAGWIAA |
| | | FGGGTEVVVK |
| 143 | Heavy CDR1 | VYPVD |
| 144 | Heavy CDR2 | IISTNGNTYYANWAKG |
| 145 or | Heavy CDR3 | HFDL |
| 91 | | |
| 146 or | Light CDR1 | QSSQTVYNNNNLA |
| 92 | | |
| 147 | Light CDR2 | DSFKLES |
| 148 | Light CDR3 | AGGYAGWIAA |
| 149 | Heavy FR1 | QSVGESGGRLVTPGTPLTLT |
| | | CTVSGIDLS |
| 150 | Heavy FR2 | WVRQAPGKGLEWIG |
| 151 | Heavy FR3 | RFTISKTSSTTVDLKMTSLT |
| | | TEDTATYFCAR |
| 152 | Heavy FR4 | WGQGTLVTVSS |
| 153 | Light FR1 | QVLTQTASPVSAAVGGTVTI |
| | | NC |
| 154 | Light FR2 | WYQQKSGQPPKLLIY |
| 155 | Light FR3 | GVPSRFSGSGSGTQFTLTIS |
| | | DLECDDAATYYC |
| 156 | Light FR4 | FGGGTEVVVK |

TABLE I
Sequences of antibody 69H7-1

| | | |
|---|---|---|
| SEQ ID | | |
| NO: | Description | Sequence |

69H7-1

| | | |
|---|---|---|
| 157 | VH domain | CAGTCGCTGGAGGAGTCCGG |
| | (nucleotide) | GGGTCGCCTGGTCACGCCTG |
| | | GGACACCCCTGACACTCACC |
| | | TGCACAGCCTCTGGATTGTC |
| | | CCTCAGCAGGTACGACATGA |
| | | ACTGGGTCCGCCAGGCTCCA |
| | | GGGAAGGGGCTGGAATACAT |
| | | CGGATTCATTAATACTCGTG |
| | | GTAGCACATACTACGCGACC |
| | | TGGACTACAGGCCGATTCAC |
| | | CATCTCCAGAACCTCGACCA |
| | | CGGTGGATCTGACAATGACC |
| | | AGTCCGACAACCGAGGACAC |
| | | GGCCACCTATTTCTGTGCCA |
| | | GAGATATTCCTGATTGGGAT |
| | | ATTGGTCATGTTTTTGATCT |
| | | CTGGGGCCCAGGCACCCTGG |
| | | TCACCGTCTCCTCA |
| 158 | VL domain | GCCTATGATATGACCCAGAC |
| | (nucleotide) | TCCAGCCTCTGTGTCTGGAG |
| | | CTGTGGGAGGCACAGTCACC |
| | | ATCAATTGCCAGGCCAGTCA |
| | | GAGCATTAGCAATGAATTAT |
| | | CCTGGTATCAACAGAAACCA |
| | | GGGCAGCGTCCCAGGCTCCT |
| | | GATTTACCAGGCTTCTACTC |
| | | TGGCATCTGGGGTCCCATCG |
| | | CGGTTCAAAGGCAGTGGATA |
| | | TGGGACACAGTTCACTCTCA |
| | | CCATCAGCGACCTGGAGTGT |
| | | GCCGATGCTGCCACTTACTA |
| | | CTGTCAACAGGGTTATACTA |
| | | GTAATAATATTGTCAATGCT |
| | | TTCGGCGGAGGGACCGAGGT |
| | | GGTGGTCAAA |
| 159 | VH domain | QSLEESGGRLVTPGTPLTLT |
| | (amino | CTASGLSLSRYDMNWVRQAP |
| | acid) | GKGLEYIGFINTRGSTYYAT |
| | | WTTGRFTISRTSTTVDLTMT |
| | | SPTTEDTATYFCARDIPDWD |
| | | IGHVFDLWGPGTLVTVSS |
| 160 | VL domain | AYDMTQTPASVSGAVGGTVT |
| | (amino | INCQASQSISNELSWYQQKP |
| | acid) | GQRPRLLIYQASTLASGVPS |
| | | RFKGSGYGTQFTLTISDLEC |
| | | ADAATYYCQQGYTSNNIVNA |
| | | FGGGTEVVVK |
| 161 | Heavy CDR1 | RYDMN |
| 162 | Heavy CDR2 | FINTRGSTYYATWTTG |
| 163 or | Heavy CDR3 | DIPDWDIGHVFDL |
| 109 | | |
| 164 or | Light CDR1 | QASQSISNELS |
| 110 | | |
| 165 | Light CDR2 | QASTLAS |
| 166 | Light CDR3 | QQGYTSNNIVNA |
| or | | |
| 58 | | |
| or | | |
| 112 | | |
| 167 | Heavy FR1 | QSLEESGGRLVTPGTPLTLT |
| | | CTASGLSLS |
| 168 | Heavy FR2 | WVRQAPGKGLEYIG |
| 169 | Heavy FR3 | RFTISRTSTTVDLTMTSPTT |
| | | EDTATYFCAR |
| 170 | Heavy FR4 | WGPGTLVTVSS |
| 171 | Light FR1 | AYDMTQTPASVSGAVGGTVT |
| | | INC |
| 172 | Light FR2 | WYQQKPGQRPRLLIY |
| 173 | Light FR3 | GVPSRFKGSGYGTQFTLTIS |
| | | DLECADAATYYC |
| 174 | Light FR4 | FGGGTEVVVK |

TABLE J
Sequences of antibody 75C9-1

| | | |
|---|---|---|
| SEQ ID | | |
| NO: | Description | Sequence |

75C9-1

| | | |
|---|---|---|
| 175 | VH domain | CAGGAGCAGGTGGTGGAGTC |
| | (nucleotide) | CGGGGGAGGCCTGGTCCAGC |
| | | CTGAGGGATCCCTGACACTC |
| | | ACCTGCACAGCTTCTGGATT |
| | | CTCCTTTAATAATAATGCAA |
| | | TGTGCTGGGTCCGCCAGGCT |
| | | CCAGGGAAGGGGCTGGAGTG |
| | | GATCGGATGCATTGGTACTG |
| | | CTGCTAGTGGTAATAGTTAC |
| | | TACGCGAGCTGGGCGAAAGG |
| | | CAGATTTACCATCTCCAGAA |
| | | CCTCGTCGACCACGGTGACG |
| | | CTACAAATGACCAGTCTGAC |
| | | AGCCGCGGACACGGCCACCT |
| | | ATTTCTGTCCGAGAGACATT |
| | | AATGCTTATGGTAATGATGG |
| | | TTGGTCTTTTAAGTTGTGGG |
| | | GCCCAGGCACCCTGGTCACC |
| | | GTCTCCTCA |
| 176 | VL domain | GCATTCGAATTGACCCAGAC |
| | (nucleotide) | TCCATCCTCCGTGGAGGCAG |
| | | CTGTGGGAGGCACAGTCACC |
| | | ATCAAGTGCCAGGCCAGTCA |
| | | GAGCATTAATGGTTGGTTAT |
| | | CCTGGTATCAGCAGAAACCA |
| | | GGGCAGCCTCCCAAGCTCCT |
| | | GATCTATAGGGCATCGCCTG |
| | | TGGCATCTGGGGTCCCATCG |
| | | CGGTTCAAAGGCAGTGGATC |
| | | TGGGACAGAGTACACTCTCA |
| | | CCATCAGCGGCGTGCAGTGT |
| | | GACGATGCTGCCACTTACTA |
| | | CTGTCAACAGGGTTATACTT |
| | | TTAATAATGTTGACGATCCT |
| | | TTCGGCGGAGGGACCGAGGT |
| | | GGTGGTCAAA |
| 177 | VH domain | QEQVVESGGGLVQPEGSLTL |
| | (amino | TCTASGFSFNNNAMCWVRQA |
| | acid) | PGKGLEWIGCIGTAASGNSY |
| | | YASWAKGRFTISRTSSTTVT |
| | | LQMTSLTAADTATYFCPRDI |
| | | NAYGNDGWSFKLWGPGTLVT |
| | | VSS |
| 178 | VL domain | AFELTQTPSSVEAAVGGTVT |
| | (amino | IKCQASQSINGWLSWYQQKP |
| | acid) | GQPPKLLIYRASPVASGVPS |
| | | RFKGSGSGTEYTLTISGVQC |
| | | DDAATYYCQQGYTFNNVDDP |
| | | FGGGTEVVVK |
| 179 | Heavy CDR1 | NNAMC |
| 180 | Heavy CDR2 | CIGTAASGNSYYASWAKG |
| 181 | Heavy CDR3 | DINAYGNDGWSFKL |
| 182 | Light CDR1 | QASQSINGWLS |
| 183 | Light CDR2 | RASPVAS |
| 184 | Light CDR3 | QQGYTFNNVDDP |
| 185 | Heavy FR1 | QEQVVESGGGLVQPEGSLTL |
| | | TCTASGFSFN |
| 186 | Heavy FR2 | WVRQAPGKGLEWIG |
| 187 | Heavy FR3 | RFTISRTSSTTVTLQMTSLT |
| | | AADTATYFCPR |
| 188 | Heavy FR4 | WGPGTLVTVSS |
| 189 | Light FR1 | AFELTQTPSSVEAAVGGTVT |
| | | IKC |
| 190 | Light FR2 | WYQQKPGQPPKLLIY |
| 191 | Light FR3 | GVPSRFKGSGSGTEYTLTIS |
| | | GVQCDDAATYYC |
| 192 | Light FR4 | FGGGTEVVVK |

TABLE K
Sequences of antibody 9E4-1

| | | |
|---|---|---|
| SEQ ID | | |
| NO: | Description | Sequence |

9E4-1

| | | |
|---|---|---|
| 193 | VH domain | CAGTCGCTGGAGGAGTCCGG |
| | (nucleotide) | GGGTCGCCTGGTCACGCCTG |
| | | GGACACCCCTGACACTCACC |
| | | TGCACAGTCTCTGGAATCGA |
| | | CCTCAGTAGCGTGACAATGG |
| | | GCTGGGTCCGCCAGGCTCCA |
| | | GGGAAGGGGCTGGAATACAT |
| | | CGGAGTCATTGTTCCTCGTG |
| | | GTGCCACATACTACGCGAAC |
| | | TGGGTGAAAGGCCGATTCAC |
| | | CATCTCCAAAACCTCGACCA |
| | | CGGTGGATCTGAAAATGACC |
| | | AGTCTGACAACCGAGGACAC |
| | | GGCCACCTATTTCTGTGCCA |
| | | GAGATGGTGATACTGGTAAT |
| | | ATTTATGGCTATTATTTTGA |
| | | CATCTGGGGCCCAGGCACCC |
| | | TGGTCACCGTCTCCTTA |
| 194 | VL domain | GCCTATGATATGACCCAGAC |
| | (nucleotide) | TCCAGCCTCCGTGTCTGAAC |
| | | CTGTGGGAGGCACAGTCACC |
| | | ATTAAGTGCCAGGCCAGTCA |
| | | GAGCATTAGCACTGCATTAG |
| | | CCTGGTATCAGCAGAAATCA |
| | | GGGCAGCGTCCCAAGCTCCT |
| | | GATCTACGATGCATGGAAAC |
| | | TGGCATCTGGGGTCTCATCG |
| | | CGGTTCAAAGGCAGTGGATC |
| | | TGGGACACAGTTCACTCTCA |
| | | CCATCAGTGGCGTGCAGTGT |
| | | GACGATGCTGCCACTTACTA |
| | | CTGTCAACAGGGTTATAGTG |
| | | GTAATAATGTTGATAATGTT |
| | | TTCGGCGGAGGGACCGAGGT |
| | | GGTGGTCAGC |
| 195 | VH domain | QSLEESGGRLVTPGTPLTLT |
| | (amino | CTVSGIDLSSVTMGWVRQAP |
| | acid) | GKGLEYIGVIVPRGATYYAN |
| | | WVKGRFTISKTSTTVDLKMT |
| | | SLTTEDTATYFCARDGDTGN |
| | | IYGYYFDIWGPGTLVTVSL |
| 196 | VL domain | AYDMTQTPASVSEPVGGTVT |
| | (amino | IKCQASQSISTALAWYQQKS |
| | acid) | GQRPKLLIYDAWKLASGVSS |
| | | RFKGSGSGTQFTLTISGVQC |
| | | DDAATYYCQQGYSGNNVDNV |
| | | FGGGTEVVVS |
| 197 or | Heavy CDR1 | SVTMG |
| 71 | | |
| 198 | Heavy CDR2 | VIVPRGATYYANWVKG |
| 199 | Heavy CDR3 | DGDTGNIYGYYFDI |
| 200 | Light CDR1 | QASQSISTALA |
| 201 | Light CDR2 | DAWKLAS |
| 202 | Light CDR3 | QQGYSGNNVDNV |
| 203 | Heavy FR1 | QSLEESGGRLVTPGTPLTLT |
| | | CTVSGIDLS |
| 204 | Heavy FR2 | WVRQAPGKGLEYIG |
| 205 | Heavy FR3 | RFTISKTSTTVDLKMTSLTT |
| | | EDTATYFCAR |
| 206 | Heavy FR4 | WGPGTLVTVSL |
| 207 | Light FR1 | AYDMTQTPASVSEPVGGTVT |
| | | IKC |
| 208 | Light FR2 | WYQQKSGQRPKLLIY |
| 209 | Light FR3 | GVSSRFKGSGSGTQFTLTIS |
| | | GVQCDDAATYYC |
| 210 | Light FR4 | FGGGTEVVVS |

TABLE L
Consensus amino acid sequences

| SEQ ID | | |
|---|---|---|
| NO: | Description | Sequence |
| 211 | Light CDR1 | Q A S Q S I X₇ X₈ X₉ L X₁₁ |
| | | wherein X₇, X₈, X₉ and |
| | | X₁₁ are any amino acid. |
| 212 | Light CDR1 | Q AS Q SI X₇ X₈ X₉ L X₁₁ |
| | | wherein X₇ is V or S or |
| | | T or N; X₈ is G or |
| | | N or S or T; |
| | | X₉ is W or E or |
| | | Y or A; X₁₁ is S or A |

The VL (i.e. light) CDR1 amino acid sequences of the 33B6-1, 69H7-1, 24C6-1, 29B5-1, 9E4-1, 27A2-1, 4D11-1 and 75C9-1 antibodies of the invention each fall within the consensus VL (i.e. light) CDR1 sequences of the above Table L.

TABLE M
Consensus amino acid sequences

| SEQ | | |
|---|---|---|
| ID | | |
| NO: | Description | Sequence |
| 213 | Light CDR1 | Q S S Q X₅ V Y N N N N L A |
| | | wherein X₅ is any amino acid. |
| 214 | Light CDR1 | Q SS Q X₅ VYNN NN LA |
| | | wherein X₅ is T or S. |

The VL (i.e. light) CDR1 amino acid sequences of the 2B₁₁-1, 54F1-1 and 23B9-1 antibodies of the invention each fall within the consensus VL (i.e. light) CDR1 sequences of the above Table M.

TABLE N
Consensus amino acid sequences

| SEQ | | |
|---|---|---|
| ID | | |
| NO: | Description | Sequence |
| 215 | Light CDR3 | Q Q G Y X₅ X₆ N N X₉ |
| | | X₁₀ X₁₁ X₁₂ |
| | | wherein |
| | | X₅, X₆, X₉, X₁₀, X₁₁ |
| | | and X₁₂ are any amino |
| | | acid. |
| 216 | Light CDR3 | Q Q G Y X₅ X₆ N N X₉ X₁₀ |
| | | X₁₁ X₁₂ |
| | | wherein X₅ is T or S; X₆ |
| | | is F or G or S; X₉ is V |
| | | or I X₁₀ is D or V; |
| | | X₁₁ is N or D; |
| | | X₁₂ is P or I or V or A. |

The VL (i.e. light) CDR3 amino acid sequences of the 33B6-1, 69H7-1, 29B5-1, 9E4-1, 27A2-1, 4D11-1 and 75C9-1 antibodies of the invention each fall within the consensus VL (i.e. light) CDR3 sequences of the above Table N.

TABLE O
Consensus amino acid sequences

| SEQ | | |
|---|---|---|
| ID | | |
| NO: | Description | Sequence |
| 217 | Light CDR3 | A G G Y X₅ G X₇ I A X₁₀ |
| | | wherein X₅, X₇ and |
| | | X₁₀ are any amino acid. |
| 218 | Light CDR3 | A G G Y X₅ G X₇ I A X₁₀ |
| | | wherein X₅ is G or A or S; |
| | | X₇ is W or Vand X₁₀ is A or |
| | | S. |

The VL (i.e. light) CDR3 amino acid sequences of the 2B₁₁-1, 54F1-1 and 23B9-1 antibodies of the invention each fall within the consensus VL (i.e. light) CDR3 sequences of the above Table O.

The invention will now be further described in the following non-limiting Examples with reference to the following drawings:

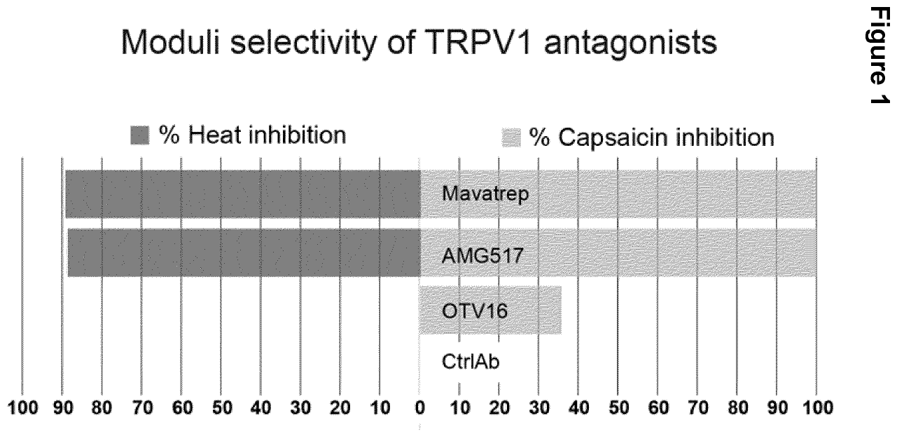

FIG. 1: Inhibition of heat activated or capsaicin activated TRPV1-activity after treatment with the OTV16 polyclonal antibody (cap n=7, heat n=6), AMG 517 (cap n=5, heat n=4), Mavatrep (cap n=3, heat n=3) or control antibody (cap n=4, heat n=7). The control antibody was Rabbit Gamma Globulin (RRID: AB_2532177—Thermofisher, catalogue number 31887). Mavatrep and AMG517 were evaluated at equal concentrations for analysing the inhibition of heat and capsaicin activation of TRPV1, whereas the inhibition of heat activation of TRPV1 with the antibodies was evaluated at 5× the concentration of the antibodies used for the evaluation of the inhibition of capsaicin activation of TRPV1. Inhibition of capsaicin activation was evaluated with patch-clamp experiments and inhibition of heat activation was evaluated with fluorescence intensity recordings of heat induced TRPV1-mediated calcium uptake.

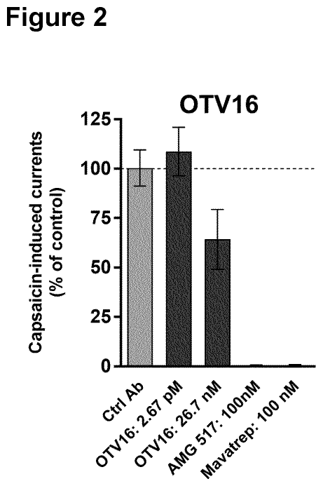

FIG. 2: Patch clamp recordings of capsaicin induced TRPV1-currents after treatment with the OTV16 polyclonal antibody, AMG 517, Mavatrep or control antibody. The control antibody was Rabbit Gamma Globulin (RRID: AB_2532177—Thermofisher, catalogue number 31887). The current amplitude for activation with capsaicin in the presence of antibody, calculated as a percentage of the amplitude for activation with capsaicin only, after treatment with either 2.67 pM OTV16 antibody (n=6), 26.7 nM OTV16 antibody (n=7), 100 nM Mavatrep (n=5), 100 nM AMG 517 (n=3) or 730 nM control antibody (n=4) is presented. Each data point (n) represents a single cell. Antibody treatment (OTV16) was compared to treatment with control antibody. Data is presented as mean±SEM.

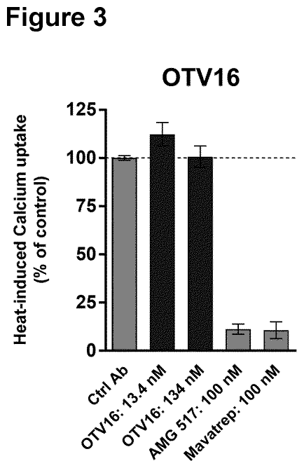

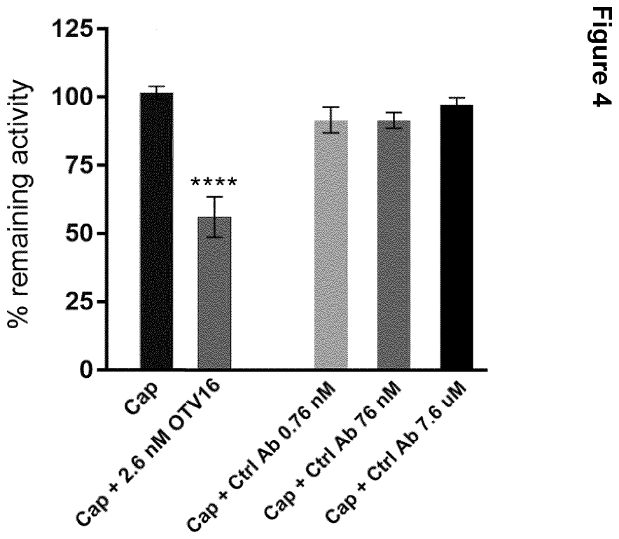

FIG. 3: Fluorescence intensity recordings of heat induced TRPV1-mediated calcium uptake after treatment with the OTV16 polyclonal antibody, AMG 517, Mavatrep or control antibody. The control antibody was Rabbit Gamma Globulin (RRID: AB_2532177—Thermofisher, catalogue number 31887). Antibody solutions was delivered using the Biopen® and heating to 42° C. was achieved using a heat-probe. Two pulses of heat were applied, the second in the presence of antibody. The fluorescence intensity for the second activation with heat in the presence of antibody, calculated as a percentage of the amplitude for the first activation with heat only, after treatment with either 13.4 nM OTV16 antibody (n=5), 134 nM OTV16 antibody (n=6), 100 nM Mavatrep (n=3), 100 nM AMG-517 (n=4) or 37 μM control antibody (n=7) is presented. Each data point (n) equals the number of experiments, each containing one or more cells. Antibody treatment (OTV16) was compared to treatment with control antibody. Data is presented as mean±SEM.

FIG. 4: Inhibition of capsaicin-induced calcium uptake for hTRPV1. CHO cells expressing hTRPV1 were incubated with the calcium indicator Fluo-3 AM for 30 min at 37° C. followed by antibody (OTV16 n=12 or control antibody n=12 (ThermoFischer #31887)) for 1 h, at room temperature. Calcium content within the cells was then monitored using a plate reader before and after application of 1 μM capsaicin and 150 μM Ca²⁺ to the antibody solution covering the cells. The total calcium uptake for each antibody was normalized against the calcium uptake for capsaicin activation only (i.e. capsaicin+calcium; “Cap” in FIG. 4). % remaining activity means the amount of activity remaining as compared to the 1 μM capsaicin and 150 μM Ca²⁺ reference (a control without antibody) which is labeled “Cap” on FIG. 4. Purely by way of an example, 30% remaining activity would mean that the activity is inhibited by 70%. Data is represented as mean±SEM. Statistical significance was determined using a Kruskal-Wallis test followed by Dunn's multiple comparison. Four asterisks=p value of less than 0.0001.

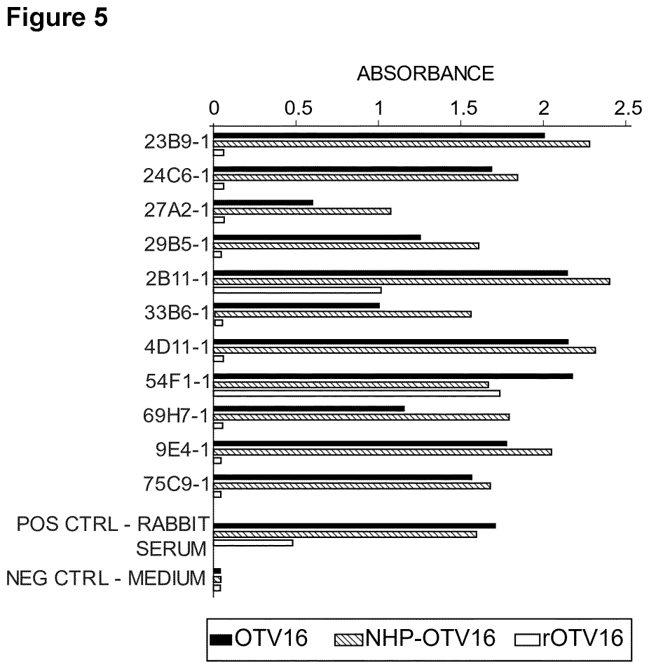

FIG. 5: Peptide ELISA of B-cell clone supernatants. Antibody in supernatant from B-cell clones was tested for binding to OTV16 peptide, NHP-OTV16 peptide and rOTV16 peptide respectively. B-cell supernatants were incubated for 1 hr in 96-well plates coated with the respective peptide. Plates were washed and bound antibody was detected using anti-rabbit IgG conjugated to horse radish peroxidase with absorbance measured at 450 nm. Serum from immunized rabbit was used as a positive binding control. B-cell culture medium (antibody-free) was used as a negative control. N=1.

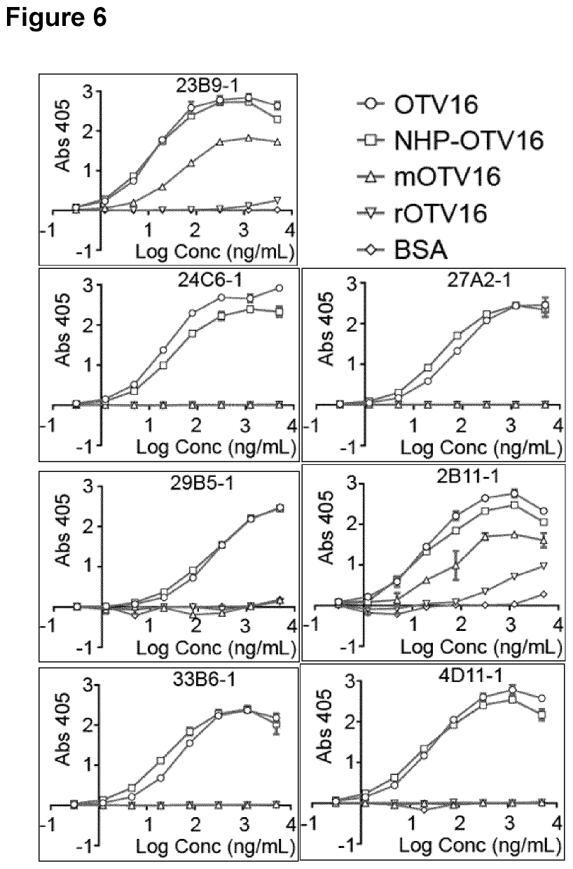

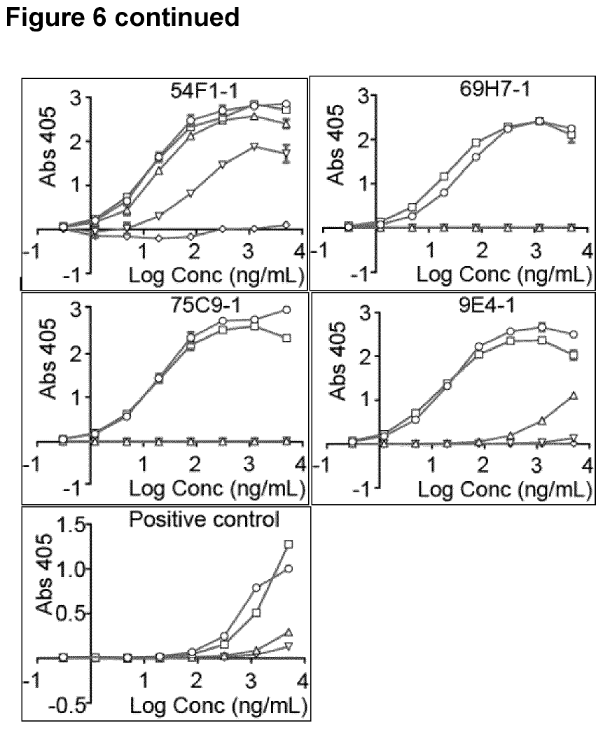

FIG. 6: Peptide ELISA of purified recombinant monoclonal antibodies. Monoclonal antibodies were tested for binding to OTV16 peptide, NHP-OTV16 peptide, mOTV16 peptide and rOTV16 peptide. Non-specific binding was determined using wells coated with bovine serum albumin (BSA). Serum from immunized rabbit (known to contain OTV16-binding antibody) was used as a positive binding control. Serial dilutions of antibody were incubated for 1 hr in ELISA plates coated with peptide or BSA. Plates were washed, and bound antibody was detected using anti-rabbit IgG conjugated to alkaline phosphate with absorbance measured at 405 nm.

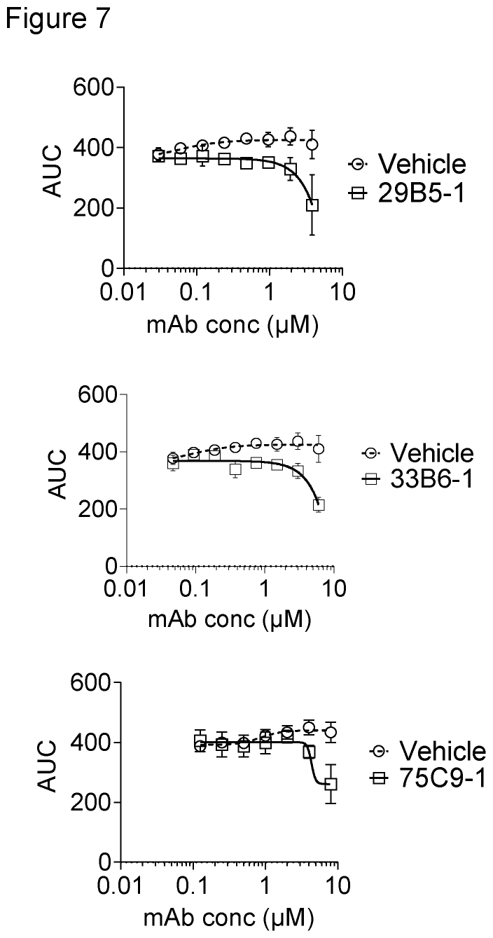

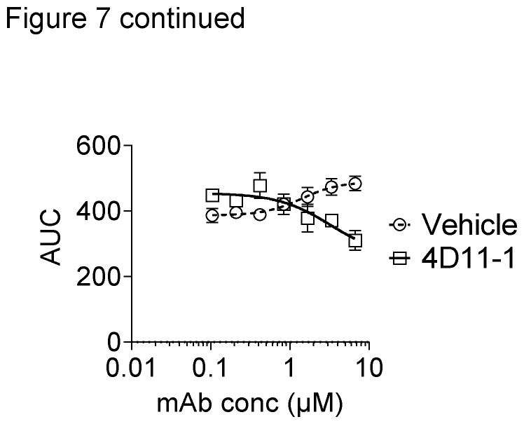

FIG. 7: FLIPR (Fluorescence Imaging Plate Reader) determination of inhibition of TRPV1-mediated capsaicin-induced Ca²⁺ uptake by recombinant monoclonal antibodies. Adherent CHO cells expressing hTRPV1 were loaded with the calcium indicator Calcium-6 for 1 hr at 37° C. followed by washing. Wells were then loaded with serial dilutions of antibody, or vehicle (PBS, pH 7). After 30 mins, capsaicin was added to wells (final capsaicin concentration was EC H). Calcium content within the cells was then monitored for 5 minutes using a plate reader measuring fluorescence. Using the value of the area under the kinetic curve (AUC) for each concentration of antibody (or corresponding vehicle), dose-response curves were built, and a nonlinear regression model (4-parameter) was fitted to the data. N=2 for 29B5-1 (N=4 for the corresponding vehicle), N=2 for 33B6-1 (N=4 for the corresponding vehicle), N=3 for 75C9-1 (N=6 for the corresponding vehicle) and N=2 for 4D11-1 (N=4 for the corresponding vehicle).

### EXAMPLE 1

### Materials and Methods

### Chemicals

Cell culturing medium (DMEM/Ham's F12 with glutamine and Ham's F12), fetal bovine serum, and Accutase were purchased from PAA. Zeocin was purchased from Invitrogen. The control antibody (Rabbit Gamma Globulin—RRID: AB_2532177—catalogue number 31887) was purchased from Thermo Fisher Scientific. Mavatrep and AMG517 were purchased from MedChemExpress. All other chemicals were purchased from Sigma. The following buffers were used; F: 140 mM NaCl, 5 mM KCl, 1 mM CaCl₂), 1 mM MgCl₂ 10 mM HEPES, 10 mM D-glucose, pH 7.4, G: 120 mM KCl, 2 mM MgCl₂, 10 mM HEPES, 10 mM EGTA, pH 7.2. H: 50 mM Tris, 100 mM NaCl, 10 mM MgCl₂, 1 mM EDTA, 0.01% Triton X-100 and 1 mM DTT.

### Cell Culture

Adherent Chinese hamster ovary (CHO) cells with a tetracycline regulated expression system (T-REx) of hTRPV1 (human TRPV1) were cultivated in medium (DMEM/F12 with glutamine) supplemented with 10% fetal bovine serum (FBS), Zeocin (350 μg/ml), and Blasticidin (5 μg/ml). 18-24 hours before use, the cells were incubated in medium supplemented with 10% FBS and Doxycycline (1 μg/ml) in order to induce hTRPV1 expression.

### Antibody Development

For the epitope (peptide) used for antibody generation, a synthetic peptide including additional cysteine residues was synthesized and purified. The synthetic peptide sequence is set forth as SEQ ID NO:3 (OTV16).

The SEQ ID NO:3 (OTV16) peptide used is a cyclic peptide and it was linked to keyhole limpet hemocyanin (KLH) via the propargyl group. The KLH-linked epitope (peptide) was used to produce polyclonal antibodies by immunization of specific pathogen-free (SPF) rabbits following injection of the KLH linked peptides.

The peptides were produced by solid phase peptide synthesis (SPPS) with capping step. Cyclization was done by oxidizing terminal cysteines, creating a disulphide bridge between peptide ends. The cyclic peptides were conjugated by click chemistry using a propargyl group on the peptide and azide on KLH. Antibodies were purified using a Protein G column followed by affinity purification against the peptide.

The antibodies were affinity purified and subjected to an ELISA test. The ELISA test showed that the antibody was able to bind to the respective peptide (i.e. the peptide used to immunize the rabbit to produce the antibody) when the peptide was immobilized on a surface (data not shown).

Generation of both synthetic peptides and polyclonal antibodies were performed by Innovagen AB (Lund, Sweden).

Electrophysiology

### Electrophysiological Patch Clamp Recordings—OTV16

Whole cell recordings were performed using a microfluidic device for patch clamp recordings (Dynaflow, Cellectricon AB, Goteborg, Sweden) together with an Axopatch 200B (Molecular Devices, USA) patch clamp amplifier. The cells were adherent Chinese hamster ovary (CHO) cells, as described above. Bath and pipette (borosilicate glass capillaries, i.d. 0.86 mm; GC150E-7.5, Harvard Apparatus Ltd) solutions contained buffer F and G, respectively. The cells were clamped at −60 mV and the current signals were recorded with a sampling frequency of 10 kHz and low pass filtered at 2 kHz. The patch-clamp recordings were acquired using digital/analogue sampling (Axon Digidata 1550) and acquisition software (Clampex version 10.7, Molecular Devices). In experiments testing OTV16 antibody, AMG 517, Mavatrep and control antibody (ThermoFischer #31887), current amplitudes were measured by exposing cells to capsaicin, with and without antibody or small molecule (the small molecules are AMG 517 and Mavatrep). The cells were exposed to 100 nM capsaicin in buffer F for ˜20 s, followed by buffer F for 60 s, antibody (or small molecule) in buffer F for 60 s and then 100 nM capsaicin together with antibody (or small molecule) in buffer F for ˜20 s.

Measurements where the seal resistance shifted largely during treatment were excluded from analysis.

### Electrophysiology Patch Clamp—Data Analysis—OTV16

For all measurements, the recorded amplitude of the peak during stimulation with antibody+capsaicin was divided by the recorded amplitude of the peak during stimulation with capsaicin. Measurements were performed on cells from at least two different cell culture dishes. Each data point (n) represents a single cell. Normality was assessed using the Shapiro-Wilk test. Data is presented as mean±SEM.

### Calcium Imaging Method for Assessing Inhibition of Capsaicin-Induced Activation of TRPV1

CHO cells expressing hTRPV1 were incubated with 4.4 μM of the calcium indicator Fluo-3 AM for 30 min at 37° C. and thereafter washed and then incubated with various concentrations of antibodies dissolved in PBS (OTV16 n=12 or control antibody n=12 (ThermoFischer #31887)) for 1 h, at room temperature. Calcium content within the cells was then monitored by measuring fluorescence intensity using a plate reader (CLARIOstar, BMG Labtech) before and after application of 1 μM capsaicin and 150 μM Ca²⁺ to the antibody solution covering the cells. The total calcium uptake caused by capsaicin in those samples that were prior incubated with antibodies was normalized against the calcium uptake caused by capsaicin activation (i.e. capsaicin and calcium) only (i.e. with no prior antibody incubation). Data is represented as mean±SEM. Statistical significance was determined using a Kruskal-Wallis test followed by Dunn's multiple comparison.

### Imaging

### Calcium Imaging of Heat Response

In order to measure the antibodies' effect on hTRPV1 heat response (42° C.) an optical heating system was used to deliver heat pulses to cells. The cells were adherent Chinese hamster ovary (CHO) cells, as described above. A commercial microfluidic device, the Biopen Prime (Fluicell AB), was used to deliver the antibodies to cells. Heat increases the open probability of hTRPV1 channels which causes an influx of calcium ions. This influx was measured using the calcium indicator Fluo-3. The experiments were performed in the same glass bottomed Petri dishes as the cells were grown in (50 mm uncoated, MatTek). All cell experiments including Fluo-3 AM incubation were performed using DMEM/F-12, HEPES cell culture medium without phenol red (Gibco). The “AM” ester is initially attached to the Fluo-3 indicator, which makes it cell permeable. Once added to the cell culture medium the Fluo-3 AM enters the cell and the “AM” ester part is cleaved off, leaving the Fluo-3 indicator inside the cell. The Fluo-3 indicator itself is non-cell permeable so it remains inside the cell.

### Confocal Microscopy

Cells were imaged using a Bio-Rad MRC 1024 confocal unit fitted with a dual Calypso laser (Cobolt, Solna, Sweden) attached to a Nikon Diaphot 200 inverted microscope and a Nikon Plan Apo 20× dry objective (N.A. 0.75 Nikon, Tokyo, Japan). Excitation wavelengths used were 491 nm (Fluo-3) and the emitted light was collected through a 522 nM filter. Images were acquired for the full view of the 20× objective. The frame rate was one image per 7 sec and the pixel resolution 1024×1024.

### Heat Probe

A laser heating system was used to locally increase the temperature to 42° C. around selected cells. The laser heating system was built in-house by Fluicell AB. This optical local heating system is based upon a CW 4W 1470-nm semiconductor diode laser (4PN-106, Seminex Corporation, USA) driven by a 20 A benchtop power source (ARO-4320, Arroyo Instruments). This delivers a localized beam to the sample (a group of cells in our case) through a 105 μm core, 0.22 NA, broadband optical fiber (M63L01, Thorlabs). The optical fiber is coupled to a 5 mm fiber optic cannula (CFMLC21L05, 105 μm, 0.22NA, Thorlabs) so that it can be precisely positioned at any desired location in a Petri dish.

A narrow beam of 1470 nm radiation, exiting the tip of the optical fiber, induces local heating of the water within its path. The extent of heating is determined by the beam intensity which is modulated by the current setting of the laser, and the distance between the tip of the fiber and the sample. In this study, the current and distance are optimized to achieve a sample temperature of 42° C. The relationship between distance, applied current and temperature was calibrated using a previously described technique (Wegrzyn, I., et al. An optofluidic temperature probe. Sensors (Switzerland) (2013), 13(4), 4289-4302, doi:10.3390/s130404289), using a fluidic device (Biopen, Fluicell AB), probing the fluorescent responses.

### Biopen

A Biopen Prime (Fluicell AB) was used to deliver antibodies to cells. The Biopen is a free-standing microfluidic device which can be readily positioned using micromanipulators such that the tip can be aligned adjacent to a selected group of cells in a Petri dish, to locally deliver a compound without contamination of the surrounding environment. The solutions to be delivered by the Biopen are loaded into wells on the Biopen to minimize compound consumption. The switching between solutions is controlled by dedicated software.

### Calcium Imaging of Heat Response—Recording

30 min before imaging the cell medium was changed to medium containing 36 μM Fluo-3-AM (F1242, Thermo-Fisher) and the samples were incubated for 30 min at RT and then washed and provided with fresh growth medium which contains Ca²⁺ (DMEM/F-12, HEPES cell culture medium without phenol red (Gibco)). The Biopen was positioned above a group of cells using a micromanipulator. The heat-probe was positioned 10 μm above the dish bottom and at approx. 100 μm distance from the Biopen outlet. To define which cells that are exposed to solution delivery from the Biopen, an initial pulse of Sulforhodamine B was delivered. After the Sulforhodamine B fluorescence declined the cells was optically heated for 7 s (42° C.) and the fluorescence response from Fluo-3 was measured (cell response, Peak #1). Subsequently, antibody solution (OTV16 antibody or control antibody (ThermoFischer #31887)) or small molecule solution (2 μM Mavatrep or 2 μM AMG-517) was delivered for 90 s and a second heat-pulse was applied during the last 7 s of application (cell response, Peak #2).

### Data Analysis of Calcium Imaging of Heat Response

Data analyses were performed in Image J and GraphPad Prism software. The Sulforhodamine B pulse visualizes which cells that are reached by Biopen solution delivery and thereby defining which cell that will be included in the measurement. The fluorescence intensity of these cells was measured and averaged for each time point, to obtain an average curve for the cells stimulated in one experiment. The height of Peak #1 was measured to determine heat response without antibody present, and the height of Peak #2 was measured to determine heat response with antibody present. Peak #2 value was divided with Peak #1 to obtain a ratio. The ratio of the peaks for the OTV16 antibody was compared to the control antibody.

Normality was assessed using Shapiro-Wilk test. Data is presented as mean±SEM. n equals one experiment containing on average 5-10 cells.

### Results and Discussion

A moduli selective antagonistic antibody of TRPV1, named antibody OTV16 was developed as described above (i.e. by immunizing rabbits with the stated KLH-linked OTV16 epitope (peptide)). This moduli selective antibody is capable of preferentially inhibiting capsaicin activation of TRPV1 as opposed to heat activation of TRPV1, thus reducing or avoiding the heat-related side-effects that have been observed with previous small molecule antagonists.

The moduli selective effect of the OTV16 antibody was determined by comparing the degree of inhibition of capsaicin and heat activation of TRPV1, respectively. The activity profiles were compared to those of the small molecule antagonists Mavatrep (Manitpisitkul P, et al. Scand. J. Pain (2018), 18(2):151-164) and AMG-517 (Gavva, N. R. et al. Pain (2008), 136 (1-2), 202-210) (FIG. 1). Inhibition of capsaicin-induced channel activity was evaluated using whole cell patch-clamp recordings and the effect on heat-induced activity was evaluated measuring intracellular Ca²⁺ flux with fluorescence, where the antibody solution was delivered using the Biopen R (Fluicell AB) system.

During the patch-clamp experiments (which evaluated capsaicin-induced TRPV1 activation), cells were exposed to capsaicin followed by antibody (or small molecule) and finally capsaicin in the presence of antibody (or small molecule). During the fluorescence experiments (which evaluated heat-induced TRPV1 activation), cells were exposed to heat (42° C.) followed by antibody (or small molecule) and finally exposed to heat (42° C.) in the presence of antibody (or small molecule).

The level of heat and capsaicin inhibition by the antibodies can be found in FIG. 1, where the antibodies were used at a 5 times higher concentration for the evaluation of inhibition of heat-induced activation as compared to the antibody concentration used for the evaluation of inhibition of capsaicin-induced activation of TRPV1, whereas Mavatrep and AMG517 were evaluated at equal concentrations.

The OTV16 antibody elicited a 35.7% inhibition of capsaicin-induced TRPV1 activation at 26.7 nM and a −0.7% inhibition of temperature (heat)-induced TRPV1 activation at 134 nM (FIG. 1).

Mavatrep elicited a 100% inhibition of capsaicin-induced TRPV1 activation at 100 nM and a 89.3% inhibition of temperature (heat)-induced TRPV1 activation at 100 nM (FIG. 1).

AMG517 elicited a capsaicin inhibition of 100% inhibition of capsaicin-induced TRPV1 activation at 100 nM and a 88.8% inhibition of temperature (heat)-induced TRPV1 activation at 100 nM (FIG. 1).

The control antibody elicited no inhibition of either capsaicin- or temperature-induced activation of TRPV1 (FIG. 1).

All antibody concentrations evaluated can be seen in FIG. 2 (in connection with the inhibition of capsaicin-induced TRPV1 activation) and in FIG. 3 (in connection with the inhibition of temperature (heat)-induced TRPV1 activation).

In summary, the OTV16 antibody demonstrated a moduli-selective activity profile by eliciting a higher inhibition of capsaicin activation compared to heat-activation even though evaluation of heat activation was performed at 5 times higher antibody concentration. In contrast, both Mavatrep and AMG517 inhibited capsaicin and heat activation at close to equal levels at 100 nM. This clearly demonstrates that the OTV16 antibody is more than a simple TRPV1 antagonist; it selectivity inhibits capsaicin induced activation of TRPV1 as opposed to heat-induced activation of TRPV1. The OTV16 antibody is a promising new candidate for TRPV1-targeted pain therapy and is currently pursued as a drug candidate. These would not only be the first therapeutic anti-TRPV1 antibodies, they would also be the first therapeutic antibodies ever developed for an ion channel. The present data also establishes that epitopes on TRPV1 that correspond to (or correspond essentially to) the epitope (peptide) amino acid sequence used to generate the OTV16 antibody are useful epitopes to target (i.e. to generate antibodies against) in order to identify moduli selective antibodies as described herein.

FIG. 4 also shows that the OTV16 polyclonal antibody is able to significantly inhibit capsaicin induced activation of TRPV1. For the FIG. 4 experiments, rather than assessing capsaicin induced TRPV1 activation by the patch-clamp method, calcium imaging was utilized and FIG. 4 shows that the OTV16 antibody is able to inhibit capsaicin-induced calcium uptake. Cells expressing hTRPV1 were incubated with 4.4 μM Fluo-3 AM for 30 min. Cells were thereafter washed and incubated for 1 h with antibodies dissolved in PBS, at varying concentrations. Fluorescence intensity was measured before and after application of 1 μM capsaicin and 150 μM Ca²⁺ using a CLARIOstar (BMG Labtech) micro plate reader.

It is evident from the above that the inventors have identified certain peptide sequences (epitope sequences) that correspond to (or correspond essentially to) epitopes on TRPV1 that are very useful to target with antibodies in order to achieve inhibition of TRPV1. In particular, the inventors have identified epitopes on TRPV1 that are very useful to target with antibodies in order to achieve preferential inhibition of capsaicin-induced activation of TRPV1 as opposed to heat-induced activation of TRPV1. Antibodies with such selectivity for inhibition of the capsaicin axis of TRPV1 will be therapeutically advantageous as avoiding (or reducing) concomitant inhibition of the heat axis of TRPV1 would avoid (or reduce) adverse effects such as hyperthermia or loss of heat sensation that are observed with other TRPV1 inhibitors (as discussed elsewhere herein).

### EXAMPLE 2

This Example describes the generation of and testing of the 23B9-1, 24C6-1, 27A2-1, 29B5-1, 2B₁₁-1, 33B6-1, 4D11-1, 54F1-1, 69H7-1, 75C9-1 and 9E4-1 antibodies.

### Materials and Methods

### Production of Monoclonal Antibodies (Rabbit IgG) Using B-Cell Cloning Technology

Peptide synthesis was performed as described in Example 1 herein. Cyclization of the OTV16 peptide (SEQ ID NO:3) was done by oxidizing terminal cysteines, creating a disulphide bridge between peptide ends. The cyclic peptide was conjugated (linked) to the keyhole limpet hemocyanin (KLH) by click chemistry using a propargyl group on the peptide and azide on KLH. New Zealand rabbits were immunized with the OTV16 peptide of SEQ ID NO:3 linked to keyhole limpet hemocyanin (KLH). Immune responses were evaluated using ELISA. After the immunization process, rabbits were selected based on ELISA screening of serum, and peripheral blood mononuclear cells (PBMC) were used for isolating antigen positive B-cells. Antigen positive B-cells were plated in 96-well plates and allowed to grow and produce antibodies. Supernatants were screened against antigen peptide by indirect ELISA.

### Cloning and Sequencing of Rabbit IgG, and Generation of Recombinant Antibodies

From antigen positive B-cells, RNA was prepared from which cDNA was synthesized. Variable Light (VL) and Variable Heavy (VH) regions of cDNA were amplified and cloned into standard cloning vector separately. Identification of positive clones was done by colony PCR followed by gel electrophoresis. VL and VH DNA and amino acid sequences were obtained from positive clones. A total of 26 B-cell clones were sequenced. Eleven unique monoclonal antibody sequences were identified, viz. 23B9-1, 24C6-1, 27A2-1, 29B5-1, 2B₁₁-1, 33B6-1, 4D11-1, 54F1-1, 69H7-1, 75C9-1, 9E4-1. Sequences of the 23B9-1, 24C6-1, 27A2-1, 29B5-1, 2B₁₁-1, 33B6-1, 4D11-1, 54F1-1, 69H7-1, 75C9-1 and 9E4-1 antibodies are set forth herein in Tables A-K.

To generate recombinant monoclonal 23B9-1, 24C6-1, 27A2-1, 29B5-1, 2B₁₁-1, 33B6-1, 4D11-1, 54F1-1, 69H7-1, 75C9-1 and 9E4-1 antibodies, VH and VL sequences of the antibodies were cloned into expression vectors and co-transfected into a mammalian expression system (human embryonic kidney (HEK) cells). Cell culture supernatant containing transient expressed antibodies was collected. Recombinant antibody was then purified on a Protein A resin into 0.01M PBS, pH 7.0. The recombinant antibodies are IgG antibodies. In these recombinant antibodies, the VH and VL domain amino acid sequences are rabbit sequences, and the constant regions are rabbit IgG constant region sequences (the recombinant backbone was chosen to be rabbit IgG). The type of light chain of the recombinant IgG antibodies is rabbit kappa. Thus, the recombinant antibodies generated and used in this example are recombinant rabbit IgG antibodies.

### B-Cell ELISA Screening

96-well plates suitable for ELISA were coated with respective peptide (1 μg/mL) in PBS and incubated 1 h at 37° C. or overnight at 4° C. The peptides used to coat the plates were OTV16 (SEQ ID NO:3), NHP-OTV16 (SEQ ID NO:5), and rOTV16 (SEQ ID NO:7) (described below). Plates were washed with wash buffer and incubated 1 h at 37° C. in the presence of blocking buffer (PBS, 0.5% BSA, 0.05% Tween 20, pH 7.4). B-cell supernatants were added and incubated for 1 h at 37° C. Plates were washed with wash buffer and Mouse Anti-Rabbit IgG Fc Antibody conjugated to horseradish peroxidase (Genscript, cat #A01856) was added and incubated for 30 minutes, 37° C. Plates were washed with wash buffer followed by addition of substrate solution, 100 μl/well and incubated for 13-20 minutes at RT. Reaction was stopped by adding 100 μl/well of stop solution. Absorbance was measured at 450 nm. Serum from immunized rabbit known to contain antibody that binds to OTV16 peptide was used as a positive control. B-cell culture medium (antibody-free) was used as a negative control.

As indicated above, the peptides used as bait in the ELISA B-cell supernatant screenings were OTV16 (SEQ ID NO:3), NHP-OTV16 (SEQ ID NO:5), and rOTV16 (SEQ ID NO:7). Supernatants were screened against each of the peptides. The amino sequence of the OTV16 peptide (SEQ ID NO:3) comprises the amino acid sequence YRPVDGLPPFKMEKTG (SEQ ID NO:2), which corresponds to residues 455-470 of human TRPV1 (SEQ ID NO:1). (SEQ ID NO:2 has the amino acid sequence of the OTV16 peptide of SEQ ID NO:3 but without the N-terminal cysteine residue and without the C-terminal cysteine residue). The sequence of NHP-OTV16 used here was (Pra-)CYRPVDGLPPFKMEKTEC(-CONH2) and is synthesized as a cyclic peptide (NHP=non-human primate). The NHP-OTV16 peptide is used here to determine binding to the equivalent region in Macaca mulatta TRPV1. Macaca mulatta TRPV1 is a non-human primate (NHP) TRPV1 sequence. The sequence of rOTV16 used here was (Pra-)CYRPVEGLPPYKLKNTGC(-CONH2), synthesized as a cyclic peptide. The rOTV16 peptide corresponds essentially to the analogous region in rat TRPV1. Screening against a multitude of species variants of the same peptide provides information on antibody cross-reactivity.

### Monoclonal Antibody ELISA Screening

96-well plates suitable for ELISA were coated with peptide (1 μg/mL) in PBS and incubated 1 h at 37° C. The peptides used to coat the plates were OTV16 (SEQ ID NO:3; described above), NHP-OTV16 (SEQ ID NO:5; described above), rOTV16 (SEQ ID NO:7; described above) and mOTV16 (SEQ ID NO:9; described below). Plates were washed with PBS containing 0.05% Tween and incubated 1 h at 37° C. in the presence of blocking buffer (PBS, 0.5% BSA, 0.05% Tween 20, pH 7.4). A serial dilution of recombinant antibodies of the invention (23B9-1 or 24C6-1 or 27A2-1 or 29B5-1 or 2B₁₁-1 or 33B6-1 or 4D11-1 or 54F1-1 or 69H7-1 or 75C9-1 or 9E4-1) was added in blocking buffer and incubated for 1 h at RT. Plates were washed with PBS containing 0.05% Tween and a secondary antibody (anti-rabbit IgG conjugated to Alkaline Phosphatase (DAKO)), was added and incubated for 1 h at RT. Plates were washed with PBS containing 0.05% Tween followed by addition of 1 mg/mL pNPP (p-nitrophenyl 15 phosphate) in pNPP buffer. Absorbance was measured at 405 nm. Serum from rabbit immunized with OTV16 peptide was used as a positive control. Non-specific binding, e.g. to plastic, was determined using wells coated with bovine serum albumin (BSA) protein.

As indicated above, the peptides used as bait in the recombinant monoclonal antibody ELISA screenings were OTV16 (SEQ ID NO:3), NHP-OTV16 (SEQ ID NO:5), rOTV16 (SEQ ID NO:7) and mOTV16 (SEQ ID NO:9). The sequence of mOTV16 used here (SEQ ID NO:9) was (Pra-)CYRPVEGLPPYKLNNTVGC(-CONH2), synthesized as a cyclic peptide. The mOTV16 peptide is used here to determine binding to the analogous region in mouse TRPV1. Recombinant antibody preparations were screened against each of the peptides. Screening against a multitude of species variants of the same epitope provides information on antibody cross-reactivity.

### Measurement of Capsaicin-Induced Activity Using Fluorescence Imaging Plate Reader (FLIPR)

### Cell Culture

Adherent CHO cells with a tetracycline-regulated expression (T-REx) system of human TRPV1 were maintained in Dulbecco's modified Eagle's medium (DMEM)/F12 GlutaMAX (GIBCO, 10565018) supplemented with 10% fetal bovine serum (GIBCO, 10270106), 350 μg/ml Zeocin (Gibco, R25005), 13.6 μg/ml Blasticidin (Gibco, A1113902) and under 95% 02, 5% 002 at 37° C.

### Calcium Influx Assay

For calcium mobilization assays CHO human TRPV1 cells were seeded into poly-D-Lysine coated (Gibco, A3890401) 96-well black-wall, clear-bottom plates (Costar 3606). Microplates were maintained under 95% 02, 5% 002 at 37° C. until fully confluent (24-72 h). TRPV1 receptor expression was induced using 1 μg/ml Doxycycline (Cayman Chemicals, 14422) and experiments were performed 19-24 hrs after induction.

Calcium influx was measured in a plate reader (CLARIOstar Plus, BMG LABTECH) using Calcium-6 assay kit (Molecular Devices, R8191). The assay was performed using a HEPES-buffered salt solution pH 7.4, containing in mM: 140 NaCl, 5 KCl, 10 Glucose, 10 HEPES, 1 MgCl₂, 1 CaCl₂). Calcium-6 loading 1× solution was prepared according to the manufacturers' instructions using the HEPES-buffered salt solution, pH 7.4. Calcium-6 is a calcium indicator (Calcium-6 has an AM (acetoxymethyl ester) portion that provides the hydrophobicity necessary to cross the plasma membrane and enter the cell, and then the AM portion is cleaved intracellularly thus trapping the indicator in the cytosol). Briefly, cell culture media in the 96-well plates was replaced with 100 μl/well of 1× Calcium-6 loading solution (which contains Calcium-6 indicator) and incubated in the dark for one hour at 37° C., 5% CO₂. Meanwhile, serial dilutions (1:2) of either recombinant antibodies or vehicle (PBS, pH 7) were prepared starting from the antibody stock concentration using HEPES-buffered salt solution pH 7.4 (i.e. HEPES-buffered salt solution pH 7.4 was used as the diluent to dilute the recombinant antibodies and the vehicle). Prior to dilutions the antibodies were in PBS solutions, i.e. the stock antibody solutions were antibody-PBS solutions). After Calcium-6 loading, the plate was removed from the incubator and equilibrated to RT in the dark for 10-15 min. Calcium-6 loading solution was then removed and 100 μl/well of each serial dilution (1:2) of either recombinant antibodies or vehicle (PBS pH 7) was loaded into the columns of the 96-well plate and an initial measurement of fluorescence intensity (F₀) was performed (λₑₓ=485 nm, λₑₘ=525 nm; 4 cycles; 12 seconds per cycle; 10 flashes per cycle). Antibodies were then preincubated for 30 minutes at 25° C. inside the plate reader. During antibody preincubation, Capsaicin EC₅₀ was determined; for this purpose, in separate wells (not loaded with antibodies or vehicle) a full dose-response curve for capsaicin (Millipore, 211275) was performed and the EC₅₀ was calculated using CLARIOstar Mars Software (BMG LABTECH). The curve (8 points) was prepared using 1:3 serial dilutions of capsaicin with a starting concentration of 300 nM using HEPES-buffered salt solution pH 7.4. After adding capsaicin serial dilutions, the change in fluorescence intensity was followed for 5 minutes using kinetic reading mode (λₑₓ=485 nm, λₑₘ=525 nm; 100 cycles; 3 seconds per cycle; 10 flashes per cycle). Following antibody preincubation, the effect of recombinant antibodies was assessed by adding 100 μl/well of a 2× capsaicin EC₅₀ solution prepared using HEPES-buffered salt solution pH 7.4 and the change in fluorescence intensity was followed for 5 minutes using kinetic reading mode (λₑₓ=485 nm, λₑₘ=525 nm; 25 cycles; 12 seconds per cycle; 10 flashes per cycle). Data were analysed using GraphPad Prism 9 software (GraphPad Software Inc., San Diego, CA). Briefly, kinetic curves of calcium influx after capsaicin EC₅₀ addition were plotted over time in terms of the ratio ΔF(t)/F₀ (where ΔF(t)=Fₜ−F₀; Fₜ is the fluorescence intensity after capsaicin EC₅₀ addition at the time t, and F₀ is the initial fluorescence intensity measured immediately after antibody loading). Here t=0 to 300 seconds (i.e. to generate the kinetic curve a ratio was calculated for a time point “t” every 12 seconds over the 5 minute time course, i.e. “for t=0 seconds, for t=12 seconds, for t=24 seconds, etc. until t=300 seconds). From the kinetic curves, the value of the area under the curve (AUC) for each concentration of antibody was calculated and dose-response curves were built. Finally, a nonlinear regression model (4-parameter) was fitted to the data.

### Results and Discussion

This example describes the production and testing of recombinant monoclonal antibodies. Rabbits were immunized with OTV16 peptide (SEQ ID NO:3) and individuals having an antigen-specific immune response were selected based on ELISA screening of serum. From these individuals, antigen positive B-cells were isolated from peripheral blood mononuclear cells (PBMC). Antigen-specific B-cells were selected based on ELISA screening of cell culture supernatants.

Eleven B-cell clones were selected based on peptide ELISA of B-cell clone supernatant. All selected clones, 23B9-1, 24C6-1, 27A2-1, 29B5-1, 2B₁₁-1, 33B6-1, 4D11-1, 54F1-1, 69H7-1, 75C9-1, 9E4-1, produced antibody that bound to both OTV16 peptide and NHP-OTV16, and two of these clones, 2B₁₁-1 and 54F1-1, produced antibody that also bound to rOTV16, as shown in FIG. 5.

The VH and VL chains of the eleven B-cell clones were sequenced, and these sequences were introduced into a mammalian cell expression system. Recombinant antibody (rabbit IgG) was then produced, purified and tested by ELISA for binding to OTV16 peptide, and also to non-human primate (NHP-OTV16), mouse (mOTV16) and rat (rOTV16) equivalents of the OTV16 peptide.

After cloning of antibody sequences from the eleven individual B-cell clones into a mammalian expression system, monoclonal recombinant antibody was expressed and purified on Protein A resin. Monoclonal recombinant antibody preparations were screened by peptide ELISA for their ability to bind to OTV16 peptide, NHP-OTV16, mOTV16 and rOTV16. It was confirmed that all recombinant antibody preparations, 23B9-1, 24C6-1, 27A2-1, 29B5-1, 2B₁₁-1, 33B6-1, 4D11-1, 54F1-1, 69H7-1, 75C9-1, 9E4-1, bound to both OTV16 and NHP-OTV16, as shown in FIG. 6, with monoclonal recombinant antibody preparations of 2B₁₁-1 and 54F1-1 also binding to rOTV16. The binding pattern mirrored that seen at the B-cell clone supernatant screening stage, demonstrating therefore that the cloning and recombination procedure had been successful. In addition, monoclonal recombinant antibody preparations of 23B9-1, 2B₁₁-1, 54F1-1 and 9E4-1 showed a degree of binding also to mOTV16. Species cross-reactivity can be useful for efficacy testing during the pre-clinical phase of an antibody development programme, where normally non-human animal models are used.

Recombinant monoclonal antibodies 29B5-1, 33B6-1, 75C9-1 and 4D11-1 were tested for their ability to inhibit capsaicin-induced activation of the full length human TRPV1 protein (hTRPV1) expressed at the plasma membrane in living cells. FLIPR was used to determine the capacity of the monoclonal antibodies 29B5-1, 33B6-1, 75C9-1 and 4D11-1 to inhibit TRPV1-mediated capsaicin-induced Ca²⁺ uptake in hTRPV1-expressing CHO cells. 29B5-1, 33B6-1, 75C9-1 and 4D11-1 were shown to inhibit capsaicin-induced activation of human TRPV1 as compared to controls with no antibody (vehicle), as shown in FIG. 7.

1. An antibody which binds to TRPV1, wherein said antibody binds to an epitope of TRPV1 that is in the region of TRPV1 defined by amino acid residues 455-470 of TRPV1 (SEQ ID NO:1).

2. The antibody of claim 1, wherein said antibody binds to an isolated peptide, said isolated peptide comprising an amino acid sequence of SEQ ID NO:2, or comprising a sequence that has 1, 2, or 3 amino acid substitutions or additions or deletions compared with said amino acid sequence.

3. The antibody of claim 1 or claim 2, wherein said antibody binds to an isolated peptide, said isolated peptide comprising an amino acid sequence of SEQ ID NO:3, or comprising a sequence that has 1, 2, or 3 amino acid substitutions or additions or deletions compared with said amino acid sequence.

4. The antibody of any one of claims 1 to 3, wherein said antibody binds to an isolated peptide, said isolated peptide consisting of an amino acid sequence of SEQ ID NO:2, or consisting of a sequence that has 1, 2, or 3 amino acid substitutions or additions or deletions compared with said amino acid sequence.

5. The antibody of any one of claims 1 to 4, wherein said antibody binds to an isolated peptide, said isolated peptide consisting of an amino acid sequence of SEQ ID NO:3, or consisting of a sequence that has 1, 2, or 3 amino acid substitutions or additions or deletions compared with said amino acid sequence.

6. The antibody of any one of claims 1 to 5, wherein said antibody binds to an isolated peptide, said isolated peptide consisting of an amino acid sequence of SEQ ID NO:3.

7. The antibody of any one of claims 2 to 6, wherein said isolated peptides are cyclic peptides.

8. The antibody of any one of claims 1 to 7, wherein said antibody binds to a conjugate, said conjugate comprising an isolated peptide as defined in any one of claims 2 to 7 and a peptide carrier, preferably said peptide carrier is keyhole limpet hemocyanin (KLH).

9. The antibody of any one of claims 1 to 8, wherein said antibody comprises at least one heavy chain variable region that comprises three CDRs and at least one light chain variable region that comprises three CDRs, wherein said light chain variable region comprises a variable light (VL) CDR1 that has the amino acid sequence of SEQ ID NO:211 or comprises a variable light (VL) CDR1 that has the amino acid sequence of SEQ ID NO:213.

10. The antibody of any one of claims 1 to 9, wherein said antibody comprises at least one heavy chain variable region that comprises three CDRs and at least one light chain variable region that comprises three CDRs, wherein said light chain variable region comprises a variable light (VL) CDR3 that has the amino acid sequence of SEQ ID NO:215, or comprises a variable light (VL) CDR3 that has the amino acid sequence of SEQ ID NO:217.

11. The antibody of any one of claims 1 to 10, wherein said antibody comprises at least one heavy chain variable region that comprises three CDRs and at least one light chain variable region that comprises three CDRs, wherein said light chain variable region comprises a VL CDR1 that has the amino acid sequence of SEQ ID NO:211 and comprises a VL CDR3 that has the amino acid sequence of SEQ ID NO:215.

12. The antibody of any one of claims 1 to 10, wherein said antibody comprises at least one heavy chain variable region that comprises three CDRs and at least one light chain variable region that comprises three CDRs, wherein said light chain variable region comprises a VL CDR1 that has the amino acid sequence of SEQ ID NO:213 and comprises a VL CDR3 that has the amino acid sequence of SEQ ID NO:217.

13. The antibody of any one of claims 1 to 12, wherein said antibody comprises at least one heavy chain variable region that comprises three CDRs and at least one light chain variable region that comprises three CDRs, wherein
(i) said heavy chain variable region comprises a variable heavy (VH) CDR1 that has the amino acid sequence of SEQ ID NO:125, a VH CDR2 that has the amino acid sequence of SEQ ID NO:126 and a VH CDR3 that has the amino acid sequence of SEQ ID NO:127, or sequences substantially homologous thereto, and/or wherein said light chain variable region comprises a variable light (VL) CDR1 that has the amino acid sequence of SEQ ID NO:128, a VL CDR2 that has the amino acid sequence of SEQ ID NO:129 and a VL CDR3 that has the amino acid sequence of SEQ ID NO:130, or sequences substantially homologous thereto;
(ii) said heavy chain variable region comprises a variable heavy (VH) CDR1 that has the amino acid sequence of SEQ ID NO:35, a VH CDR2 that has the amino acid sequence of SEQ ID NO:36 and a VH CDR3 that has the amino acid sequence of SEQ ID NO:37, or sequences substantially homologous thereto, and/or wherein said light chain variable region comprises a variable light (VL) CDR1 that has the amino acid sequence of SEQ ID NO:38, a VL CDR2 that has the amino acid sequence of SEQ ID NO:39 and a VL CDR3 that has the amino acid sequence of SEQ ID NO:40, or sequences substantially homologous thereto;
(iii) said heavy chain variable region comprises a variable heavy (VH) CDR1 that has the amino acid sequence of SEQ ID NO:53, a VH CDR2 that has the amino acid sequence of SEQ ID NO:54 and a VH CDR3 that has the amino acid sequence of SEQ ID NO:55, or sequences substantially homologous thereto, and/or wherein said light chain variable region comprises a variable light (VL) CDR1 that has the amino acid sequence of SEQ ID NO:56, a VL CDR2 that has the amino acid sequence of SEQ ID NO:57 and a VL CDR3 that has the amino acid sequence of SEQ ID NO:58, or sequences substantially homologous thereto;
(iv) said heavy chain variable region comprises a variable heavy (VH) CDR1 that has the amino acid sequence of SEQ ID NO:71, a VH CDR2 that has the amino acid sequence of SEQ ID NO:72 and a VH CDR3 that has the amino acid sequence of SEQ ID NO:73, or sequences substantially homologous thereto, and/or wherein said light chain variable region comprises a variable light (VL) CDR1 that has the amino acid sequence of SEQ ID NO:74, a VL CDR2 that has the amino acid sequence of SEQ ID NO:75 and a VL CDR3 that has the amino acid sequence of SEQ ID NO:76, or sequences substantially homologous thereto;
(v) said heavy chain variable region comprises a variable heavy (VH) CDR1 that has the amino acid sequence of SEQ ID NO:89, a VH CDR2 that has the amino acid sequence of SEQ ID NO:90 and a VH CDR3 that has the amino acid sequence of SEQ ID NO:91, or sequences substantially homologous thereto, and/or wherein said light chain variable region comprises a variable light (VL) CDR1 that has the amino acid sequence of SEQ ID NO:92, a VL CDR2 that has the amino acid sequence of SEQ ID NO:93 and a VL CDR3 that has the amino acid sequence of SEQ ID NO:94, or sequences substantially homologous thereto;
(vi) said heavy chain variable region comprises a variable heavy (VH) CDR1 that has the amino acid sequence of SEQ ID NO:107, a VH CDR2 that has the amino acid sequence of SEQ ID NO:108 and a VH CDR3 that has the amino acid sequence of SEQ ID NO:109, or sequences substantially homologous thereto, and/or wherein said light chain variable region comprises a variable light (VL) CDR1 that has the amino acid sequence of SEQ ID NO:110, a VL CDR2 that has the amino acid sequence of SEQ ID NO:111 and a VL CDR3 that has the amino acid sequence of SEQ ID NO:112, or sequences substantially homologous thereto;
(vii) said heavy chain variable region comprises a variable heavy (VH) CDR1 that has the amino acid sequence of SEQ ID NO:17, a VH CDR2 that has the amino acid sequence of SEQ ID NO:18 and a VH CDR3 that has the amino acid sequence of SEQ ID NO:19, or sequences substantially homologous thereto, and/or wherein said light chain variable region comprises a variable light (VL) CDR1 that has the amino acid sequence of SEQ ID NO:20, a VL CDR2 that has the amino acid sequence of SEQ ID NO:21 and a VL CDR3 that has the amino acid sequence of SEQ ID NO:22, or sequences substantially homologous thereto;
(viii) said heavy chain variable region comprises a variable heavy (VH) CDR1 that has the amino acid sequence of SEQ ID NO:143, a VH CDR2 that has the amino acid sequence of SEQ ID NO:144 and a VH CDR3 that has the amino acid sequence of SEQ ID NO:145, or sequences substantially homologous thereto, and/or wherein said light chain variable region comprises a variable light (VL) CDR1 that has the amino acid sequence of SEQ ID NO:146, a VL CDR2 that has the amino acid sequence of SEQ ID NO:147 and a VL CDR3 that has the amino acid sequence of SEQ ID NO:148, or sequences substantially homologous thereto;
(ix) said heavy chain variable region comprises a variable heavy (VH) CDR1 that has the amino acid sequence of SEQ ID NO:161, a VH CDR2 that has the amino acid sequence of SEQ ID NO:162 and a VH CDR3 that has the amino acid sequence of SEQ ID NO:163, or sequences substantially homologous thereto, and/or wherein said light chain variable region comprises a variable light (VL) CDR1 that has the amino acid sequence of SEQ ID NO:164, a VL CDR2 that has the amino acid sequence of SEQ ID NO:165 and a VL CDR3 that has the amino acid sequence of SEQ ID NO:166, or sequences substantially homologous thereto;
(x) said heavy chain variable region comprises a variable heavy (VH) CDR1 that has the amino acid sequence of SEQ ID NO:179, a VH CDR2 that has the amino acid sequence of SEQ ID NO:180 and a VH CDR3 that has the amino acid sequence of SEQ ID NO:181, or sequences substantially homologous thereto, and/or wherein said light chain variable region comprises a variable light (VL) CDR1 that has the amino acid sequence of SEQ ID NO:182, a VL CDR2 that has the amino acid sequence of SEQ ID NO:183 and a VL CDR3 that has the amino acid sequence of SEQ ID NO:184, or sequences substantially homologous thereto;
(xi) said heavy chain variable region comprises a variable heavy (VH) CDR1 that has the amino acid sequence of SEQ ID NO:197, a VH CDR2 that has the amino acid sequence of SEQ ID NO:198 and a VH CDR3 that has the amino acid sequence of SEQ ID NO:199, or sequences substantially homologous thereto, and/or wherein said light chain variable region comprises a variable light (VL) CDR1 that has the amino acid sequence of SEQ ID NO:200, a VL CDR2 that has the amino acid sequence of SEQ ID NO:201 and a VL CDR3 that has the amino acid sequence of SEQ ID NO:202, or sequences substantially homologous thereto; or
wherein said substantially homologous sequences are sequences containing 1, 2 or 3 amino acid substitutions compared to the given CDR sequences, or said substantially homologous sequence is a sequence containing conservative amino acid substitutions of the given CDR sequences.

14. The antibody of claim 9, wherein said antibody comprises a VL CDR1 has the amino acid sequence of SEQ ID NO:211 and comprises a VL CDR2, a VL CDR3, a VH CDR1, a VH CDR2 and a VH CDR3 that have amino acid sequences as defined together in any one of parts (i) to (iv), (vi) or (ix) to (xi) of claim 13.

15. The antibody of claim 10, wherein said antibody comprises a VL CDR3 that has the amino acid sequence of SEQ ID NO:215 and comprises a VL CDR1, a VL CDR2, a VH CDR1, a VH CDR2 and a VH CDR3 that have amino acid sequences as defined together in any one of parts (i), (iii), (iv), (vi) or (ix) to (xi) of claim 13.

16. The antibody of claim 9 or claim 10, wherein said antibody comprises a VL CDR1 that has the amino acid sequence of SEQ ID NO:211 and comprises a VL CDR3 that has the amino acid sequence of SEQ ID NO:215, and further comprises a VL CDR2, a VH CDR1, a VH CDR2 and a VH CDR3 that have amino acid sequences as defined together in any one of parts (i), (iii), (iv), (vi) or (ix) to (xi) of claim 13.

17. The antibody of claim 9, wherein said antibody comprises a VL CDR1 has the amino acid sequence of SEQ ID NO:213 and comprises a VL CDR2, a VL CDR3, a VH CDR1, a VH CDR2 and a VH CDR3 that have amino acid sequences as defined together in any one of parts (v), (vii) or (viii) of claim 13.

18. The antibody of claim 10, wherein said antibody comprises a VL CDR3 has the amino acid sequence of SEQ ID NO:217 and comprises a VL CDR1, a VL CDR2, a VH CDR1, a VH CDR2 and a VH CDR3 that have amino acid sequences as defined together in any one of parts (v), (vii) or (viii) of claim 13.

19. The antibody of claim 9 or claim 10, wherein said antibody comprises a VL CDR1 that has the amino acid sequence of SEQ ID NO:213 and comprises a VL CDR3 that has the amino acid sequence of SEQ ID NO:217, and further comprises a VL CDR2, a VH CDR1, a VH CDR2 and a VH CDR3 that have amino acid sequences as defined together in any one of parts (v), (vii) or (viii) of claim 13.

20. The antibody of any one of claims 1 to 19, wherein said antibody comprises at least one heavy chain variable region that comprises three CDRs and at least one light chain variable region that comprises three CDRs, wherein
(i) the light chain variable region has the amino acid sequence of SEQ ID NO:124, or a sequence having at least 80% sequence identity thereto and/or wherein the heavy chain variable region has the amino acid sequence of SEQ ID NO:123, or a sequence having at least 80% sequence identity thereto;
(ii) the light chain variable region has the amino acid sequence of SEQ ID NO:34, or a sequence having at least 80% sequence identity thereto and/or wherein the heavy chain variable region has the amino acid sequence of SEQ ID NO:33, or a sequence having at least 80% sequence identity thereto;
(iii) the light chain variable region has the amino acid sequence of SEQ ID NO:52, or a sequence having at least 80% sequence identity thereto and/or wherein the heavy chain variable region has the amino acid sequence of SEQ ID NO:51, or a sequence having at least 80% sequence identity thereto;
(iv) the light chain variable region has the amino acid sequence of SEQ ID NO:70, or a sequence having at least 80% sequence identity thereto and/or wherein the heavy chain variable region has the amino acid sequence of SEQ ID NO:69, or a sequence having at least 80% sequence identity thereto;
(v) the light chain variable region has the amino acid sequence of SEQ ID NO:88, or a sequence having at least 80% sequence identity thereto and/or wherein the heavy chain variable region has the amino acid sequence of SEQ ID NO:87, or a sequence having at least 80% sequence identity thereto;
(vi) the light chain variable region has the amino acid sequence of SEQ ID NO:106, or a sequence having at least 80% sequence identity thereto and/or wherein the heavy chain variable region has the amino acid sequence of SEQ ID NO:105, or a sequence having at least 80% sequence identity thereto;
(vii) the light chain variable region has the amino acid sequence of SEQ ID NO:16, or a sequence having at least 80% sequence identity thereto and/or wherein the heavy chain variable region has the amino acid sequence of SEQ ID NO:15, or a sequence having at least 80% sequence identity thereto;
(viii) the light chain variable region has the amino acid sequence of SEQ ID NO:142, or a sequence having at least 80% sequence identity thereto and/or wherein the heavy chain variable region has the amino acid sequence of SEQ ID NO:141, or a sequence having at least 80% sequence identity thereto;
(ix) the light chain variable region has the amino acid sequence of SEQ ID NO:160, or a sequence having at least 80% sequence identity thereto and/or wherein the heavy chain variable region has the amino acid sequence of SEQ ID NO:159, or a sequence having at least 80% sequence identity thereto;
(x) the light chain variable region has the amino acid sequence of SEQ ID NO:178, or a sequence having at least 80% sequence identity thereto and/or wherein the heavy chain variable region has the amino acid sequence of SEQ ID NO:177, or a sequence having at least 80% sequence identity thereto; or
(xi) the light chain variable region has the amino acid sequence of SEQ ID NO:196, or a sequence having at least 80% sequence identity thereto and/or wherein the heavy chain variable region has the amino acid sequence of SEQ ID NO:195, or a sequence having at least 80% sequence identity thereto.

21. The antibody of any one of claims 1 to 20, wherein said antibody is a monoclonal antibody or a polyclonal antibody.

22. The antibody of any one of claims 1 to 21, wherein said antibody is a whole antibody comprising an antibody constant region.

23. The antibody of any one of claims 1 to 22, wherein said antibody is an IgG antibody.

24. The antibody of any one of claims 1 to 21, wherein said antibody is an antigen binding fragment of an antibody.

25. The antibody of any one of claims 1 to 24, wherein said antibody inhibits capsaicin-induced activation of TRPV1.

26. The antibody of any one of claims 1 to 25 wherein said antibody preferentially inhibits capsaicin-induced activation of TRPV1 as opposed to heat-induced activation of TRPV1.

27. The antibody of any one of claims 1 to 26, wherein said antibody binds to human TRPV1 and binds to Macaca mulatta TRPV1.

28. The antibody of any one of claims 1 to 27, wherein said antibody binds to human TRPV1, binds to Macaca mulatta TRPV1 and binds to mouse TRPV1.

29. The antibody of any one of claims 1 to 27, wherein said antibody binds to human TRPV1, binds to Macaca mulatta TRPV1 and binds to rat TRPV1.

30. The antibody of any one of claims 1 to 29, wherein said antibody binds to human TRPV1, binds to Macaca mulatta TRPV1, binds to mouse TRPV1 and binds to rat TRPV1.

31. A composition comprising an antibody of any one of claims 1 to 30 and a diluent, carrier or excipient, preferably a pharmaceutically acceptable diluent, carrier or excipient.

32. A nucleic acid molecule comprising a nucleotide sequence that encodes an antibody of any one of claims 1 to 30, or a set of nucleic acid molecules each comprising a nucleotide sequence, wherein said set of nucleic acid molecules together encode an antibody of any one of claims 1 to 30.

33. A method of producing an antibody according to any one of claims 1 to 30, comprising the steps of:
(i) culturing a host cell comprising (i) one or more nucleic acid molecules encoding an antibody according to any one of claims 1 to 30 or (ii) a set of nucleic acid molecules each comprising a nucleotide sequence, wherein said set of nucleic acid molecules together encode an antibody of any one of claims 1 to 30, or (iii) one or more recombinant expression vectors comprising one or more of said nucleic acid molecules, under conditions suitable for the expression of the encoded antibody; and
(ii) isolating or obtaining the antibody from the host cell or from the growth medium/supernatant.

34. An antibody as defined in any one of claims 1 to 30 for use in therapy.

35. An antibody as defined in any one of claims 1 to 30 for use in pain therapy.

36. A method of treating pain, said method comprising administering to a patient in need thereof a therapeutically effective amount of an antibody as defined in any one of claims 1 to 30.

37. Use of an antibody as defined in any one of claims 1 to 30 in the manufacture of a medicament for use in therapy.

38. The use according to claim 37, wherein said therapy is the treatment of pain.

39. An isolated peptide, wherein said isolated peptide is as defined in any one of claims 2 to 7.

40. A conjugate comprising an isolated peptide as defined in any one of claims 2 to 7 and a peptide carrier.

41. The conjugate of claim 40, wherein said peptide carrier is keyhole limpet hemocyanin (KLH), ovalbumin (OVA) or bovine serum albumin (BSA).
